SEQUENCE LISTING (508 sequences)

SEQ ID NO: 1 - DNA, 20 bp, Artificial Sequence
  primer
gacsgatgggcccttggtgg

SEQ ID NO: 2 - DNA, 20 bp, Artificial Sequence
  Primer
gctgtaggtgctgtctttgc

SEQ ID NO: 3 - DNA, 39 bp, Artificial Sequence
  Primer
tattcccatggcgcgccsaggtccarctgcarcagyctg

SEQ ID NO: 4 - DNA, 37 bp, Artificial Sequence
  Primer
tattcccatggcgcgccgargtgmagctkgtkgagtc

SEQ ID NO: 5 - DNA, 37 bp, Artificial Sequence
  Primer
tattcccatggcgcgccsaggtgcagctkmaggagtc

SEQ ID NO: 6 - DNA, 37 bp, Artificial Sequence
  Primer
tattcccatggcgcgcccaggttactctgaaagagtc

SEQ ID NO: 7 - DNA, 39 bp, Artificial Sequence
  Primer
tattcccatggcgcgcccagatccagttggtgcagtctg

SEQ ID NO: 8 - DNA, 45 bp, Artificial Sequence
  Primer
ggcgcgccatgggaatagctagccgayatccagatgachcarwct

SEQ ID NO: 9 - DNA, 44 bp, Artificial Sequence
  Primer
ggcgcgccatgggaatagctagccracattgtgmtgachcagtc

SEQ ID NO: 10 - DNA, 46 bp, Artificial Sequence
  Primer
ggcgcgccatgggaatagctagccsamattgtkctsacccartctc

SEQ ID NO: 11 - DNA, 45 bp, Artificial Sequence
  Primer
ggcgcgccatgggaatagctagccgatrttgtgatgacbcarrct

SEQ ID NO: 12 - DNA, 24 bp, Artificial Sequence
  Primer
ggacagggmtccakagttccadkt

SEQ ID NO: 13 - DNA, 43 bp, Artificial Sequence
  Primer
gacagatggtgcagccacagttcgtttgatttccagcttggtg

SEQ ID NO: 14 - DNA, 43 bp, Artificial Sequence
  Primer
gacagatggtgcagccacagttcgttttatttccagcttggtc

SEQ ID NO: 15 - DNA, 43 bp, Artificial Sequence
  Primer
gacagatggtgcagccacagttcgttttatttccaactttgtc

SEQ ID NO: 16 - DNA, 43 bp, Artificial Sequence
  Primer
gacagatggtgcagccacagttcgtttcagctccagcttggtc

SEQ ID NO: 17 - DNA, 23 bp, Artificial Sequence
  Primer
gaactgtggctgcaccatctgtc

SEQ ID NO: 18 - DNA, 43 bp, Artificial Sequence
  Primer
accgcctccaccggcggccgcttattaacactctcccctgttg

SEQ ID NO: 19 - DNA, 51 bp, Artificial Sequence
  Primer
accgcctccaccggcggccgcttattaacactctcccctgttgaagctctt

SEQ ID NO: 20 - DNA, 30 bp, Artificial Sequence
  Primer
ggaggcgctcgagacggtgaccgtggtccc

SEQ ID NO: 21 - DNA, 30 bp, Artificial Sequence
  Primer
ggaggcgctcgagactgtgagagtggtgcc

SEQ ID NO: 22 - DNA, 30 bp, Artificial Sequence
  Primer
ggaggcgctcgagacagtgaccagagtccc

SEQ ID NO: 23 - DNA, 30 bp, Artificial Sequence
  Primer
ggaggcgctcgagacggtgactgaggttcc

SEQ ID NO: 24 - protein, 10 aa, Mus musculus
SGYSFTGYTM

SEQ ID NO: 25 - protein, 10 aa, Mus musculus
SGYSFTDYTM

SEQ ID NO: 26 - protein, 10 aa, Mus musculus
SGFTFSNYAM

SEQ ID NO: 27 - protein, 10 aa, Mus musculus
SGFTFSSYAM

SEQ ID NO: 28 - protein, 10 aa, Mus musculus
SGYSFTAYNI

SEQ ID NO: 29 - protein, 10 aa, Mus musculus
SGYSFTAYSM

SEQ ID NO: 30 - protein, 10 aa, Mus musculus
SGYTFTNFAI

SEQ ID NO: 31 - protein, 10 aa, Mus musculus
SGFNIKDTYM

SEQ ID NO: 32 - protein, 10 aa, Mus musculus
SGYSFTSYWM

SEQ ID NO: 33 - protein, 10 aa, Mus musculus
SGFSLTNYDV

SEQ ID NO: 34 - protein, 10 aa, Mus musculus
SGFTFSNYGM

SEQ ID NO: 35 - protein, 10 aa, Mus musculus
SGYIFANYGM

SEQ ID NO: 36 - protein, 10 aa, Mus musculus
SGYNFTNYGM

SEQ ID NO: 37 - protein, 10 aa, Mus musculus
SGYTFTNYGM

SEQ ID NO: 38 - protein, 10 aa, Mus musculus
SGYTFTDYYI

SEQ ID NO: 39 - protein, 10 aa, Mus musculus
SGNTFTNFYL

SEQ ID NO: 40 - protein, 10 aa, Mus musculus
SEFTFSNYAM

SEQ ID NO: 41 - protein, 10 aa, Mus musculus
SGYTFTSYRM

SEQ ID NO: 42 - protein, 10 aa, Mus musculus
SGYMFTNHGM

SEQ ID NO: 43 - protein, 10 aa, Mus musculus
SGYMFTNYGM

SEQ ID NO: 44 - protein, 10 aa, Mus musculus
SGYIFTNYGM

SEQ ID NO: 45 - protein, 10 aa, Mus musculus
SGFNIKDYYI

SEQ ID NO: 46 - protein, 10 aa, Mus musculus
SGYTFINYGM

SEQ ID NO: 47 - protein, 10 aa, Mus musculus
SGYTFTDYFI

SEQ ID NO: 48 - protein, 10 aa, Mus musculus
SGYIFTGYNI

SEQ ID NO: 49 - protein, 10 aa, Mus musculus
LINPYNGGTT

SEQ ID NO: 50 - protein, 10 aa, Mus musculus
LINPYNGGTM

SEQ ID NO: 51 - protein, 9 aa, Mus musculus
SISSGGNTF

SEQ ID NO: 52 - protein, 9 aa, Mus musculus
SISSGGSTY

SEQ ID NO: 53 - protein, 10 aa, Mus musculus
SIDPYYGDTK

SEQ ID NO: 54 - protein, 10 aa, Mus musculus
LISSNSGDVS

SEQ ID NO: 55 - protein, 10 aa, Mus musculus
LISTSSGDVS

SEQ ID NO: 56 - protein, 10 aa, Mus musculus
RIDPANGNTK

SEQ ID NO: 57 - protein, 10 aa, Mus musculus
MIHPSDSETR

SEQ ID NO: 58 - protein, 9 aa, Mus musculus
VIWSGGNTD

SEQ ID NO: 59 - protein, 10 aa, Mus musculus
AINSNGDITY

SEQ ID NO: 60 - protein, 10 aa, Mus musculus
LINPYNGGTR

SEQ ID NO: 61 - protein, 10 aa, Mus musculus
WINTYTGEPT

SEQ ID NO: 62 - protein, 10 aa, Mus musculus
WIYPGGGNTR

SEQ ID NO: 63 - protein, 10 aa, Mus musculus
CIYPGNVKTK

SEQ ID NO: 64 - protein, 10 aa, Mus musculus
TISSGGSYTY

SEQ ID NO: 65 - protein, 10 aa, Mus musculus
RIDPYDSGTH

SEQ ID NO: 66 - protein, 10 aa, Mus musculus
WIDPENGRTE

SEQ ID NO: 67 - protein, 10 aa, Mus musculus
EIYPGSSNTY

SEQ ID NO: 68 - protein, 10 aa, Mus musculus
AVYPGNGDTS

SEQ ID NO: 69 - protein, 15 aa, Mus musculus
CARDYYGSSPDFDYW

SEQ ID NO: 70 - protein, 15 aa, Mus musculus
CARDNYGSSPDFDYW

SEQ ID NO: 71 - protein, 15 aa, Mus musculus
CARDNYGSSPYFDYW

SEQ ID NO: 72 - protein, 16 aa, Mus musculus
CVRYYYGVTYWYFDVW

SEQ ID NO: 73 - protein, 16 aa, Mus musculus
CVRYYYGIRYWYFDVW

SEQ ID NO: 74 - protein, 16 aa, Mus musculus
CARRMITMGDWYFDVW

SEQ ID NO: 75 - protein, 16 aa, Mus musculus
CARRMITTGDWYFDVW

SEQ ID NO: 76 - protein, 14 aa, Mus musculus
CARHYGAHNYFDYW

SEQ ID NO: 77 - protein, 14 aa, Mus musculus
CARHYGANNYFDYW

SEQ ID NO: 78 - protein, 16 aa, Mus musculus
CAREENYYGTYYFDYW

SEQ ID NO: 79 - protein, 14 aa, Mus musculus
CARWGDHDDAMDFW

SEQ ID NO: 80 - protein, 17 aa, Mus musculus
CARNHGDGYFNWYFDVW

SEQ ID NO: 81 - protein, 17 aa, Mus musculus
CARNHGDGYYNWYFDVW

SEQ ID NO: 82 - protein, 11 aa, Mus musculus
CARGTAWFTYW

SEQ ID NO: 83 - protein, 14 aa, Mus musculus
CARDGDDGWDIDVW

SEQ ID NO: 84 - protein, 13 aa, Mus musculus
CARRGTYWHFDVW

SEQ ID NO: 85 - protein, 13 aa, Mus musculus
CARRGSYWHFDVW

SEQ ID NO: 86 - protein, 12 aa, Mus musculus
CARRSTLVFDYW

SEQ ID NO: 87 - protein, 12 aa, Mus musculus
CARNGYWYFDVW

SEQ ID NO: 88 - protein, 16 aa, Mus musculus
CAKEGDYDGTAYFDYW

SEQ ID NO: 89 - protein, 12 aa, Mus musculus
CARRRDGNFDYW

SEQ ID NO: 90 - protein, 10 aa, Mus musculus
CVRHGYFDVW

SEQ ID NO: 91 - protein, 9 aa, Mus musculus
CAFYDGAYW

SEQ ID NO: 92 - protein, 9 aa, Mus musculus
CASYDPDYW

SEQ ID NO: 93 - protein, 14 aa, Mus musculus
CARDTTATYYFDYW

SEQ ID NO: 94 - protein, 12 aa, Mus musculus
CARRVATYFDVW

SEQ ID NO: 95 - protein, 12 aa, Mus musculus
CTRRSHITLDYW

SEQ ID NO: 96 - protein, 12 aa, Mus musculus
CARRRTTAFDYW

SEQ ID NO: 97 - protein, 15 aa, Mus musculus
CNNGNYVRHYYFDYW

SEQ ID NO: 98 - protein, 12 aa, Mus musculus
CTRRREITFDYW

SEQ ID NO: 99 - protein, 12 aa, Mus musculus
CARSGISPFTYW

SEQ ID NO: 100 - protein, 11 aa, Mus musculus
CAKYDRGFASW

SEQ ID NO: 101 - protein, 8 aa, Mus musculus
SQGISNHL

SEQ ID NO: 102 - protein, 8 aa, Mus musculus
SQGIRNYL

SEQ ID NO: 103 - protein, 8 aa, Mus musculus
SQGINNYL

SEQ ID NO: 104 - protein, 12 aa, Mus musculus
SQSVDHDGDSYM

SEQ ID NO: 105 - protein, 12 aa, Mus musculus
SQSVDYDGDSYM

SEQ ID NO: 106 - protein, 8 aa, Mus musculus
SQDISNYL

SEQ ID NO: 107 - protein, 8 aa, Mus musculus
SQDISTYL

SEQ ID NO: 108 - protein, 9 aa, Mus musculus
TSSISSSYL

SEQ ID NO: 109 - protein, 9 aa, Mus musculus
NSSVSSSYL

SEQ ID NO: 110 - protein, 8 aa, Mus musculus
SENIYGYF

SEQ ID NO: 111 - protein, 8 aa, Mus musculus
SQDINNYI

SEQ ID NO: 112 - protein, 8 aa, Mus musculus
SQDINKYI

SEQ ID NO: 113 - protein, 8 aa, Mus musculus
SENIYSYL

SEQ ID NO: 114 - protein, 8 aa, Mus musculus
SQDVRTDV

SEQ ID NO: 115 - protein, 8 aa, Mus musculus
SQDVITAV

SEQ ID NO: 116 - protein, 8 aa, Mus musculus
SQSIGTSI

SEQ ID NO: 117 - protein, 12 aa, Mus musculus
SSQSLLNQKNYL

SEQ ID NO: 118 - protein, 9 aa, Mus musculus
SSSVSSSYL

SEQ ID NO: 119 - protein, 14 aa, Mus musculus
SQSLLYSSDQKNYL

SEQ ID NO: 120 - protein, 7 aa, Mus musculus
NSSVSYM

SEQ ID NO: 121 - protein, 7 aa, Mus musculus
SSSLSYM

SEQ ID NO: 122 - protein, 8 aa, Mus musculus
SQRIGTSM

SEQ ID NO: 123 - protein, 8 aa, Mus musculus
SQNIGTSI

SEQ ID NO: 124 - protein, 7 aa, Mus musculus
ISSVSYM

SEQ ID NO: 125 - protein, 8 aa, Mus musculus
SQTIATSI

SEQ ID NO: 126 - protein, 13 aa, Mus musculus
SQSLVHSNGNTYL

SEQ ID NO: 127 - protein, 12 aa, Mus musculus
NESVEYSGTSLM

SEQ ID NO: 128 - protein, 5 aa, Mus musculus
YFTSS

SEQ ID NO: 129 - protein, 5 aa, Mus musculus
YYTSS

SEQ ID NO: 130 - protein, 5 aa, Mus musculus
YAASN

SEQ ID NO: 131 - protein, 5 aa, Mus musculus
YYTSR

SEQ ID NO: 132 - protein, 5 aa, Mus musculus
FYTSR

SEQ ID NO: 133 - protein, 5 aa, Mus musculus
YGTSN

SEQ ID NO: 134 - protein, 5 aa, Mus musculus
YGTSN

SEQ ID NO: 135 - protein, 5 aa, Mus musculus
YNANS

SEQ ID NO: 136 - protein, 5 aa, Mus musculus
YNAKT

SEQ ID NO: 137 - protein, 5 aa, Mus musculus
HYTST

SEQ ID NO: 138 - protein, 5 aa, Mus musculus
YHTST

SEQ ID NO: 139 - protein, 5 aa, Mus musculus
YSASF

SEQ ID NO: 140 - protein, 5 aa, Mus musculus
YSASY

SEQ ID NO: 141 - protein, 5 aa, Mus musculus
KSASE

SEQ ID NO: 142 - protein, 5 aa, Mus musculus
YWAST

SEQ ID NO: 143 - protein, 5 aa, Mus musculus
YSTSN

SEQ ID NO: 144 - protein, 5 aa, Mus musculus
KNASE

SEQ ID NO: 145 - protein, 5 aa, Mus musculus
YDTSK

SEQ ID NO: 146 - protein, 5 aa, Mus musculus
YDTSN

SEQ ID NO: 147 - protein, 5 aa, Mus musculus
KDASE

SEQ ID NO: 148 - protein, 5 aa, Mus musculus
YATSN

SEQ ID NO: 149 - protein, 5 aa, Mus musculus
YKVSN

SEQ ID NO: 150 - protein, 5 aa, Mus musculus
SAASN

SEQ ID NO: 151 - protein, 11 aa, Mus musculus
CQQYSNLPYTF

SEQ ID NO: 152 - protein, 11 aa, Mus musculus
CQQYSKIPYTC

SEQ ID NO: 153 - protein, 10 aa, Mus musculus
CQQNYEDPTF

SEQ ID NO: 154 - protein, 10 aa, Mus musculus
CQQSNEDPTF

SEQ ID NO: 155 - protein, 11 aa, Mus musculus
CQQGDALPWTF

SEQ ID NO: 156 - protein, 11 aa, Mus musculus [sequence omitted]

SEQ ID NO: 157 - protein, 11 aa, Mus musculus
CQQWSSRPPTF

SEQ ID NO: 158 - protein, 11 aa, Mus musculus [sequence omitted]

SEQ ID NO: 159 - protein, 11 aa, Mus musculus [sequence omitted]

SEQ ID NO: 160 - protein, 11 aa, Mus musculus
CQQRSYFPFTF

SEQ ID NO: 161 - protein, 11 aa, Mus musculus
CKQVYDVPFTF

SEQ ID NO: 162 - protein, 11 aa, Mus musculus
CQHHYGTPFTF

SEQ ID NO: 163 - protein, 10 aa, Mus musculus
CLQYDNLWTF

SEQ ID NO: 164 - protein, 11 aa, Mus musculus
CQHHYGYPYTF

SEQ ID NO: 165 - protein, 11 aa, Mus musculus [sequence omitted]

SEQ ID NO: 166 - protein, 11 aa, Mus musculus
CQQHYTSPWTF

SEQ ID NO: 167 - protein, 11 aa, Mus musculus [sequence omitted]

SEQ ID NO: 168 - protein, 11 aa, Mus musculus
CQQSNRWPLTF

SEQ ID NO: 169 - protein, 11 aa, Mus musculus
CQNDYDYPYTF

SEQ ID NO: 170 - protein, 11 aa, Mus musculus
CHQYHRSPLTF

SEQ ID NO: 171 - protein, 11 aa, Mus musculus
CQQTFDVPWTF

SEQ ID NO: 172 - protein, 11 aa, Mus musculus
CQQSNSWPLTY

SEQ ID NO: 173 - protein, 11 aa, Mus musculus [sequence omitted]

SEQ ID NO: 174 - protein, 11 aa, Mus musculus
CQQWSSNPFTF

SEQ ID NO: 175 - protein, 11 aa, Mus musculus
CKQAYDVPWTF

SEQ ID NO: 176 - protein, 11 aa, Mus musculus
CQQWSSFPPTF

SEQ ID NO: 177 - protein, 11 aa, Mus musculus
CQQSNSWPLTF

SEQ ID NO: 178 - protein, 11 aa, Mus musculus
CQQSDSWPLTF

SEQ ID NO: 179 - protein, 11 aa, Mus musculus
CQQWSSNPRTF

SEQ ID NO: 180 - protein, 11 aa, Mus musculus
CWQNTHFPQTF

SEQ ID NO: 181 - protein, 11 aa, Mus musculus
CQQSRQVPLTF

SEQ ID NO: 182 - DNA, 30 bp, Mus musculus
tctggttactcattcactggctacaccatg

SEQ ID NO: 183 - DNA, 30 bp, Mus musculus
tcaggttactcattcactgactacaccatg

SEQ ID NO: 184 - DNA, 30 bp, Mus musculus
tcaggttattcattcaccggctacaccatg

SEQ ID NO: 185 - DNA, 30 bp, Mus musculus
tctggattcactttcagtaactatgccatg

SEQ ID NO: 186 - DNA, 30 bp, Mus musculus
tctggattcactttcagtagctatgccatg

SEQ ID NO: 187 - DNA, 30 bp, Mus musculus
tctggttattcattcactgcctacaacatt

SEQ ID NO: 188 - DNA, 30 bp, Mus musculus
tctggttactcattcactgcctacagcatg

SEQ ID NO: 189 - DNA, 30 bp, Mus musculus
tctggctacacattcactaattttgctatt

SEQ ID NO: 190 - DNA, 30 bp, Mus musculus
tctggcttcaacattaaagacacctatatg

SEQ ID NO: 191 - DNA, 30 bp, Mus musculus
tctggctactccttcaccagttactggatg

SEQ ID NO: 192 - DNA, 30 bp, Mus musculus
tctggtttctcattaactaactatgatgta

SEQ ID NO: 193 - DNA, 30 bp, Mus musculus
tctggattcactttcagtaactatggcatg

SEQ ID NO: 194 - DNA, 30 bp, Mus musculus
tctgggtatattttcgcaaactatggcatg

SEQ ID NO: 195 - DNA, 30 bp, Mus musculus
tctgggtataacttcacaaactatggaatg

SEQ ID NO: 196 - DNA, 30 bp, Mus musculus
tctggttataccttcacaaactatggaatg

SEQ ID NO: 197 - DNA, 30 bp, Mus musculus
tctggctacaccttcactgactactatata

SEQ ID NO: 198 - DNA, 30 bp, Mus musculus
tctggcaacaccttcacaaacttctattta

SEQ ID NO: 199 - DNA, 30 bp, Mus musculus
tctgggtataccttcacaaactatggaatg

SEQ ID NO: 200 - DNA, 30 bp, Mus musculus
tctgaattcactttcagtaactatgccatg

SEQ ID NO: 201 - DNA, 30 bp, Mus musculus
tctggctacacgttcaccagctacaggatg

SEQ ID NO: 202 - DNA, 30 bp, Mus musculus
tctggttactcattcactgactacaccatg

SEQ ID NO: 203 - DNA, 30 bp, Mus musculus
tctgggtatatgttcacaaaccatggaatg

SEQ ID NO: 204 - DNA, 30 bp, Mus musculus
tctgggtatatgttcacaaactatggaatg

SEQ ID NO: 205 - DNA, 30 bp, Mus musculus
tctgggtatatcttcacaaactatggaatg

SEQ ID NO: 206 - DNA, 30 bp, Mus musculus
tctggcttcaacattaaagactactatata

SEQ ID NO: 207 - DNA, 30 bp, Mus musculus
tctgggtataccttcataaattatggaatg

SEQ ID NO: 208 - DNA, 30 bp, Mus musculus
tctggctacaccttcactgactactttata

SEQ ID NO: 209 - DNA, 30 bp, Mus musculus
tctggctacatttttaccggttacaatata

SEQ ID NO: 210 - DNA, 30 bp, Mus musculus
cttattaatccttacaatggtggtactacc

SEQ ID NO: 211 - DNA, 30 bp, Mus musculus
cttattaatccttacaatggtggtactatg

SEQ ID NO: 212 - DNA, 27 bp, Mus musculus
tccattagtagtggtggtaacaccttt

SEQ ID NO: 213 - DNA, 24 bp, Mus musculus
tccattagtagtggtggtagcacc

SEQ ID NO: 214 - DNA, 30 bp, Mus musculus
agtattgatccttactatggtgatactaaa

SEQ ID NO: 215 - DNA, 30 bp, Mus musculus
agtattgatccttattatggtgatactaag

SEQ ID NO: 216 - DNA, 30 bp, Mus musculus
cttattagttctaactctggtgatgttagc

SEQ ID NO: 217 - DNA, 30 bp, Mus musculus
cttattagtacttcctctggtgatgttagc

SEQ ID NO: 218 - DNA, 30 bp, Mus musculus
aggattgatcctgcgaatggtaatactaaa

SEQ ID NO: 219 - DNA, 30 bp, Mus musculus
atgattcatccttccgatagtgaaactagg

SEQ ID NO: 220 - DNA, 27 bp, Mus musculus
gtgatttggagtggtggaaacacagac

SEQ ID NO: 221 - DNA, 27 bp, Mus musculus
gtgatatggaattatggaaacacagac

SEQ ID NO: 222 - DNA, 27 bp, Mus musculus
gccattaatagtaatggtgatattacc

SEQ ID NO: 223 - DNA, 30 bp, Mus musculus
cttattaatccttacaatggtggtactaga

SEQ ID NO: 224 - DNA, 30 bp, Mus musculus
tggataaacacctacactggagagccaaca

SEQ ID NO: 225 - DNA, 30 bp, Mus musculus
tggattaacacctacactggagagccaaca

SEQ ID NO: 226 - DNA, 30 bp, Mus musculus
tggataaacacttacactggagagccaaca

SEQ ID NO: 227 - DNA, 30 bp, Mus musculus
tggatttatcctggaggcggtaatactagg

SEQ ID NO: 228 - DNA, 30 bp, Mus musculus
tgtatttatcctggaaacgttaagactaaa

SEQ ID NO: 229 - DNA, 30 bp, Mus musculus
tggataaacacctacactggagagcctaca

SEQ ID NO: 230 - DNA, 27 bp, Mus musculus
accattagtagtggtggtagttacacc

SEQ ID NO: 231 - DNA, 30 bp, Mus musculus
aggattgatccttacgatagtggaactcac

SEQ ID NO: 232 - DNA, 30 bp, Mus musculus
cttattaatccttacaatggtggtactagg

SEQ ID NO: 233 - DNA, 30 bp, Mus musculus
tggattgatcctgagaatggtcgtactgaa

SEQ ID NO: 234 - DNA, 30 bp, Mus musculus
gagatttatcctggaagtagtaatacttac

SEQ ID NO: 235 - DNA, 30 bp, Mus musculus
gctgtttatccaggaaatggtgatacttcc

SEQ ID NO: 236 - DNA, 45 bp, Mus musculus
tgtgcaagagattactacggtagtagtccagactttgactactgg

SEQ ID NO: 237 - DNA, 45 bp, Mus musculus
tgtgcaagagataactacggtagtagtccagactttgactactgg

SEQ ID NO: 238 - DNA, 45 bp, Mus musculus
tgtgcaagagataactacggtagtagcccatactttgactactgg

SEQ ID NO: 239 - DNA, 48 bp, Mus musculus
tgtgtccgttattactacggtgttacctactggtacttcgatgtctgg

SEQ ID NO: 240 - DNA, 48 bp, Mus musculus
tgtgtccgttattactacggtattaggtactggtacttcgatgtctgg

SEQ ID NO: 241 - DNA, 48 bp, Mus musculus
tgtgcaagaaggatgattacgatgggagactggtatttcgatgtctgg

SEQ ID NO: 242 - DNA, 48 bp, Mus musculus
tgtgcaagaaggatgattacgacgggagactggtacttcgatgtctgg

SEQ ID NO: 243 - DNA, 42 bp, Mus musculus
tgtgcaagacactatggtgcccacaactattttgactattgg

SEQ ID NO: 244 - DNA, 42 bp, Mus musculus
tgtgcaagacactatggtgccaacaactattttgactattgg

SEQ ID NO: 245 - DNA, 48 bp, Mus musculus
tgtgctagagaggagaattactacggtacctactactttgactactgg

SEQ ID NO: 246 - DNA, 42 bp, Mus musculus
tgtgcaagatggggggatcacgacgatgctatggacttctgg

SEQ ID NO: 247 - DNA, 51 bp, Mus musculus
tgtgccagaaatcatggtgatggttacttcaactggtacttcgatgtctgg

SEQ ID NO: 248 - DNA, 51 bp, Mus musculus
tgtgccagaaatcatggtgatggttactataactggtacttcgatgtctgg

SEQ ID NO: 249 - DNA, 33 bp, Mus musculus
tgtgcaagaggaactgcctggtttacttactgg

SEQ ID NO: 250 - DNA, 42 bp, Mus musculus
tgtgcaagagatggggatgatggttgggacatcgatgtctgg

SEQ ID NO: 251 - DNA, 39 bp, Mus musculus
tgtgcaagaagggggacttactggcacttcgatgtctgg

SEQ ID NO: 252 - DNA, 39 bp, Mus musculus
tgtgcaagaagggggtcttactggcacttcgatgtctgg

SEQ ID NO: 253 - DNA, 36 bp, Mus musculus
tgtgcaagacgctctacgctcgtctttgactactgg

SEQ ID NO: 254 - DNA, 36 bp, Mus musculus
tgtgcaagaaacggctactggtacttcgatgtctgg

SEQ ID NO: 255 - DNA, 48 bp, Mus musculus
tgtgcaaaggagggagattacgacgggacggcctactttgattactgg

SEQ ID NO: 256 - DNA, 36 bp, Mus musculus
tgtgcaagaaggcgggacggaaactttgactactgg

SEQ ID NO: 257 - DNA, 30 bp, Mus musculus
tgtgtaagacatggatacttcgatgtctgg

SEQ ID NO: 258 - DNA, 27 bp, Mus musculus
tgtgccttctatgatggggcttactgg

SEQ ID NO: 259 - DNA, 27 bp, Mus musculus
tgtgctagttatgatcctgactactgg

SEQ ID NO: 260 - DNA, 42 bp, Mus musculus
tgtgcaagagatactacggcgacgtactactttgactactgg

SEQ ID NO: 261 - DNA, 36 bp, Mus musculus
tgtgcaagacgtgttgcgacgtacttcgatgtctgg

SEQ ID NO: 262 - DNA, 36 bp, Mus musculus
tgtacacgaaggagtcatattaccttggactactgg

SEQ ID NO: 263 - DNA, 45 bp, Mus musculus
tgtaataatggtaactacgtcagacactactactttgactactgg

SEQ ID NO: 264 - DNA, 36 bp, Mus musculus
tgtacaagaagaagagaaataacctttgactactgg

SEQ ID NO: 265 - DNA, 36 bp, Mus musculus
tgtgcaagatcggggatttcgccctttacttactgg

SEQ ID NO: 266 - DNA, 33 bp, Mus musculus
tgtgcaaaatatgaccgggggtttgcttcctgg

SEQ ID NO: 267 - DNA, 24 bp, Mus musculus
agtcagggcattagcaatcattta

SEQ ID NO: 268 - DNA, 24 bp, Mus musculus
agtcagggcattagaaattattta

SEQ ID NO: 269 - DNA, 24 bp, Mus musculus
agtcagggcattaacaattattta

SEQ ID NO: 270 - DNA, 36 bp, Mus musculus
agccaaagtgttgatcatgatggtgatagttatatg

SEQ ID NO: 271 - DNA, 36 bp, Mus musculus
agccaaagtgttgattatgatggtgatagttatatg

SEQ ID NO: 272 - DNA, 24 bp, Mus musculus
agtcaggacattagcaattattta

SEQ ID NO: 273 - DNA, 24 bp, Mus musculus
agtcaggacattagtacttattta

SEQ ID NO: 274 - DNA, 27 bp, Mus musculus
acctcaagtataagttccagttacttg

SEQ ID NO: 275 - DNA, 27 bp, Mus musculus
aactcaagtgtaagttccagttacttg

SEQ ID NO: 276 - DNA, 24 bp, Mus musculus
agtgagaacatttactacaattta

SEQ ID NO: 277 - DNA, 24 bp, Mus musculus
agtgaaaatatttacggttatttc

SEQ ID NO: 278 - DNA, 24 bp, Mus musculus
agtcaagacattaacaactatata

SEQ ID NO: 279 - DNA, 24 bp, Mus musculus
agtcaagacattaacaagtatata

SEQ ID NO: 280 - DNA, 24 bp, Mus musculus
agtgagaatatttacagttattta

SEQ ID NO: 281 - DNA, 24 bp, Mus musculus
agtcaggatgtgaggactgatgta

SEQ ID NO: 282 - DNA, 24 bp, Mus musculus
agtcaggatgtgattactgctgta

SEQ ID NO: 283 - DNA, 24 bp, Mus musculus
agtcagagcattggtacaagcata

SEQ ID NO: 284 - DNA, 36 bp, Mus musculus
tccagtcagagtctcttaaatcaaaagaactacttg

SEQ ID NO: 285 - DNA, 27 bp, Mus musculus
agctcaagtgtaagttccagttacttg

SEQ ID NO: 286 - DNA, 24 bp, Mus musculus
agtgagaacatttactacaattta

SEQ ID NO: 287 - DNA, 42 bp, Mus musculus
agtcagagccttttatatagtagcgatcaaaagaactacttg

SEQ ID NO: 288 - DNA, 21 bp, Mus musculus
aactcaagtgtaagttacatg

SEQ ID NO: 289 - DNA, 21 bp, Mus musculus
agctcaagtttaagttacatg

SEQ ID NO: 290 - DNA, 24 bp, Mus musculus
agtcagcgcattggcacaagcatg

SEQ ID NO: 291 - DNA, 24 bp, Mus musculus
agtcagagcattggcacaagcata

SEQ ID NO: 292 - DNA, 24 bp, Mus musculus
agtcagaacattggcacaagcata

SEQ ID NO: 293 - DNA, 21 bp, Mus musculus
atctcaagtgtaagttacatg

SEQ ID NO: 294 - DNA, 24 bp, Mus musculus
agtcagaccattgccacaagcata

SEQ ID NO: 295 - DNA, 39 bp, Mus musculus
agtcagagccttgtacacagtaatggaaacacctattta

SEQ ID NO: 296 - DNA, 36 bp, Mus musculus
aatgaaagtgttgaatattctggcacaagtttaatg

SEQ ID NO: 297 - DNA, 15 bp, Mus musculus
tatttcacatcaagt

SEQ ID NO: 298 - DNA, 15 bp, Mus musculus
tattacacatcaagt

SEQ ID NO: 299 - DNA, 15 bp, Mus musculus
tatgctgcatccaat

SEQ ID NO: 300 - DNA, 15 bp, Mus musculus
tactacacatcaaga

SEQ ID NO: 301 - DNA, 15 bp, Mus musculus
ttctacacatcacga

SEQ ID NO: 302 - DNA, 15 bp, Mus musculus
tatggcacatccaac

SEQ ID NO: 303 - DNA, 15 bp, Mus musculus
tataatgcaaacagc

SEQ ID NO: 304 - DNA, 15 bp, Mus musculus
tataatgcaaaaacc

SEQ ID NO: 305 - DNA, 15 bp, Mus musculus
cattacacatctaca

SEQ ID NO: 306 - DNA, 15 bp, Mus musculus
tatcacacatcaact

SEQ ID NO: 307 - DNA, 15 bp, Mus musculus
tactcggcatccttc

SEQ ID NO: 308 - DNA, 15 bp, Mus musculus
tactcggcatcctac

SEQ ID NO: 309 - DNA, 15 bp, Mus musculus
aagagtgcttctgag

SEQ ID NO: 310 - DNA, 14 bp, Mus musculus
actgggcatccact

SEQ ID NO: 311 - DNA, 15 bp, Mus musculus
tatagcacatccaac

SEQ ID NO: 312 - DNA, 15 bp, Mus musculus
tataatgcaaacagt

SEQ ID NO: 313 - DNA, 15 bp, Mus musculus
aagaatgcttctgag

SEQ ID NO: 314 - DNA, 15 bp, Mus musculus
tatgacacatccaaa

SEQ ID NO: 315 - DNA, 15 bp, Mus musculus
tatgacacatccaac

SEQ ID NO: 316 - DNA, 15 bp, Mus musculus
aagtctgcttctgag

SEQ ID NO: 317 - DNA, 15 bp, Mus musculus
aaggatgcttctgag

SEQ ID NO: 318 - DNA, 15 bp, Mus musculus
tatgccacttccaac

SEQ ID NO: 319 - DNA, 15 bp, Mus musculus
tacaaagtttccaat

SEQ ID NO: 320 - DNA, 15 bp, Mus musculus
tctgctgcatccaac

SEQ ID NO: 321 - DNA, 33 bp, Mus musculus
tgtcagcagtatagtaaccttccgtacacgttc

SEQ ID NO: 322 - DNA, 33 bp, Mus musculus
tgtcagcagtatagtaagattccgtacacgtgc

SEQ ID NO: 323 - DNA, 30 bp, Mus musculus
tgtcagcaaaattatgaggatccgacgttc

SEQ ID NO: 324 - DNA, 30 bp, Mus musculus
tgtcagcaaagtaatgaggatccgacgttc

SEQ ID NO: 325 - DNA, 33 bp, Mus musculus
tgtcaacagggtgatgcgcttccgtggacgttc

SEQ ID NO: 326 - DNA, 33 bp, Mus musculus
tgccaacagggtaattcgcttccgttcacgttc

SEQ ID NO: 327 - DNA, 33 bp, Mus musculus
tgccagcagtggagtagtagaccacccacgttc

SEQ ID NO: 328 - DNA, 33 bp, Mus musculus
tgccagcagtacagtggttacccactcacgttc

SEQ ID NO: 329 - DNA, 33 bp, Mus musculus
tgccagcagtacagtgattacccactcacgttc

SEQ ID NO: 330 - DNA, 33 bp, Mus musculus
tgccagcaaaggagttatttcccgttcacgttc

SEQ ID NO: 331 - DNA, 33 bp, Mus musculus
tgtaaacaggtttatgacgttccattcacgttc

SEQ ID NO: 332 - DNA, 33 bp, Mus musculus
tgtcaacatcattatggtactccattcacgttc

SEQ ID NO: 333 - DNA, 30 bp, Mus musculus
tgtctacagtatgataatctgtggacgttc

SEQ ID NO: 334 - DNA, 30 bp, Mus musculus
tgtctacagtacgataatctgtggacgttc

SEQ ID NO: 335 - DNA, 33 bp, Mus musculus
tgtcaacatcattatggttatccgtatacgttc

SEQ ID NO: 336 - DNA, 33 bp, Mus musculus
tgtcagcagtatagtaaccttccgctcacgttc

SEQ ID NO: 337 - DNA, 33 bp, Mus musculus
tgtcagcaacattatacttctccgtggacgttc

SEQ ID NO: 338 - DNA, 33 bp, Mus musculus
tgtcagcaacattatagtactccgtggacgttc

SEQ ID NO: 339 - DNA, 33 bp, Mus musculus
tgtcaacaaagtaataggtggccgctcacgttc

SEQ ID NO: 340 - DNA, 33 bp, Mus musculus
tgtcagaatgattatgattatccttacacgttc

SEQ ID NO: 341 - DNA, 33 bp, Mus musculus
tgccaccagtatcatcgttccccgctcacgttc

SEQ ID NO: 342 - DNA, 33 bp, Mus musculus
tgtcaacagacttttgacgttccgtggacgttc

SEQ ID NO: 343 - DNA, 33 bp, Mus musculus
tgtcaacaaagtaatagctggccactcacgtac

SEQ ID NO: 344 - DNA, 33 bp, Mus musculus
tgtcagcaatattataactatccgctcacgttc

SEQ ID NO: 345 - DNA, 33 bp, Mus musculus
tgccagcagtggagtagtaacccattcacgttc

SEQ ID NO: 346 - DNA, 33 bp, Mus musculus
tgtaaacaggcttatgacgttccgtggacgttc

SEQ ID NO: 347 - DNA, 33 bp, Mus musculus
tgccagcagtggagtagtttcccaccgacattc

SEQ ID NO: 348 - DNA, 33 bp, Mus musculus
tgtcaacaaagtaatagttggccgctcacgttc

SEQ ID NO: 349 - DNA, 33 bp, Mus musculus
tgtcaacaaagtaatagctggccgctcacgttc

SEQ ID NO: 350 - DNA, 33 bp, Mus musculus
tgtcaacaaagtgatagctggccactcacgttc

SEQ ID NO: 351 - DNA, 33 bp, Mus musculus
tgccagcagtggagtagtaacccacggacgttc

SEQ ID NO: 352 - DNA, 33 bp, Mus musculus
tgtcaacaaagtaatagctggccactcacgttc

SEQ ID NO: 353 - DNA, 33 bp, Mus musculus
tgctggcaaaatacacattttcctcagacgttc

SEQ ID NO: 354 - DNA, 33 bp, Mus musculus
tgtcagcaaagtaggcaggttcctctcacgttc

SEQ ID NO: 355 - protein, 97 aa, Mus musculus
NIVLTQSTSSLSASLGDRVTISCSASQGIRNYLNWYQQKPDGTVKLLIYHTSTLHSGVPSRFSGSGSGTDYSLTISNLEPEDIATYYCQQYSNLPLT

SEQ ID NO: 356 - protein, 98 aa, Mus musculus
DIVLTQSPAIMSASPGEQVTMTCRATSSISSSYLHWYQQKSGASPKLWIYGTSNLASGVPTRFSGSGSGTSYSLTISSVEAEDAATYYCQQYSDYPLT

SEQ ID NO: 357 - protein, 97 aa, Mus musculus
DIVLTQSPASLSASVGESVTITCRPSENIYGYFAWYQQRQGKSPQLLVYNAKTLAEGVPSRFSGSGSGTHFSLKINSLQPEDFGTYYCQHHYGTPFT

SEQ ID NO: 358 - protein, 97 aa, Mus musculus
DIVLTQSPASLAASVGETVTITCRASENIYYNLAWYQQKQGKSPQLLIYNANSLEGGVPSRFSGSGSGTQYSMKINSMQPEDTATYFCKQAYDVPWT

SEQ ID NO: 359 - protein, 96 aa, Mus musculus
EIVLTQSPAIMSASPGEKVTMTCSASSSLSYMYWYQQKPGSSPRLLIYDTSNLASGVPFRFSGSGSGTSYSLTISRMEAEDAATYYCQQWSSFPPT

SEQ ID NO: 360 - protein, 98 aa, Mus musculus
EIVLTQSPAIMSASPGEQVTMTCRATSSISSSYLHWYQQKSGASPKLWIYGTSNLASGVPTRFSGGGSGTSYSLTISRMEAEDAATYYCQQRSYFPFT

SEQ ID NO: 361 - protein, 103 aa, Mus musculus
NIVMTQSPSSLAVSVGEKVTMSCKSSQSLLYSSDQKNYLAWYQLKPGQSPKLLIYWASTRESGVPDRFTGSGSGTDFTLTISSVKAEDLAVYYCQQYYNYPLT

SEQ ID NO: 362 - protein, 97 aa, Mus musculus
NIVLTQSPASLAASVGETVTITCRASENIYYNLAWYQQKQGKSPQLLIYNANSLEDGVPSRFSGSGSGTQYSMKINSMQPEDTATYFCKQVYDVPFT

SEQ ID NO: 363 - protein, 97 aa, Mus musculus
HIVLTQSPAILSVSPGERVSFSCRASQTIATSINWYQQRTNGSPRLLIKNASESISGIPSRFSGSGSGTDFTLTINSVESEDIADYYCQQSNSWPLT

SEQ ID NO: 364 - protein, 97 aa, Mus musculus
HIVLTQSPSSLSASLGDRVTISCRASQDISTYLNWYQQKPDGTVKLLIFYTSRLHAGVPSRFSGSGSGTHHSLTISNLEQEDIATYFCQQGNSLPFT

SEQ ID NO: 365 - protein, 96 aa, Mus musculus
DIVMTQSPSSLSESLGGKVTITCKASQDINNYIAWYQHKPGKGPRLLIHYTSTLLPGIPSRFSGSGSGTDYSFSISNLEPEDIATYYCLQYDNLWT

SEQ ID NO: 366 - protein, 96 aa, Mus musculus
DVVLTQSPAILSASPGEKVTMTCRAISSVSYMHWYQQKPGSSPKPWIYATSNLASGVPARFSGSGSGTSYSLTISRVEAEDAATYYCQQWSSNPRT

SEQ ID NO: 367 - protein, 97 aa, Mus musculus
NIVLTQSTSSLSASLGDRVTINCSASQGISNHLNWFQQKSDGTVKLLIYFTSSLHSGVPSRFSGSWSGTDYSLTISNLEPEDIAAYYCQQYSNLPYT

SEQ ID NO: 368 - protein, 97 aa, Mus musculus
NIVLTQSPAILSVSPGERVSFSCRASQNIGTSIHWYQQRTNGSPRFLVKDASESISGIPSRFSGSGSGTDFTLTINNVESEDIADYYCQQSDSWPLT

SEQ ID NO: 369 - protein, 97 aa, Mus musculus
NIVLTQSPASLAASVGETVTITCRVSENIYYNLAWYQQKQGKSPQLLIYNANSLEDGVPSRFSGSGSGTQYSMKINSMQPEDTATYFCQQTFDVPWT

SEQ ID NO: 370 - protein, 97 aa, Mus musculus
HIVLTQSPAILSVSPGERVSFSCRASQTIGTSIHWYQQRTNGSPRLLIKNASESISGIPSRFSGSGSGTDFTLSINSVESEDIADYYCQQSNSWPLT

SEQ ID NO: 371 - protein, 100 aa, Mus musculus
QIVLTQSPASLPASPGQRATISCKASQSVDYDGDSYMNWYHQKPGQPPKLLIYAASNLESGIPARFSGSGSGTDFTLNIHPVEEEDAATYYCQQSNEDPT

SEQ ID NO: 372 - protein, 97 aa, Mus musculus
NIVLTQSTSSLSASLGDRVTINCSASQGISNHLNWFQQKSDGTVKLLIYFTSSLHSGVPSRFSGSGSGTDYSLTISNLEPEDIAAYYCQQYSNLPYT

SEQ ID NO: 373 - protein, 100 aa, Mus musculus
NIVLTQSPASLAVSLGQRATISCKASQSVDHDGDSYMNWYQQKPGQSPKLLTYAASNLDSGIPARFSGSGSRTDFTLNIHPVEEEDAATYYCQQNYEDPT

SEQ ID NO: 374 - protein, 97 aa, Mus musculus
EIVLTQSPSSLSASLGDRVTISCRASQDISNYLNWYQRKPDGTVKLLIYYTSRLQSGVPSRFSGSGSGSEYSLTISNLDQEDIATYFCQQGDALPWT

SEQ ID NO: 375 - protein, 97 aa, Mus musculus
DIVLTQSPVILSVSPGERVSLSCRASQSIGTSINWYQQRTDGSPRLLIKSASESMSGIPSRFSGSGSGTDFTLSITSVESEDIADYYCQQSNSWPLT

SEQ ID NO: 376 - protein, 98 aa, Mus musculus
EIVLTQSPTIMSASPGEQVTMTCRTNSSVSSSYLHWYQQKSGASPKLWIYGTSNLASGVPTRFSGSGSGTSYSLTISSVEAGDAATYFCQQYSGYPLT

SEQ ID NO: 377 - protein, 97 aa, Mus musculus
NIVLTQSPAILSVSPGERVSFSCRASQRIGTSMNWYQQRTNGSPRLLIKSASESISGIPSRFSGSGSGTDFTLSINSVESDDVADYYCQQSNSWPLT

SEQ ID NO: 378 - protein, 98 aa, Mus musculus
DIVMTQSPAIMSASLGERVTMTCTASSSVSSSYLHWYQQKPGSSPKLWIYSTSNLASGVPARFSGSGSGTSYSLTISSMEAEDAATYYCHQYHRSPLT

SEQ ID NO: 379 - protein, 97 aa, Mus musculus
NIVLTQSTSSLSASLGDRVTISCSASQGIRNYLNWYQQKSDGTVKLLIYFTSSLHSGVPSRFSGSGSGTDYSLTISNLEPEDIAAYYCQQYSNLPYT

SEQ ID NO: 380 - protein, 101 aa, Mus musculus
NIVLTQSPASLAVSLGQRATISCRVNESVEYSGTSLMQWYQQKPGQPPKLLISAASNVESGVPARFSGRGSGTDFSLNIHPVEEDDIAMYFCQQSRQVPLT

SEQ ID NO: 381 - protein, 100 aa, Mus musculus
DIVLTQSPSSLTVTAGEKVTMSCKSSQSLLNQKNYLTWYQQKTGQPPKLLIYWASTRESGVPDRFTGSGSGTDFTLTISSVQAEDLAVYYCQNDYDYPYT

SEQ ID NO: 382 - protein, 98 aa, Mus musculus
NIVMTQSPAIMSASPGEQVTMTCRATSSISSSYLHWYQQKSGASPKLWIYGTSNLASGVPTRFSGSGSGTSYSLTISSMEAEDAATYYCQQWSSRPPT

SEQ ID NO: 383 - protein, 97 aa, Mus musculus
NIVMTQSPAILSVSPGERVSFSCRASQSIGTSINWYQQRTNASPRLLIKSASESISGIPSRFSGSGSGTDFTLNIKNVESEDIADYYCQQSNRWPLT

SEQ ID NO: 384 - protein, 102 aa, Mus musculus
MFVMTQTPLSLPVSLGDQASISCRSSQSLVHSNGNTYLHWYLQKPGQSPKLLIYKVSNRFSGVPDRFSGSGSGTDFTLKISRVEAEDLGVYYCWQNTHFPQT

SEQ ID NO: 385 - protein, 97 aa, Mus musculus
DIVLTQSPASLSASVGETVIITCRASENIYSYLVWYQQKQGKSPQLLVYNAKTLAEGVPSRFSGSGSGTQFSLKINSLQSEDFGSYSCQHHYGYPYT

SEQ ID NO: 386 - protein, 97 aa, Mus musculus
DIVLTQSTSSLSASLGDRVTINCSASQGISNHLNWFQQKSDGTVKLLIYFTSSLHSGVPSRFSGSGSGTDYSLTISNLEPEDIAAYYCQQYSNLPYT

SEQ ID NO: 387 - protein, 97 aa, Mus musculus
DIVMTQSTSSLSASLGDRVTISCSASQGINNYLNWYQQKPDGTVKLLIYYTSSLHSGVPPRFSGSGSGTDYSLTISNLEPEDIATYYCQQYSKIPYT

SEQ ID NO: 388 - protein, 97 aa, Mus musculus
HIVLTQSHKFMSTSVGDRVSITCKASQDVITAVTWSQQKPGQSPKLLIYSASYRYTGVPDRFTGSGSGTDFTFTISSVQAEDLAVYYCQQHYSTPWT

SEQ ID NO: 389 - protein, 96 aa, Mus musculus
DIVLTQSPAIMSASPGEKVTMTCSANSSVSYMLWYQQKSGTSPKRWIYDTSKLSSGVPARFSGSGSGTSYSLTISSMEAEDAATYYCQQWSSNPFT

SEQ ID NO: 390 - protein, 97 aa, Mus musculus
NIVMTQSHRFMSTSVGDRVSITCKASQDVRTDVAWFQQKPGQSPKLLIYSASFRYTGVPDRFTGSGSGTDFTLTISSVQAEDLAVYYCQQHYTSPWT

SEQ ID NO: 391 - protein, 96 aa, Mus musculus
NIVLTQSPSSLSESLGGKVTITCKASQDINKYIAWYQYKPGKGPRLLIHYTSTLQPGIPSRFSGSGSGRDYSFSISNLEPEDIATYYCLQYDNLWT

SEQ ID NO: 392 - protein, 110 aa, Mus musculus
EVKLVESGPGLVQPSQSLSITCTVSGFSLTNYDVHWVRQSPGKGLEWLGVIWSGGNTDYNAAFISRLSITKDNSKSQVFFKMNSLQTKDTAIYSCARNHGDGYFNWYFDV

SEQ ID NO: 393 - protein, 110 aa, Mus musculus
EVQLVESGPGLVQPSQSLSITCTVSGFSLTNYDVHWVRQSPGKGLEWLGVIWNYGNTDYNAAFISRLSIRKDSSKSQVFFTMSSLQTPDTAIYYCARNHGDGYYNWYFDV

SEQ ID NO: 394 - protein, 104 aa, Mus musculus
EVQLVESGGGLVKPGGSLKLSCAASEFTFSNYAMSWVRQTPEKGLEWVATISSGGSYTYYSDSVKGRFTISRDNVKNTLYLQMSSLRSEDTAMYYCVRHGYFDV

SEQ ID NO: 395 - protein, 109 aa, Mus musculus
EVQLVESGGGLVKPGGSLKLSCAASGFTFSSYAMSWVRQTPEKRLEWVASISSGGSTYYPDTVKGRFTISRDNARNILYLQMSSLRSEDTAMYYCVRYYYGIRYWYFDV

SEQ ID NO: 396 - protein, 109 aa, Mus musculus
QVQLQESGGVLVKPGGSLKLSCAASGFTFSNYAMSWVRQTPEKRLEWVASISSGGNTFYPDNVKGRFTISRDNSRNILYLQMTSLRSEDSAMYYCVRYYYGVTYWYFDV

SEQ ID NO: 397 - protein, 113 aa, Mus musculus
QVQLKESGGGLVQPGGSLKLSCAASGFTFSNYGMSWVRQIPDKRLELVAAINSNGDITYDPDSVKGRFTISRDNANNSLFLQMRSLKSEDTAMYYCARGTAWFTYWGQGTLVT

SEQ ID NO: 398 - protein, 108 aa, Mus musculus
EVQLQESGPELVKPGASMKISCKASGYSFTGYTMNWVKQSHGENLEWIGLINPYNGGTRYNQKFKDKATLTVNKSSSTAYMELLSLTSEDSAVYYCARDGDDGWDIDV

SEQ ID NO: 399 - protein, 109 aa, Mus musculus
QVQLQESGPELVKPGASMKISCKASGYSFTGYTMNWVKQSHGKNLEWIGLINPYNGGTMYNQKFKGKATLTVDKSSNTAYMELLSLTSEDSAVYYCARDNYGSSPYFDY

SEQ ID NO: 400 - protein, 103 aa, Mus musculus
EVQLQQPEAELVRPGASVKLSCKASGYTFTSYRMNWVKQRPEEGLEWIGRIDPYDSGTHYNQKFKDKAILTVDKSSSIAYMQLSSLTSEDSAVYYCAFYDGAY

SEQ ID NO: 401 - protein, 109 aa, Mus musculus
EVQLQESGPELVKPEASVKISCKASGYSFTGYTMNWVKQSHGKNLEWIGLINPYNGGTTYNQKFKGKATLTVDTSSSTAFMELLSLTSEDSAVYYCARDYYGSSPDFDY

SEQ ID NO: 402 - protein, 107 aa, Mus musculus
EVKLVESGPELKKPGETVKISCKASGYIFANYGMNWVKQAPGKGLKWMGWINTYTGEPTYADDFKGRFAFSLETSASTARLQINNLKKEDTATYFCARRGTYWHFDV

SEQ ID NO: 403 - protein, 110 aa, Mus musculus
QVQLKESGPELEKPGASVRISCKASGYSFTAYNINWVTQRDGKSLEWIGSIDPYYGDTKYNQKFKDKATLTVDKSSSTAHMQVKSLTSEDSAIYYCARRMITMGDWYFDV

SEQ ID NO: 404 - protein, 106 aa, Mus musculus
QVQLQESGPELKKPGETVKISCKASGYTFTNYGMNWVKQAPGKGLKWMGWINTYTGEPTYADDFKGRFALSLEASVSTAYLQINNLKNEDTATYFCARRSTLVFDY

SEQ ID NO: 405 - protein, 108 aa, Mus musculus
QVQLKESGAELVRPGVSVKISCKGSGYTFTNFAIHWVKQSHAKSLEWIGLISSNSGDVSYNQKFKGKATMTVDKSSSTAYMELARLTSEDSAIYYCARHYGAHNYFDY

SEQ ID NO: 406 - protein, 108 aa, Mus musculus
QVTLKESGAELVRPGASVKLSCKASGYSFTSYWMNWVKQRPGQGLEWIGMIHPSDSETRLNQKFKDKATLTVDKSSSTAYMQLSSPTSEDSAVYYCARWGDHDDAMDF

SEQ ID NO: 407 - protein, 103 aa, Mus musculus
QVQLKESGADLVKPGASVKLSCTASGFNIKDTYMNWVKERPEQGLEWIGRIDPANGNTKYDPKFQGKATITADTSSNTGYLQLSSLTSEDTAVYYCASYDPDY

SEQ ID NO: 408 - protein, 108 aa, Mus musculus
EVQLVESGPELVRPGASMRISCKASGYSFTDYTMNWVKQSHGKNLEWIGLINPYNGGTRNNQKFKGKATLTVDKSSSTAYMELLSLTSEDSAVYYCARDTTATYYFDY

SEQ ID NO: 409 - protein, 106 aa, Mus musculus
EVQLQQSGPELVKPGTSVKISCKASGYTFTDYYINWVKQKPGQGLEWIGWIYPGGGNTRYIERFKGKATLTVDTSSSTAYMQLSSLTSEDTAVYFCARNGYWYFDV

SEQ ID NO: 410 - protein, 107 aa, Mus musculus
EVQLQQSGPELKKPGETVKISCKASGYNFTNYGMNWVKQAPGKGLKWMGWINTYTGEPTYADDFKGRFAFSLETSASTVYLRINNLKNEDSSTFFCARRGSYWHFDV

SEQ ID NO: 411 - protein, 109 aa, Mus musculus
EVQLQQPGPELVKPGASMKISCKASGYSFTDYTMNWVKQSHGKNLEWIGLINPYNGGTMYNQKFKDKATLTVDKSSNTAYMELLSLTSEDSAVYYCARDNYGSSPDFDY

SEQ ID NO: 412 - protein, 106 aa, Mus musculus
EVKLVESGPELVKPGTSVKISCKASGYTFTDYYINWVKQRPGQGLEWIGWIYPGGGNTRYIERFKGKATLTVDTSSSTAYMQLSSLTSEDTAVYFCARNGYWYFDV

SEQ ID NO: 413 - protein, 106 aa, Mus musculus
EVQLKESGPELKKPGETVKISCMTSGYMFTNHGMNWVKQAPGKGLKWMGWINTYTGEPTYGDGFKGRFVFSLETSASTAYLQINNLKNEDTATYFCARRVATYFDV

SEQ ID NO: 414 - protein, 108 aa, Mus musculus
EVQLKESGAELVRPGGSVKISCKGSGYTFTNFAIHWVKQSHIKTLEWIGLISTSSGDVSYNQKFKDKATMTVDKSSSTAYMELARLTSEDSAIYYCARHYGANNYFDY

SEQ ID NO: 415 - protein, 106 aa, Mus musculus
EVKLVESGPELKKPGETVKISCKASGYMFTNYGMNWVKQAPGKGLKWMGWINTYTGEPTYVEDFKGRFAFSLETSANTAYLQINNLKNEDTATYFCTRRSHITLDY

SEQ ID NO: 416 - protein, 108 aa, Mus musculus
EVQLQESGAELVRPGVSVKISCKGSGYTFTNFAIHWVKQSHAKSLEWIGLISSNSGDVSYNQKFKGKATMTVDKSSSTAYMELARLTSEDSAIYYCARHYGAHNYFDY

SEQ ID NO: 417 - protein, 110 aa, Mus musculus
EVQLKESGAELVKPGASVKLSCTASGFNIKDTYMHWVKQRPEQGLEWIGRIDPANGNTKYDPKFQGKATITADTSSNTAYLQLSSLTSEDTAVYYCAREENYYGTYYFDY

SEQ ID NO: 418 - protein, 109 aa, Mus musculus
QVQLKESGPELVKPGASMKISCKASGYSFTGYTMNWVKQSHGKNLEWIGLINPYNGGTMYNQKFKGKATLTVDKSSNTAYMELLSLTSEDSAVYYCARDNYGSSPYFDY

SEQ ID NO: 419 - protein, 106 aa, Mus musculus
KVQLQQSGPELKKPGETVKISCKASGYTFTNYGMNWVKQAPGKGLKWMGWINTYTGEPTYADDFKGRFAFSLETSARTAYLQINNLKNEDSATYFCARRRDGNFDY

SEQ ID NO: 420 - protein, 110 aa, Mus musculus
EVKLVESGPELVKPGASVRISCKSSGNTFTNFYLHWMKQRPGQGLEWIGCIYPGNVKTKYSARFKGKAILTADKSSSTVFMQLSNLTSEDSAVYFCAKEGDYDGTAYFDY

SEQ ID NO: 421 - protein, 106 aa, Mus musculus
QVTLKESGPELKKPGETVKISCRASGYIFTNYGMNWVKQAPGKGLKWMGWINTYTGEPTYADDFKGRFAFSLETSASTAHLQINNLKNEDTAIYFCARRRTTAFDY

SEQ ID NO: 422 - protein, 109 aa, Mus musculus
EVKLVESGPELVKPEASVKISCKASGYSFTGYTMNWVKQSHGKNLEWIGLINPYNGGTTYNQKFKGKATLTVDTSSSTAFMELLSLTSEDSAVYYCARDYYGSSPDFDY

SEQ ID NO: 423 - protein, 109 aa, Mus musculus
EVKLVESGAELVRSGASVKLSCAASGFNIKDYYIHWVKQRPEQGLEWIGWIDPENGRTEYAPKFQGKATMTADTSSNTAYLQLSSLTSEDTAVYYCNNGNYVRHYYFDY

SEQ ID NO: 424 - protein, 106 aa, Mus musculus
QVQLQQPGPELKKPGETVKISCKASGYTFINYGMNWVKQAPGKGLKWMGWINTYTGEPTYADDFKGRFAFSLETSASTAYLQINNLEHEDMAVYFCTRRREITFDY

SEQ ID NO: 425 - protein, 108 aa, Mus musculus
QVQLQQSGAELVRPGVSVKISCKGSGYTFTNFAIHWVKQSHAKSLEWIGLISSNSGDVSYNQKFKGKATMTVDKSSSTAYMELARLTSEDSAIYYCARHYGAHNYFDY

SEQ ID NO: 426 - protein, 110 aa, Mus musculus
QVQLKESGPELEKPGASVKISCKASGYSFTAYSMNWVKQNNGMSLEWIGSIDPYYGDTKYAQKFKGKATLTVDKASSTAYLQLKSLTSEDSAVYYCARRMITTGDWYFDV

SEQ ID NO: 427 - protein, 105 aa, Mus musculus
QVQLQQPGAELARPGASVMLSCKASGYTFTDYFINWVKQRTGQGLDWIGEIYPGSSNTYYNEKFKGKATLTADESSSTAYMRLSSLTSEDSAVFCARSGISPFTY

SEQ ID NO: 428 - protein, 105 aa, Mus musculus
QVQLKESGADLVKPGASVKMSCKTSGYIFTGYNIHWVKQTPGQGLVWIGAVYPGNGDTSYNQNFKAKATLTADISSTTAYMQLSSLTSEDSAIYYCAKYDRGFAS

SEQ ID NO: 429 - DNA, 291 bp, Mus musculus
aacattgtgctgacccagtctacatcctccctgtctgcctctctgggagacagagtcaccatcagttgcagtgcaagtcagggcattagaaattatttaaactggtatcagcagaaaccagatggaactgttaaactcctgatctatcacacatcaactttacactcaggagtcccatcaaggttcagtggcagtgggtctgggacagattattctctcaccatcagcaacctggaacctgaagatattgccacttactattgtcagcagtatagtaaccttccgctcacg

SEQ ID NO: 430 - DNA, 294 bp, Mus musculus
gacattgtgctgacccagtctccagcaatcatgtctgcatctccaggggagcaggtcaccatgacctgcagggccacctcaagtataagttccagttacttgcactggtaccagcagaagtccggtgcctcccccaaactctggatttatggcacatccaacttggcttctggagtccctactcgcttcagtggcagtgggtctgggacctcttactctctcacaatcagcagtgtggaggctgaagatgctgccacttattactgccagcagtacagtgattacccactcacg

SEQ ID NO: 431 - DNA, 291 bp, Mus musculus
gacattgtgctgacccaatctccagcctccctatctgcatctgtgggagaatctgtcaccatcacatgtcggccaagtgaaaatatttacggttatttcgcatggtatcagcagagacagggaaaatctcctcagctcctggtctataatgcaaaaaccttagcagaaggtgtgccatcaaggttcagtggcagtggatcaggcacacatttttctctgaagatcaacagcctacagcctgaagattttgggacttattactgtcaacatcattatggtactccattcacg

SEQ ID NO: 432 - DNA, 291 bp, Mus musculus
gacattgtgctgactcagtctccagcctccctggctgcatctgtgggagaaactgtcaccatcacatgtcgagcaagtgagaacatttactacaatttagcatggtatcagcagaagcaagggaaatctcctcagctcctgatctataatgcaaacagcttggaaggtggtgtcccatcgaggttcagtggcagtggatctgggacacagtattctatgaagatcaacagcatgcagcctgaagacaccgcaacttatttctgtaaacaggcttatgacgttccgtggacg

SEQ ID NO: 433 - DNA, 288 bp, Mus musculus
gaaattgtgctgacccagtctccagcaatcatgtctgcatctccaggggagaaggtcaccatgacctgcagtgccagctcaagtttaagttacatgtattggtaccagcagaagccaggatcctcccccagactcctgatttatgacacatccaacctggcttctggagtcccttttcgcttcagtggcagtgggtctgggacctcttactctctcacaatcagccgaatggaggctgaagatgctgccacttattactgccagcagtggagtagtttcccaccgaca

SEQ ID NO: 434 - DNA, 294 bp, Mus musculus
gaaattgttctgacccagtctccagcaatcatgtctgcatctccaggggagcaggtcaccatgacctgcagggccacctcaagtataagttccagttacttgcactggtaccagcagaagtccggtgcctcccccaaactctggatttatggcacatccaacttggcttctggagtccctactcgcttcagtggcggtgggtctgggacctcttactctctcacaatcagccgaatggaggctgaagatgctgccacttattactgccagcaaaggagttatttcccgttcacg

SEQ ID NO: 435 - DNA, 309 bp, Mus musculus
aacattgtgatgacacagtctccatcctccctagctgtgtcagttggagagaaggttactatgagctgcaagtccagtcagagccttttatatagtagcgatcaaaagaactacttggcctggtaccagctgaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattataactatccgctcacg

SEQ ID NO: 436 - DNA, 291 bp, Mus musculus
aacattgtgctgactcagtctccagcctccctggctgcatctgtgggagaaactgtcaccatcacatgtcgagcaagtgagaacatttactacaatttagcatggtatcagcagaagcaagggaaatctcctcagctcctgatctataatgcaaacagcttggaagatggtgtcccatcgaggttcagtggcagtggatctgggacacagtattctatgaagatcaacagcatgcagcctgaagataccgcaacttatttctgtaaacaggtttatgacgttccattcacg

SEQ ID NO: 437 - DNA, 291 bp, Mus musculus
cacattgttctgacccaatctccagccatcctgtctgtgagtccaggggagagagtcagtttctcctgcagggccagtcagaccattgccacaagcataaactggtatcagcaaagaacaaatggttctccaaggcttctcataaagaatgcttctgagtctatctctgggatcccttccaggtttagtggcagtggatcagggacagattttactcttaccatcaacagtgtggagtctgaagatattgcagattattactgtcaacaaagtaatagctggccactcacg

SEQ ID NO: 438 - DNA, 291 bp, Mus musculus
cacattgttctgacccaatctccatcttccctgtctgcctctctgggagacagagtcaccatcagttgcagggcaagtcaggacattagtacttatttaaactggtatcagcagaaaccggatggaactgttaaactcctgatcttctacacatcacgattacacgcaggagtcccatcaaggttcagtggcagtgggtctggaacacatcattctctcaccattagcaacctggaacaagaagatattgccacttacttttgccaacagggtaattcgcttccgttcacg

SEQ ID NO: 439 - DNA, 288 bp, Mus musculus
gatattgtgatgacacagtctccatcctcactgtctgaatctctgggaggcaaagtcaccatcacatgcaaggcaagtcaagacattaacaactatatagcttggtaccaacacaagcctggaaaaggtcctaggctgctcatacattacacatctacattgctgccaggcatcccatcaaggttcagtggaagtgggtctgggacagattattccttcagcatcagcaacctggagcctgaagatattgcaacttattattgtctacagtatgataatctgtggacg

SEQ ID NO: 440 - DNA, 288 bp, Mus musculus
gatgttgtgctgacccaatctccagcaatcctgtctgcatcgccaggggagaaggtcacaatgacttgcagggccatctcaagtgtaagttacatgcactggtaccagcagaagccaggatcatcccccaaaccctggatttatgccacttccaacctggcttctggagtccctgctcgcttcagtggcagtgggtctgggacctcttactctctcacaatcagcagagtggaggctgaagatgctgccacttattactgccagcagtggagtagtaacccacggacg

SEQ ID NO: 441 - DNA, 291 bp, Mus musculus
aacattgtgctgacacagtctacatcctccctgtctgcctctctgggagacagagtcaccatcaattgcagtgcaagtcagggcattagcaatcatttaaactggtttcagcagaaatcagatggaactgttaaactcctgatctatttcacatcaagtttacactcgggagtcccttcaaggttcagtggcagttggtctgggacagattattctctcaccatcagcaacctggaacctgaagatattgccgcttactattgtcagcagtatagtaaccttccgtacacg

SEQ ID NO: 442 - DNA, 291 bp, Mus musculus
aacattgtgctgacacagtctccagccatcctgtctgtgagtccaggggagagagtcagtttctcctgcagggccagtcagaacattggcacaagcatacactggtatcagcaaagaacaaatggttctccgaggtttctcgtaaaggatgcttctgagtctatctctgggatcccttccaggtttagtggcagtggatcagggacagattttactcttaccatcaacaatgtggagtctgaagatattgcagattattactgtcaacaaagtgatagctggccactcacg

SEQ ID NO: 443 - DNA, 291 bp, Mus musculus
aacattgtgctgactcagtctccagcctccctggctgcatctgtgggagaaactgtcaccatcacatgtcgagtaagtgagaacatttactacaatttagcatggtatcagcagaagcaagggaaatctcctcagctcctgatctataatgcaaacagtttggaagatggtgtcccatcgaggttcagtggcagtggatctggaacacagtattctatgaagatcaacagcatgcagcctgaagataccgcaacttatttctgtcaacagacttttgacgttccgtggacg

SEQ ID NO: 444 - DNA, 291 bp, Mus musculus
cacattgtgctcacccaatctccagccatcctatctgtgagtccaggagagagagtcagtttctcctgcagggccagtcagaccattggcacaagcatacactggtatcagcaaagaacaaatggttctccaaggcttctcataaagaatgcttctgagtctatctctgggatcccttccaggtttagtggcagtggatcagggacagattttactcttagcatcaacagtgtggagtctgaagatattgcagattattactgtcaacaaagtaatagctggccactcacg

SEQ ID NO: 445 - DNA, 300 bp, Mus musculus
caaattgttctcacccagtctccagcttctttgcctgcgtctccaggacagagggccaccatctcctgcaaggccagccaaagtgttgattatgatggtgatagttatatgaactggtaccatcagaaaccaggacagccacccaaactcctcatctatgctgcatccaatctcgaatctgggatcccagccaggtttagtggcagtgggtctgggacagacttcaccctcaacatccatcctgtggaggaggaggatgctgcaacctattactgtcagcaaagtaatgaggatccgacg

SEQ ID NO: 446 - DNA, 291 bp, Mus musculus
aacattgtgctgactcagtctacatcctccctgtctgcctctctgggagacagagtcaccatcaattgcagtgcaagtcagggcattagcaatcatttaaactggtttcagcagaaatcagatggaactgttaaactcctgatctatttcacatcaagtttacactcgggagtcccttcaaggttcagtggcagtgggtctgggacagattattctctcaccatcagcaacctggaacctgaagatattgccgcttactattgtcagcagtatagtaaccttccgtacacg

SEQ ID NO: 447 - DNA, 300 bp, Mus musculus
aacattgtgctgacccagtctccagcttctttggctgtgtctctaggacagagggccaccatctcctgcaaggccagccaaagtgttgatcatgatggtgatagttatatgaactggtaccaacagaaaccaggacagtcacccaaactcctcacctatgctgcatccaatctagattctgggatcccagccaggtttagtggcagtgggtctcggacagacttcaccctcaacatccaccctgtggaggaggaggatgctgcaacctattactgtcagcaaaattatgaggatccgacg

SEQ ID NO: 448 - DNA, 291 bp, Mus musculus
gaaattgttctcacccagtctccatcctccctgtctgcctctctgggagacagagtcaccatcagttgcagggcaagtcaggacattagcaattatttaaactggtatcagcggaaaccagatgggactgttaaactcctgatctactacacatcaagattacagtcaggagtcccatcaaggttcagtggcagtgggtctggttcagagtattctctcaccattagcaacctggaccaagaggatattgccacttacttttgtcaacagggtgatgcgcttccgtggacg

SEQ ID NO: 449 - DNA, 291 bp, Mus musculus
gacattgtgctgacacagtctccagtcatcctgtctgtgagcccaggagaaagagtcagtctctcctgcagggccagtcagagcattggcacaagcataaattggtatcagcagagaacagatggttctccaaggcttctcataaagtctgcttctgagtctatgtctgggatcccttccaggtttagtggcagtggatcagggacagattttactcttagcatcaccagtgtggagtctgaagatattgcagattattactgtcaacaaagtaatagctggccgctcacg

SEQ ID NO: 450 - DNA, 294 bp, Mus musculus
gaaattgttctcacccaatctccaacaatcatgtctgcttctccaggggagcaggtcaccatgacctgccggaccaactcaagtgtaagttccagttacttgcactggtaccagcagaagtcaggtgcctcccccaaactctggatttatggcacatccaacttggcttctggagtccctactcgtttcagtggcagtgggtctgggacctcttactctctcacaatcagcagtgtggaggctggagatgctgccacttatttctgccagcagtacagtggttacccactcacg

SEQ ID NO: 451 - DNA, 291 bp, Mus musculus
aacattgtgctgacccagtctccagccatcctgtctgtgagtccaggagagagagtcagtttctcctgcagggccagtcagcgcattggcacaagcatgaactggtatcaacaaagaacaaatggttctccaaggcttctcataaagtctgcttctgagtctatctctgggatcccttccaggtttagtggcagcggttcagggacagattttactcttagcatcaacagtgtggagtctgacgatgttgcagattattactgtcaacaaagtaatagttggccgctcacg

SEQ ID NO: 452 - DNA, 294 bp, Mus musculus
gacattgtgatgacacagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaagctgaagatgctgccacttattactgccaccagtatcatcgttccccgctcacg

SEQ ID NO: 453 - DNA, 291 bp, Mus musculus
aacattgtgctgacccagtctacatcctccctgtctgcctctctgggagacagagtcaccatcagttgcagtgcaagtcagggcattagaaattatttaaactggtatcagcagaaatcagatggaactgttaaactcctgatctatttcacatcaagtttacactcgggagtcccttcaaggttcagtggcagtgggtctgggacagattattctctcaccatcagcaacctggaacctgaagatattgccgcttactattgtcagcagtatagtaaccttccgtacacg

SEQ ID NO: 454 - DNA, 303 bp, Mus musculus
aacattgtgctgactcagtctccagcttctttggctgtatctctagggcagagagccaccatctcctgcagagtcaatgaaagtgttgaatattctggcacaagtttaatgcagtggtaccaacagaaaccaggacagccacccaaactcctcatctctgctgcatccaacgtagaatctggggtccctgccaggtttagtggccgtgggtctgggacagacttcagcctcaacatccatcctgtggaggaggatgatattgcaatgtatttctgtcagcaaagtaggcaggttcctctcacg

SEQ ID NO: 455 - DNA, 300 bp, Mus musculus
gacattgtgctgacacagtctccatcctccctgactgtgacagcaggagagaaggtcactatgagctgcaagtccagtcagagtctcttaaatcaaaagaactacttgacctggtaccagcagaaaacagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatgattatccttacacg

SEQ ID NO: 456 - DNA, 294 bp, Mus musculus
aacattgtgatgactcagtctccagcaatcatgtctgcatctccaggggagcaggtcaccatgacctgcagggccacctcaagtataagttccagttacttgcactggtaccagcagaagtccggtgcctcccccaaactctggatttatggcacatccaacttggcttctggagtccctactcgcttcagtggcagtgggtctgggacctcttactctctcacaatcagcagtatggaggctgaagatgctgccacttattactgccagcagtggagtagtagaccacccacg

SEQ ID NO: 457 - DNA, 291 bp, Mus musculus
aacattgtgatgactcagtctccagccattctgtctgtgagtccaggagaaagagtcagcttctcctgcagggccagtcagagcattggtacaagcataaactggtatcagcaaagaacaaatgcttctccaaggcttctcataaagagtgcttctgagtctatctctgggatcccttccaggtttagtggcagtggatcagggacagattttactcttaacatcaaaaatgtggagtctgaagatattgcagattattactgtcaacaaagtaataggtggccgctcacg

SEQ ID NO: 458 - DNA, 306 bp, Mus musculus
atgtttgtgatgacgcaaactccactctccctgcctgtcagtcttggagatcaagcctccatctcttgcagatctagtcagagccttgtacacagtaatggaaacacctatttacattggtacctacagaagccaggccagtctccaaagctcctgatctacaaagtttccaatcgattttctggggtcccagacaggttcagtggcagtggatcagggacagatttcacactcaagatcagcagagtggaggctgaggatctgggagtttattattgctggcaaaatacacattttcctcagacg

SEQ ID NO: 459 - DNA, 291 bp, Mus musculus
gacattgtgctgacacagtctccagcctccctatctgcatctgtgggagaaactgtcatcatcacgtgtcgagcaagtgagaatatttacagttatttagtatggtatcagcagaaacagggaaaatctcctcagctcctggtctataatgcaaaaaccttagcagaaggtgtgccatcaaggttcagtggcagtggatcaggcacacagttttctctgaagatcaacagcctgcagtctgaagattttgggagttattcctgtcaacatcattatggttatccgtatacg

SEQ ID NO: 460 - DNA, 291 bp, Mus musculus
gacattgtgctgactcagtctacatcctccctgtctgcctctctgggagacagagtcaccatcaattgcagtgcaagtcagggcattagcaatcatttaaactggtttcagcagaaatcagatggaactgttaaactcctgatctatttcacatcaagtttacactcgggagtcccttcaaggttcagtggcagtgggtctgggacagattattctctcaccatcagcaacctggaacctgaagatattgccgcttactattgtcagcagtatagtaaccttccgtacacg

SEQ ID NO: 461 - DNA, 291 bp, Mus musculus
gacattgtgatgacccagtctacatcctccctgtctgcctctctgggagacagagtcaccatcagttgcagtgcaagtcagggcattaacaattatttaaactggtatcagcagaaaccagatggaactgttaaactcctgatctattacacatcaagtttacactcaggagtcccaccaaggttcagtggcagtgggtctgggacagattattctctcaccatcagcaacctggaacctgaagatattgccacttactattgtcagcagtatagtaagattccgtacacg

SEQ ID NO: 462 - DNA, 291 bp, Mus musculus
cacattgtgctgacccaatctcacaaattcatgtccacatcagtaggagacagggtcagcatcacctgcaaggccagtcaggatgtgattactgctgtaacctggtctcaacagaaaccaggacaatctcctaaactactgatttactcggcatcctaccggtacactggagtccctgatcgcttcactggcagtggatctgggacggatttcactttcaccatcagcagtgtacaggctgaagacctggcagtttattactgtcagcaacattatagtactccgtggacg

SEQ ID NO: 463 - DNA, 288 bp, Mus musculus
gacattgttctgacccagtctccagcaatcatgtctgcatctccaggggagaaggtcaccatgacctgcagtgccaactcaagtgtaagttacatgctctggtaccagcagaagtcaggcacctcccccaaaagatggatttatgacacatccaaactgtcttctggagtccctgctcgcttcagtggcagtgggtctgggacctcttactctctcacaatcagcagcatggaggctgaagatgctgccacttattactgccagcagtggagtagtaacccattcacg

SEQ ID NO: 464 - DNA, 291 bp, Mus musculus
aacattgtgatgacccagtctcacagattcatgtccacatcagtaggagacagggtcagcatcacctgcaaggccagtcaggatgtgaggactgatgtagcctggtttcaacagaaaccaggacaatctcctaaactactgatttactcggcatccttccggtacactggagtccctgaccgcttcactggcagtggatctgggacggatttcactctcaccatcagcagtgtgcaggctgaagacctggcagtttattactgtcagcaacattatacttctccgtggacg

SEQ ID NO: 465 - DNA, 288 bp, Mus musculus
aacattgtgctgacacagtctccatcctcactgtctgaatctctgggaggcaaagtcaccatcacatgtaaggcaagtcaagacattaacaagtatatagcttggtaccaatacaagcctggaaaaggtcctaggctgctcatacattacacatctacattacagccaggcatcccatcaaggttcagtggaagtggttctgggagagattattccttcagcatcagcaacctggagcctgaagatattgcaacttattattgtctacagtacgataatctgtggacg

SEQ ID NO: 466 - DNA, 330 bp, Mus musculus
gaagtgaagcttgttgagtcaggacctggcctagtgcagccctcacagagcctgtccatcacctgcacagtctctggtttctcattaactaactatgatgtacactgggttcgccagtctccaggaaagggtctggagtggctgggagtgatttggagtggtggaaacacagactataatgcagctttcatatccagactgagcatcaccaaggacaattccaagagccaagttttctttaaaatgaacagtctgcaaactaaagacacagccatatactcctgtgccagaaatcatggtgatggttacttcaactggtacttcgatgtc

SEQ ID NO: 467 - DNA, 330 bp, Mus musculus
gaggtgcagctggttgagtcaggacctggcctagtgcagccctcacagagcctgtccatcacctgcacagtctctggtttctcattaactaactatgatgtacactgggttcgccagtctccaggaaagggtctggagtggctgggagtgatatggaattatggaaacacagactataatgcagctttcatatccagactgagcatcaggaaggacagttccaagagccaagttttctttacaatgagcagtctgcaaactcctgacacagccatatattactgtgccagaaatcatggtgatggttactataactggtacttcgatgtc

SEQ ID NO: 468 - DNA, 312 bp, Mus musculus
gaggtgcagcttgtggagtctgggggaggcttagtgaagcctggagggtccctgaaactctcctgtgcagcctctgaattcactttcagtaactatgccatgtcttgggttcgccagactccggagaagggcctggagtgggtcgcaaccattagtagtggtggtagttacacctactattcagacagtgtgaagggtcgattcaccatctccagagacaatgtcaagaacaccctgtatctgcaaatgagcagtctgaggtctgaggacacggccatgtattactgtgtaagacatggatacttcgatgtc

SEQ ID NO: 469 - DNA, 327 bp, Mus musculus
gaagtgcagcttgttgagtctgggggaggcttagtgaagcctggagggtccctgaaactctcctgtgcagcctctggattcactttcagtagctatgccatgtcttgggttcgccagactccagagaagaggctggagtgggtcgcatccattagtagtggtggtagcacctactatccagacactgtgaagggccgattcaccatctccagagataatgccaggaacatcctgtacctgcaaatgagcagtctgaggtctgaggacacggccatgtattactgtgtccgttattactacggtattaggtactggtacttcgatgtc

SEQ ID NO: 470 - DNA, 327 bp, Mus musculus
caggtgcagcttcaggagtctgggggagtcttagtgaagcctggagggtccctgaaactctcctgtgcagcctctggattcactttcagtaactatgccatgtcttgggttcgccagactccagagaagaggctggagtgggtcgcgtccattagtagtggtggtaacaccttttatccagacaatgtgaagggccgattcaccatctccagagataattccaggaacatcctgtacctgcaaatgaccagtctgaggtctgaggactcggccatgtattactgtgtccgttattactacggtgttacctactggtacttcgatgtc

SEQ ID NO: 471 - DNA, 341 bp, Mus musculus
caggtgcagcttaaggagtctgggggaggcttagtgcagcctggagggtccctgaaactctcctgtgcagcctctggattcactttcagtaactatggcatgtcttgggttcgccagattccagacaagaggctggaattggtcgcagccattaatagtaatggtgatattacctatgatccagacagtgtgaagggccgattcaccatctccagagacaatgccaacaactccctgttcctgcaaatgagaagtctgaagtctgaggacacagccatgtattactgtgcaagaggaactgcctggtttacttactggggccaagggactctggtcactgt

SEQ ID NO: 472 - DNA, 324 bp, Mus musculus
gaggtgcagcttcaggagtctggacctgagctggtgaagcctggagcttcaatgaagatatcctgcaaggcttctggttactcattcactggctacaccatgaactgggtgaagcagagccatggagagaaccttgagtggattggacttattaatccttacaatggtggtactagatacaaccagaagttcaaggacaaggccacattaactgtaaacaagtcatccagcacagcctacatggagctcctcagtctgacatctgaggactctgcagtctattactgtgcaagagatggggatgatggttgggacatcgatgtc

SEQ ID NO: 473 - DNA, 327 bp, Mus musculus
caggtgcagcttcaggagtctggacctgagctggtgaagcctggagcctcaatgaagatatcctgcaaggcttcaggttattcattcaccggctacaccatgaactgggtgaagcagagccatggaaagaaccttgagtggattggacttattaatccttacaatggtggtactatgtacaaccagaagttcaagggcaaggccacattaactgtagacaagtcatccaatacagcctacatggagctcctcagtctgacatctgaggactctgcagtctattactgtgcaagagataactacggtagtagcccatactttgactac

SEQ ID NO: 474 - DNA, 309 bp, Mus musculus
gaggtccaactgcaacagcctgaggctgagctggtgaggcctggggcttcagtgaagctgtcctgcaaggcttctggctacacgttcaccagctacaggatgaactgggttaagcagaggcctgaggaaggccttgagtggattggaaggattgatccttacgatagtggaactcactacaatcaaaagttcaaggacaaggccatattgactgtagacaaatcctccagtatagcctacatgcaactcagcagcctgacatctgaggactctgcggtctattactgtgccttctatgatggggcttac

SEQ ID NO: 475 - DNA, 327 bp, Mus musculus
gaggtgcagctgcaggagtctggacctgagctggtgaagcctgaagcttcagtgaagatatcctgcaaggcttctggttactcattcactggctacaccatgaactgggtgaagcagagccatggaaagaaccttgaatggattggacttattaatccttacaatggtggtactacctacaaccagaagttcaagggcaaggccacattaactgtagacacgtcatccagcacagccttcatggagctcctcagtctgacatctgaagactctgcagtctattactgtgcaagagattactacggtagtagtccagactttgactac

SEQ ID NO: 476 - DNA, 321 bp, Mus musculus
gaggtgaagcttgttgagtctggacctgagctgaagaagcctggagagacagtcaagatctcctgcaaggcttctgggtatattttcgcaaactatggcatgaactgggtgaagcaggctccaggaaagggtttaaagtggatgggctggataaacacctacactggagagccaacatatgctgatgacttcaagggacggtttgccttctctttggaaacctctgccagcactgcccgtttgcagatcaacaacctcaaaaaagaggacacggctacatatttctgtgcaagaagggggacttactggcacttcgatgtc

SEQ ID NO: 477 - DNA, 330 bp, Mus musculus
caggtgcagctgaaggagtctggacctgaactggagaagcctggcgcttcagtgaggatttcctgcaaggcttctggttattcattcactgcctacaacattaactgggtgacgcagcgcgatggaaagagccttgagtggattggaagtattgatccttactatggtgatactaaatacaaccagaagttcaaggacaaggccacgttgactgtagacaaatcctccagcacagcccacatgcaggtcaagagcctcacatctgaggactctgcaatctattactgtgcaagaaggatgattacgatgggagactggtatttcgatgtc

SEQ ID NO: 478 - DNA, 318 bp, Mus musculus
caggtgcagctgcaggagtctggacctgagctgaaaaagcctggagagacagtcaagatctcctgcaaggcttctggttataccttcacaaactatggaatgaactgggtgaagcaggctccaggaaagggtttaaagtggatgggctggataaacacttacactggagagccaacatatgctgatgacttcaagggacggtttgccctctctttggaagcctctgtcagcactgcctatttgcagatcaacaacctcaaaaatgaagacacggctacatatttctgtgcaagacgctctacgctcgtctttgactac

SEQ ID NO: 479 - DNA, 324 bp, Mus musculus
caggtgcagcttaaggagtctggggctgaactggtgaggcctggggtctcagtgaagatttcctgcaagggttctggctacacattcactaattttgctattcactgggtgaaacagagtcatgcaaagagtctagagtggattggacttattagttctaactctggtgatgttagctacaaccagaagttcaagggcaaggccacaatgactgtagacaaatcctccagcacagcctatatggaacttgccagactgacatctgaggattctgccatctattattgtgcaagacactatggtgcccacaactattttgactat

SEQ ID NO: 480 - DNA, 324 bp, Mus musculus
caggttactctgaaagagtctggggctgagctggtgaggcctggagcttcagtgaagctgtcctgcaaggcttctggctactccttcaccagttactggatgaactgggtgaaacagaggcctggacaaggccttgaatggattggcatgattcatccttccgatagtgaaactaggttaaatcagaagttcaaggacaaggccacattgactgtagacaagtcctccagcacagcctacatgcaactcagcagcccgacatctgaggactctgcggtctattactgtgcaagatggggggatcacgacgatgctatggacttc

SEQ ID NO: 481 - DNA, 309 bp, Mus musculus
caggtgcagctgaaggagtctggggcagaccttgtgaagccaggggcctcagtcaagttgtcctgcacagcttctggcttcaacattaaagacacctatatgaactgggtgaaggagaggcctgaacagggcctggagtggattggaaggattgatcctgcgaatggtaatactaaatatgacccgaagttccagggcaaggccactataacagcagacacatcctccaatacaggctacctgcagctcagcagcctgacatctgaggacactgccgtctattactgtgctagttatgatcctgactac

SEQ ID NO: 482 - DNA, 324 bp, Mus musculus
gaggtgcagctggttgagtctggacctgagctggtgaggcctggagcttcaatgaggatatcctgcaaggcttctggttactcattcactgactacaccatgaactgggtgaagcagagccatggaaagaaccttgagtggattggacttattaatccttacaatggtggtactaggaacaaccagaagttcaagggcaaggccacattaactgtagacaagtcatccagcacagcctacatggagctcctcagtctgacatctgaggactctgcagtctattactgtgcaagagatactacggcgacgtactactttgactac

SEQ ID NO: 483 - DNA, 318 bp, Mus musculus
gaggtccaactgcaacagtctggacctgagctggtgaagcctgggacttcagtgaagatatcctgcaaggcttctggctacaccttcactgactactatataaactgggtgaagcagaagcctggacagggacttgagtggattggatggatttatcctggaggcggtaatactaggtacattgagaggttcaagggcaaggccacattgactgtagacacatcctccagcacagcctacatgcagctcagcagcctaacatctgaggacactgctgtctatttctgtgcaagaaacggctactggtacttcgatgtc

SEQ ID NO: 484 - DNA, 321 bp, Mus musculus
gaagtccagctgcaacagtctggacctgagctgaagaagcctggagagacagtcaagatctcctgcaaggcttctgggtataacttcacaaactatggaatgaactgggtgaagcaggctccaggaaagggtttaaagtggatgggctggattaacacctacactggagagccaacatatgctgatgacttcaagggacggtttgccttctctttggaaacctctgccagcactgtctatttgcggatcaacaacctcaaaaatgaggactcgtctacatttttctgtgcaagaagggggtcttactggcacttcgatgtc

SEQ ID NO: 485 - DNA, 327 bp, Mus musculus
gaggtccaactgcaacagcctggacctgagctggtgaagcctggagcctcaatgaagatatcctgcaaggcttcaggttactcattcactgactacaccatgaactgggtgaaacagagccatggaaagaaccttgagtggattggacttattaatccttacaatggtggtactatgtacaaccagaagttcaaggacaaggccacattaactgtagacaagtcatccaatacagcctacatggagctcctcagtctgacttctgaggactctgcagtctattactgtgcaagagataactacggtagtagtccagactttgactac

SEQ ID NO: 486 - DNA, 318 bp, Mus musculus
gaagtgaagcttgtggagtctggacctgagctggtgaagcctgggacttcagtgaagatatcctgcaaggcttctggctacaccttcactgactactatataaactgggtgaagcagaggcctggacagggacttgagtggattggttggatttatcctggaggcggtaatactaggtacattgagaggttcaagggcaaggccacattgactgtagacacatcctccagcacagcctacatgcagctcagcagcctaacatctgaggacactgctgtctatttctgtgcaagaaacggctactggtacttcgatgtc

SEQ ID NO: 487 - DNA, 318 bp, Mus musculus
gaggtgcagcttaaggagtctggacctgagctgaagaagcctggagagacagtcaagatctcctgcatgacttctgggtatatgttcacaaaccatggaatgaactgggtgaaacaggctccaggaaagggtttaaagtggatgggctggataaacacctacactggagagccaacatatggtgatggcttcaagggacggtttgtcttctctttggaaacctctgccagcactgcctatttgcagatcaacaacctcaaaaatgaggacacggctacatatttctgtgcaagacgtgttgcgacgtacttcgatgtc

SEQ ID NO: 488 - DNA, 324 bp, Mus musculus
gaggtgcagcttaaggagtctggggctgagctggtgaggcctgggggctcagtgaagatttcctgcaagggttctggctacacattcactaattttgctattcactgggtgaaacaaagtcatataaagactctagagtggattggtcttattagtacttcctctggtgatgttagctacaaccagaagttcaaggacaaggccacaatgactgtagacaaatcctccagcactgcctatatggagcttgccagactgacatctgaggattctgccatctattactgtgcaagacactatggtgccaacaactattttgactat

SEQ ID NO: 489 - DNA, 318 bp, Mus musculus
gaggtgaagcttgttgagtctggacctgagttgaagaagcctggagagacagtcaagatctcctgcaaggcttctgggtatatgttcacaaactatggaatgaattgggtgaagcaggctccaggaaagggtttaaagtggatgggctggataaacacctacactggagagccaacatatgttgaagacttcaagggacggtttgccttctctttggaaacctctgccaacactgcctatttgcagatcaacaacctcaaaaatgaggacacggctacatatttttgtacacgaaggagtcatattaccttggactac

SEQ ID NO: 490 - DNA, 324 bp, Mus musculus
gaggtgcagcttcaggagtctggggctgaactggtgaggcctggggtctcagtgaagatttcctgcaagggttctggctacacattcactaattttgctattcactgggtgaaacagagtcatgcaaagagtctagagtggattggacttattagttctaactctggtgatgttagctacaaccagaagttcaagggcaaggccacaatgactgtagacaaatcctccagcacagcctatatggaacttgccagactgacatctgaggattctgccatctattattgtgcaagacactatggtgcccacaactattttgactat

SEQ ID NO: 491 - DNA, 330 bp, Mus musculus
gaggtgcagcttaaggagtctggggcagagcttgtgaagccaggggcctcagtcaagttgtcctgcacagcttctggcttcaacattaaagacacctatatgcactgggtgaaacagaggcctgaacagggcctggagtggattggaaggattgatcctgcgaatggtaatactaaatatgacccgaagttccagggcaaggccactataacagcagacacatcctccaacacagcctacctgcagctcagcagcctgacatctgaggacactgccgtctattactgtgctagagaggagaattactacggtacctactactttgactac

SEQ ID NO: 492 - DNA, 327 bp, Mus musculus
caggtgcagctgaaggagtctggacctgagctggtgaagcctggagcctcaatgaagatatcctgcaaggcttcaggttattcattcaccggctacaccatgaactgggtgaagcagagccatggaaagaaccttgagtggattggacttattaatccttacaatggtggtactatgtacaaccagaagttcaagggcaaggccacattaactgtagacaagtcatccaatacagcctacatggagctcctcagtctgacatctgaggactctgcagtctattactgtgcaagagataactacggtagtagcccatactttgactac

SEQ ID NO: 493 - DNA, 318 bp, Mus musculus
aaggtccagctgcaacagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagcctacatatgctgatgacttcaagggacggtttgccttctctttggaaacctctgccagaactgcctatttgcagatcaacaacctcaaaaatgaggactcggctacatatttctgtgcaagaaggcgggacggaaactttgactac

SEQ ID NO: 494 - DNA, 330 bp, Mus musculus
gaagtgaagcttgttgagtctggacctgagctggtgaagcctggggcttcagtgaggatatcttgcaagtcctctggcaacaccttcacaaacttctatttacactggatgaaacagaggcctggacagggacttgagtggattggatgtatttatcctggaaacgttaagactaaatacagtgcgaggttcaagggcaaggccatactgactgcggacaaatcctccagcacagtcttcatgcagctcagcaacctgacctctgaggactctgcggtctatttctgtgcaaaggagggagattacgacgggacggcctactttgattac

SEQ ID NO: 495 - DNA, 318 bp, Mus musculus
caggttactctgaaagagtctggacctgaactgaagaagcctggagagacagtcaagatctcctgcagggcttctgggtatatcttcacaaactatggaatgaactgggtgaagcaggctccaggaaagggtttaaagtggatgggctggataaacacctacactggagagccaacatatgctgatgacttcaagggacgatttgccttctctttggaaacctctgccagcactgcccatttgcagatcaacaacctcaaaaatgaggacacggctatatatttctgtgcaagacggaggactacggcctttgactac

SEQ ID NO: 496 - DNA, 327 bp, Mus musculus
gaagtgaagcttgtggagtctggacctgagctggtgaagcctgaagcttcagtgaagatatcctgcaaggcttctggttactcattcactggctacaccatgaactgggtgaagcagagccatggaaagaaccttgaatggattggacttattaatccttacaatggtggtactacctacaaccagaagttcaagggcaaggccacattaactgtagacacgtcatccagcacagccttcatggagctcctcagtctgacatctgaagactctgcagtctattactgtgcaagagattactacggtagtagtccagactttgactac

SEQ ID NO: 497 - DNA, 327 bp, Mus musculus
gaggtgaagctggtggagtctggggcagagcttgtgaggtcaggggcctcagtcaaattgtcctgcgcagcttctggcttcaacattaaagactactatatacactgggtaaaacagaggcctgaacagggcctggagtggattggatggattgatcctgagaatggtcgtactgaatatgccccgaagttccagggcaaggccactatgactgcagacacatcctccaacacagcctacctgcagctcagcagcctgacatctgaggacactgccgtctattactgtaataatggtaactacgtcagacactactactttgactac

SEQ ID NO: 498 - DNA, 318 bp, Mus musculus
caggtccagctgcaacagcctggacctgagctgaagaagcctggagagacagtcaagatctcctgcaaggcttctgggtataccttcataaattatggaatgaactgggtgaagcaggctccaggaaagggtttaaagtggatgggctggataaacacctacactggagagccaacatatgctgatgacttcaagggacggtttgccttctctttggaaacctctgccagcactgcctatttgcagatcaacaacctcgaacatgaggacatggctgtatatttctgtacaagaagaagagaaataacctttgactac

SEQ ID NO: 499 - DNA, 324 bp, Mus musculus
caggtccagctgcaacagtctggggctgaactggtgaggccaggggtctcagtgaagatttcctgcaagggttctggctacacattcactaattttgctattcactgggtgaaacagagtcatgcaaagagtctagagtggattggacttattagttctaactctggtgatgttagctacaaccagaagttcaagggcaaggccacaatgactgtagacaaatcctccagcacagcctatatggaacttgccagactgacatctgaggattctgccatctattattgtgcaagacactatggtgcccacaactattttgactat

SEQ ID NO: 500 - DNA, 330 bp, Mus musculus
caggtgcagctgaaggagtctggacctgagctggagaagcctggcgcttcagtgaagatatcctgcaaggcttctggttactcattcactgcctacagcatgaactgggtgaagcagaacaatggaatgagccttgagtggattggaagtattgatccttattatggtgatactaagtacgcccaaaagttcaagggcaaggccacattgactgtggacaaagcctccagcacagcctacttgcagctcaagagcctgacatctgaagactctgcagtctattactgtgcaagaaggatgattacgacgggagactggtacttcgatgtc

SEQ ID NO: 501 - DNA, 318 bp, Mus musculus
caggtccaactgcaacagcctggagctgagctggcgaggcccggggcttcagtgatgctgtcctgcaaggcttctggctacaccttcactgactactttataaactgggtgaagcagaggactggacagggccttgactggattggagagatttatcctggaagtagtaatacttactacaatgaaaagttcaagggcaaggccacactgactgcagacgaatcctccagcacagcctacatgcggctcagcagcctgacatctgaggactctgcagtctagttctgtgcaagatcggggatttcgccctttacttac

SEQ ID NO: 502 - DNA, 315 bp, Mus musculus
caggtgcagcttaaggagtctggggctgacctggtgaagcctggggcctcagtgaagatgtcctgcaagacttctggctacatttttaccggttacaatatacactgggtcaaacagacgcctggacagggcctggtttggattggagctgtttatccaggaaatggtgatacttcctacaatcagaatttcaaagccaaggccacattgactgcagacatctcctccaccacagcctacatgcagctcagcagcctgacatctgaggactctgcgatctattactgtgcaaaatatgaccgggggtttgcttcc

SEQ ID NO: 503 - DNA, 320 bp, Mus musculus
gaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 504 - protein, 106 aa, Mus musculus
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 505 - DNA, 990 bp, Mus musculus
tccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 506 - protein, 329 aa, Mus musculus
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 507 - protein, 495 aa, Homo sapiens
MPMGSLQPLATLYLLGMLVASCLGRLSWYDPDFQARLTRSNSKCQGQLEVYLKDGWHMVCSQSWGRSSKQWEDPSQASKVCQRLNCGVPLSLGPFLVTYTPQSSIICYGQLGSFSNCSHSRNDMCHSLGLTCLEPQKTTPPTTRPPPTTTPEPTAPPRLQLVAQSGGQHCAGVVEFYSGSLGGTISYEAQDKTQDLENFLCNNLQCGSFLKHLPETEAGRAQDPGEPREHQPLPIQWKIQNSSCTSLEHCFRKIKPQKSGRVLALLCSGFQPKVQSRLVGGSSICEGTVEVRQGAQWAALCDSSSARSSLRWEEVCREQQCGSVNSYRVLDAGDPTSRGLFCPHQKLSQCHELWERNSYCKKVFVTCQDPNPAGLAAGTVASIILALVLLVVLLVVCGPLAYKKLVKKFRQKKQRQWIGPTGMNQNMSFHRNHTATVRSHAENPTASHVDNEYSQPPRNSRLSAYPALEGVLHRSSMQPDNSSDSDYDLHGAQRL

SEQ ID NO: 508 - DNA, 3180 bp, Homo sapiens
acgccaccccgccctctccctctctgagagcgagatacccggccagacaccctcacctgcggtgcccagctgcccaggctgaggcaagagaaggccagaaaccatgcccatggggtctctgcaaccgctggccaccttgtacctgctggggatgctggtcgcttcctgcctcggacggctcagctggtatgacccagatttccaggcaaggctcacccgttccaactcgaagtgccagggccagctggaggtctacctcaaggacggatggcacatggtttgcagccagagctggggccggagctccaagcagtgggaggaccccagtcaagcgtcaaaagtctgccagcggctgaactgtggggtgcccttaagccttggccccttccttgtcacctacacacctcagagctcaatcatctgctacggacaactgggctccttctccaactgcagccacagcagaaatgacatgtgtcactctctgggcctgacctgcttagaaccccagaagacaacacctccaacgacaaggcccccgcccaccacaactccagagcccacagctcctcccaggctgcagctggtggcacagtctggcggccagcactgtgccggcgtggtggagttctacagcggcagcctggggggtaccatcagctatgaggcccaggacaagacccaggacctggagaacttcctctgcaacaacctccagtgtggctccttcttgaagcatctgccagagactgaggcaggcagagcccaagacccaggggagccacgggaacaccagcccttgccaatccaatggaagatccagaactcaagctgtacctccctggagcattgcttcaggaaaatcaagccccagaaaagtggccgagttcttgccctcctttgctcaggtttccagcccaaggtgcagagccgtctggtggggggcagcagcatctgtgaaggcaccgtggaggtgcgccagggggctcagtgggcagccctgtgtgacagctcttcagccaggagctcgctgcggtgggaggaggtgtgccgggagcagcagtgtggcagcgtcaactcctatcgagtgctggacgctggtgacccaacatcccgggggctcttctgtccccatcagaagctgtcccagtgccacgaactttgggagagaaattcctactgcaagaaggtgtttgtcacatgccaggatccaaaccccgcaggcctggccgcaggcacggtggcaagcatcatcctggccctggtgctcctggtggtgctgctggtcgtgtgcggcccccttgcctacaagaagctagtgaagaaattccgccagaagaagcagcgccagtggattggcccaacgggaatgaaccaaaacatgtctttccatcgcaaccacacggcaaccgtccgatcccatgctgagaaccccacagcctcccacgtggataacgaatacagccaacctcccaggaactcccacctgtcagcttatccagctctggaaggggctctgcatcgctcctccatgcagcctgacaactcctccgacagtgactatgatctgcatggggctcagaggctgtaaagaactgggatccatgagcaaaaagccgagagccagacctgtttgtcctgagaaaactgtccgctcttcacttgaaatcatgtccctatttctaccccggccagaacatggacagaggccagaagccttccggacaggcgctgctgccccgagtggcaggccagctcacactctgctgcacaacagctcggccgcccctccacttgtggaagctgtggtgggcagagccccaaaacaagcagccttccaactagagactcgggggtgtctgaagggggccccctttccctgcccgctggggagcggcgtctcagtgaaatcggctttctcctcagactctgtccctggtaaggagtgacaaggaagctcacagctgggcgagtgcattttgaatagttttttgtaagtagtgcttttcctccttcctgacaaatcgagcgctttggcctcttctgtgcagcatccacccctgcggatccctctggggaggacaggaaggggactcccggagacctctgcagccgtggtggtcagaggctgctcacctgagcacaaagacagctctgcacattcaccgcagctgccagccaggggtctgggtgggcaccaccctgacccacagcgtcaccccactccctctgtcttatgactcccctccccaaccccctcatctaaagacaccttcctttccactggctgtcaagcccacagggcaccagtgccacccagggcccggcacaaaggggcgcctagtaaaccttaaccaacttggttttttgcttcacccagcaattaaaagtcccaagctgaggtagtttcagtccatcacagttcatcttctaacccaagagtcagagatggggctggtcatgttcctttggtttgaataactcccttgacgaaaacagactcctctagtacttggagatcttggacgtacacctaatcccatggggcctcggcttccttaactgcaagtgagaagaggaggtctacccaggagcctcgggtctgatcaagggagaggccaggcgcagctcactgcggcggctccctaagaaggtgaagcaacatgggaacacatcctaagacaggtcctttctccacgccatttgatgctgtatctcctgggagcacaggcatcaatggtccaagccgcataataagtctggaagagcaaaagggagttactaggatatggggtgggctgctcccagaatctgctcagctttctgcccccaccaacaccctccaaccaggccttgccttctgagagcccccgtggccaagcccaggtcacagatcttcccccgaccatgctgggaatccagaaacagggaccccatttgtcttcccatatctggtggaggtgagggggctcctcaaaagggaactgagaggctgctcttagggagggcaaaggttcgggggcagccagtgtctcccatcagtgccttttttaataaaagctctttcatctatagtttggccaccatacagtggcctcaaagcaaccatggcctacttaaaaaccaaaccaaaaataaagagtttagttgaggagaaaaaaaaaaaaaaaaaaaaaaaaa

(19) United States
(12) Patent Application Publication
(10) Pub. No.: US 2011/0250203 A1
(43) Pub. Date: Oct. 13, 2011

(54) ANTI-CD5 ANTIBODIES

(75) Inventors: Josephine L. K. Klitgaard, Kgs. Lyngby (DK); Charles Pyke, Hilleroed (DK); Mikke Wandahl Pedersen, Alleroed (DK); Klaus Koefoed, Kobenhavn S (DK)

(73) Assignee: Symphogen A/S, Kgs. Lyngby (DK)

(21) Appl. No.: 13/061,420

(22) PCT Filed: Aug. 28, 2009
(86) PCT No.: PCT/DK09/50218

§ 371 Date: May 16, 2011

Related U.S. Application Data
(60) Provisional application No. 61/093,700, filed on Sep. 2, 2008.

(30) Foreign Application Priority Data
Aug. 29, 2008 (DK) PA 2008 01191

(51) Int. Cl. A61K 39/395; C12P 21/00; A61P 35/00; C40B 40/02; A61P 19/02; A61P 35/02; C07K 16/28; C12N 5/071

(52) U.S. Cl. 424/136.1; 424/173.1; 424/133.1; 530/387.3; 435/69.6; 435/375; 506/14

(57) ABSTRACT

The present invention relates to the field of compositions comprising anti-CD5 antibodies. In particular, the present invention concerns an antibody composition comprising at least two anti-CD5 antibodies capable of binding distinct CD5 epitopes. The invention further concerns bi-specific molecules having the binding specificities of said antibody compositions. The invention also relates topharmaceutical compositions, use of antibody compositions and methods for manufacturing antibody compositions. The invention further relates to cell banks and a method for killing cells.

### FIELD OF INVENTION

The present invention relates to the field of compositions comprising anti-CD5 antibodies.

### BACKGROUND OF INVENTION

Antibodies are molecules produced by the immune system when challenged with foreign invading pathogens such as bacteria and viruses. The antibody molecules consist of two heavy chains (HC) and two light chains (LC), connected by disulphide bridges to form a V-shaped molecule with the variable binding domain present on the tip of each arm. The molecules are characterized by high variability and very strong binding to foreign matter (typically proteins), so-called antigens. Antibodies exert their function by binding to specific epitopes on the antigens. Once bound, different effector functions can be mediated through the constant part of the antibody, the Fc region. Dependent on the antibody isotype, effector functions such as complement lysis, cellular killing, phagocytosis, etc., can be accomplished. Apart from the antibody structure, different aspects of the antibody biology influence the effect of naturally occurring endogenous antibodies and treatment with antibody-based drugs. These aspects include affinity of the antibodies, as well as the reaction rate (i.e. how fast the antibodies bind to the epitope), the location on the antigen of the bound epitopes, the number of antibodies targeting different epitopes represented in the composition and weather or not they bind immunogenic epitopes.

### Use of Antibodies as Therapeutic Drugs

Polyclonal antibody preparations derived from blood plasma—the so-called hyperimmune immunoglobulin products—have traditionally been used with success for treatment of diseases characterized by targets of high complexity, such as infections with cytomegalovirus or Hepatitis B virus. However, blood-derived products have a number of inherent disadvantages, including supply shortage, high batch-to-batch variation as well as safety risks associated with potential transfer of infectious agents from the blood to the patient. During the past 10-15 years, much focus has been put into investigating the therapeutic potential of recombinant antibodies, and this focus has turned out to be a highly rewarding investment. At present, more than 20% of the drugs in clinical development globally are antibody-derived, amounting to a total of about 400 potential drugs on the future world market. The ˜20 recombinant antibody therapeutics presently approved for marketing are all monoclonal antibodies. Technologies for generation and industrial production of recombinant polyclonal antibodies have so far been lacking. However, the advantages of and request for polyclonal antibody therapeutics, targeting more than a single antigen-epitope, have already been observed. The aim is to increase the quality of antibody-based drugs by re-introducing the concept of polyclonality previously documented by the use of hyper immune immunoglobulin products in future recombinant antibody-based drugs.

### Chronic Lymphocytic Leukaemia and CD5

CLL is the most common form of leukaemia in the Western world. CLL cells express CD19 and CD20 on the cell membrane along with CD5 and CD23. Thus, this phenotype can be distinguished from the other CD5 positive B-cell disease Mantle Cell Lymphoma (MCL), which lacks expression of CD23, but express another surface molecule called FMC7. Both diseases are incurable with conventional chemotherapy. At the present time Fludarabine based regimens represent the most effective therapy for controlling CLL. The standard dose regimen using the anti-CD20 antibody Rituximab as mono-therapy, has very limited effect in CLL, compared to the results in other indolent B-cell lymphomas/leukaemia. The only antibody with proven single agent efficacy in CLL is the anti-CD52 antibody Campath-1H, registered for chemo-refractory CLL. However, the profound immunodeficiency associated with Campath-1H limits its general application in CLL. Current strategies for improving the results in CLL focus on antibody chemotherapy combinations and the development of antibodies targeting other antigens than CD20 and CD52, e.g. CD23, CD40, CD40-ligand and HLA-DR. We suggest that the unexploited CD5 antigen, which is characteristic for CLL cells, is an attractive target for antibody based passive immunotherapy in CLL.

CD5 is a type I glycoprotein and a member of the scavenger-receptor family. CD5 is expressed by thymocytes, mature T cells and a subset of mature B cells and has been shown to be involved in modulation of lymphocyte activation and in the differentiation process. CD72, gp80-40 and Ig framework structures are purposed ligands for CD5 and their interaction with CD5 have been shown in mice, the exact role and structural characteristics of these interactions remain to be clarified. CD5 is associated with CD79a and CD79b transduction partner of surface IgM in the vicinity of the B-cell receptor (BCR) and CD5 signalling is mediated by co-precipitation with the BCR and CD79a and CD79b into lipid rafts. CD79a and CD79b are phosphorylated by the Lyn and other tyrosine kinases such as Syk, and Zap70 as well as the tyrosine phosphatase SHP-1 have been reported to be mediators of this signal transduction also. Truncated forms of CD79b have been observed in CLL cells, therefore is has been suggested that impaired intra cellular signalling might be important for survival of CLL cells. Cross-linking of CD5 with a monoclonal anti-CD5 antibody can induce apoptosis of CLL cells, however the induction of this signal did not happen unless CD5 was translocated into lipid rafts with BCR and BCR-associated molecules. As the location of an epitope is crucial for the ability of an antibody to effect signals delivered through a receptor, we believe that a range of antibodies with many specificities towards CD5 will unveil knowledge about the signaling role of CD5 in CLL cells and in normal cells and that this knowledge will contribute to the development of an effective antibody therapy against CLL.

### Polyclonal Antibodies Against CD5

As a polyclonal antibody composition contains several antibody-specificities and thereby targets several epitopes, we argue that a more effective blocking of the signal pathway activated by CD72 and other potential ligand for CD5 could be achieved with a polyclonal antibody composition as compared to a mAb. Targeting of specific epitopes can be crucial for the ability of the mAb to affect signals delivered through a receptor. As the specificity of anti-CD20 antibody (Rituximab) has been shown to directly influence the type of effector function induced in vivo and as antibodies against Her-2 with different epitope-specificities have been shown to induce different anti-tumor activities, we speculate that a polyclonal antibody composition containing antibodies of multiple specificities will be more efficient in mediating killing of tumor cells. In addition, it is likely that the high density of antibodies created on the surface by a polyclonal antibody composition targeting multiple antigen-epitopes, will increase the activation of effector function such as complement-mediated lysis and ADCC, which similar to apoptosis have been shown to play a major role in the treatment of CLL. It is thus very likely that an overall more effective therapy can be accomplished with a polyclonal antibody composition.

As mentioned above, the monoclonal anti-CD52 antibody, Campath-1H, has been shown to be efficient against CLL in heavily pre-treated patients, but treatment is accompanied by significant immunosuppression and occurrence of opportunistic infections. This is because CD52 is expressed on all leucocytes except for plasma cells, and Campath-1 H thus targets both CLL and healthy cells. CD5 on the other hand is not expressed on NK cells and healthy B cells and an anti-CLL antibody therapeutic targeting CD5 will therefore preferentially target cancer cells, which will be beneficial for the patients, as healthy cells are eliminated less often and the patients should experience minimal immunosuppression.

T101 is a monoclonal mouse IgG2a against CD5, developed for treatment of patients suffering from Cutaneous T-Cell Lymphoma (CTCL) and Rheumatoid Arthritis. Due to a lack of therapeutic effect in phase II studies, clinical development T101 has been halted. It is not known why T101 did not have clinical effect, and it is possible that it relates to the fact that it is a fully murine antibody that inevitably will lead to a neutralizing HAMA (Human Anti-Mouse Antibody) response. Nevertheless, it is conceivable that targeting several CD5 epitopes with a polyclonal antibody composition could lead to a significant increase in inhibition of CD5 functions as compared to a monoclonal antibody such as T101, e.g. by increasing receptor internalization, thus leading to clinical anti-tumor effects. In addition, as we intend to produce CD5-specific chimeric antibodies containing mouse variable regions and human constant regions, the major part of the HAMA response observed with the fully murine T101 antibody will not be seen. If antibodies against the variable region of the chimeric antibodies should occur, our previous studies with neutralizing antibodies against monoclonal and polyclonal antibodies ex vivo have indicated that a polyclonal antibody composition is less susceptible to neutralizing antibodies than monoclonal antibodies. Therefore, a polyclonal anti-CD5 antibody composition will most likely remain pharmacologically active if induction of neutralizing antibodies should occur.

### Effector Mechanisms

The effect on a cellular level of an antibody binding an antigen on the surface of a cell varies depending on the specific antibody bound. Important effector mechanisms include Antibody Dependent Cellular Cytotoxicity (ADCC) and Complement Dependent Cytotoxicity (CDC). The ADCC effector mechanism is characterized by effector cells of the immune system actively lysing a target cell that has been bound by specific antibodies. It is one of the mechanisms through which antibodies, as part of the humoral immune response, can act to limit and contain infection. Classical ADCC is mediated by natural killer (NK) cells. However, monocytes and polymorphonuclear granulocytes (PMN's) can also mediate ADCC. ADCC is part of the adaptive immune response due to its dependence on a prior antibody response. The typical ADCC involves activation of NK cells and is dependent on the recognition of antibody-coated infected cells by Fc receptors on the surface of the NK cell. The Fc receptors recognize the Fc (crystalline) portion of antibodies such as IgG, which bind to the surface of a pathogen-infected target cell. The most common Fc receptor that exists on the surface of NK Cell is called CD16 or FcγRIII. Once bound to the Fc receptor of IgG the Natural Killer cell releases cytokines such as IFN-γ, and cytotoxic granules containing perforin and granzymes that enter the target cell and promote cell death by triggering apoptosis. This is similar to, but independent of, responses by cytotoxic T cells (CTLs). The level of ADCC is dependent on several factors including IgG subtype (IgM>IgG1>IgG2), antibody density on target cells, antibody glycosylation pattern as well as the properties of the target itself.

CDC is an alternative effector mechanism by which antibody binding to cellular antigens can lead to neutralization of the bound cells. Antibodies are capable of activating the so-called classical complement pathway. In the classical complement pathway, the bound antibody recruits the proteins of the complement system, which through a series of interactions lead to killing of the bound cell. The complement system consists of a number of small proteins found in the blood, normally circulating as inactive zymogens. When stimulated by one of several triggers, proteases in the system cleave specific proteins to release cytokines and initiate an amplifying cascade of further cleavages. The end result of this activation cascade is massive amplification of the response and activation of the cell-killing membrane attack complex. Over 20 proteins and protein fragments make up the complement system, including serum proteins, serosal proteins, and cell membrane receptors.

Different studies comprising therapeutic antibodies against cancer antigens show that anti-tumor activity requires activation of effector mechanisms such as ADCC and CDC which are activated through binding to the antibody Fc region. Therefore, another issue concerning the effect of antibody therapeutics is the interaction of the antibody Fc region and the recruited effector molecules. The binding of the Fc region of IgG antibodies to the Fc-receptors on effector molecules such as macrophages, NK cells and complement proteins, is influenced by the glycosylation of the antibody CH2 domain. Especially the degree of fucose on the N-linked oligosaccharide at asparagine 297 has been shown to influence the binding of the IgG Fc region to the FcγIII receptor (CD16) on NK cells. The effect of glycosylation in regard to complement activation remains to be elucidated. The antibody glycosylation is species-specific and thus the nature of the production cell line has major impact on the antibody's ability to bind and mediate effector functions. Due to the above-described differences in antibody-glycosylation, we argue that antibodies expressed in CHO cells and in the human Per.C6 cells will influence the therapeutic effect of antibody-based drugs differently.

### SUMMARY OF INVENTION

In one aspect, the present invention relates to antibody composition comprising at least two anti-CD5 antibodies binding distinct CD5 epitopes.

In a further aspect of the invention, the said composition comprises an anti-CD5 antibody molecule selected from the group consisting of any one of the antibodies 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and 37 as indicated in table 1.

Preferably, said composition comprises an antibody comprising the VL and VH sequences of any one of the antibodies 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and 37 as indicated in table 1. The VL and VH sequences of each antibody (listed by name) are provided in Table 2 and 3.

Preferably said composition comprises an antibody comprising the CDRH1, CDRH2, CDRH3, CDRL1, CDRL2, and CDRL3 sequences of any one of the antibodies 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and 37 as indicated in table 1.

Preferably said composition comprises an antibody binding to the same epitope as any one of the antibodies 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and 37 as indicated in table 1.

Preferably said composition comprises an antibody capable of inhibiting the binding to human CD5 of any one of the antibodies 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and 37 as indicated in table 1.

The following are embodiments of the invention:

Antibody composition according to the invention, wherein said antibody composition comprises an anti-CD5 antibody molecule selected from the group consisting of antibody 1, an antibody comprising the VL and VH sequences of antibody 1, an antibody comprising the CDRH1, CDRH2, CDRH3, CDRL1, CDRL2, and CDRL3 sequences of antibody 1, an antibody binding to the same epitope as antibody 1, and an antibody capable of inhibiting the binding of antibody 1 to human CD5.

Antibody composition according to the invention, wherein said antibody composition comprises an anti-CD5 antibody molecule selected from the group consisting of antibody 2, an antibody comprising the VL and VH sequences of antibody 2, an antibody comprising the CDRH1, CDRH2, CDRH3, CDRL1, CDRL2, and CDRL3 sequences of antibody 2, an antibody binding to the same epitope as antibody 2, and an antibody capable of inhibiting the binding of antibody 2 to human CD5.

Antibody composition according to the invention, wherein said antibody composition comprises an anti-CD5 antibody molecule selected from the group consisting of antibody 3, an antibody comprising the VL and VH sequences of antibody 3, an antibody comprising the CDRH1, CDRH2, CDRH3, CDRL1, CDRL2, and CDRL3 sequences of antibody 3, an antibody binding to the same epitope as antibody 3, and an antibody capable of inhibiting the binding of antibody 3 to human CD5.

Antibody composition according to the invention, wherein said antibody composition comprises an anti-CD5 antibody molecule selected from the group consisting of antibody 4, an antibody comprising the VL and VH sequences of antibody 4, an antibody comprising the CDRH1, CDRH2, CDRH3, CDRL1, CDRL2, and CDRL3 sequences of antibody 4, an antibody binding to the same epitope as antibody 4, and an antibody capable of inhibiting the binding of antibody 4 to human CD5.

Antibody composition according to the invention, wherein said antibody composition comprises an anti-CD5 antibody molecule selected from the group consisting of antibody 5, an antibody comprising the VL and VH sequences of antibody 5, an antibody comprising the CDRH1, CDRH2, CDRH3, CDRL1, CDRL2, and CDRL3 sequences of antibody 5, an antibody binding to the same epitope as antibody 5, and an antibody capable of inhibiting the binding of antibody 5 to human CD5.

Antibody composition according to the invention, wherein said antibody composition comprises an anti-CD5 antibody molecule selected from the group consisting of antibody 6, an antibody comprising the VL and VH sequences of antibody 6, an antibody comprising the CDRH1, CDRH2, CDRH3, CDRL1, CDRL2, and CDRL3 sequences of antibody 6, an antibody binding to the same epitope as antibody 6, and an antibody capable of inhibiting the binding of antibody 6 to human CD5.

Antibody composition according to the invention, wherein said antibody composition comprises an anti-CD5 antibody molecule selected from the group consisting of antibody 7, an antibody comprising the VL and VH sequences of antibody 7, an antibody comprising the CDRH1, CDRH2, CDRH3, CDRL1, CDRL2, and CDRL3 sequences of antibody 7, an antibody binding to the same epitope as antibody 7, and an antibody capable of inhibiting the binding of antibody 7 to human CD5.

Antibody composition according to the invention, wherein said antibody composition comprises an anti-CD5 antibody molecule selected from the group consisting of antibody 8, an antibody comprising the VL and VH sequences of antibody 8, an antibody comprising the CDRH1, CDRH2, CDRH3, CDRL1, CDRL2, and CDRL3 sequences of antibody 8, an antibody binding to the same epitope as antibody 8, and an antibody capable of inhibiting the binding of antibody 8 to human CD5.

Antibody composition according to the invention, wherein said antibody composition comprises an anti-CD5 antibody molecule selected from the group consisting of antibody 9, an antibody comprising the VL and VH sequences of antibody 9, an antibody comprising the CDRH1, CDRH2, CDRH3, CDRL1, CDRL2, and CDRL3 sequences of antibody 9, an antibody binding to the same epitope as antibody 9, and an antibody capable of inhibiting the binding of antibody 9 to human CD5.

Antibody composition according to the invention, wherein said antibody composition comprises an anti-CD5 antibody molecule selected from the group consisting of antibody 10, an antibody comprising the VL and VH sequences of antibody 10, an antibody comprising the CDRH1, CDRH2, CDRH3, CDRL1, CDRL2, and CDRL3 sequences of antibody 10, an antibody binding to the same epitope as antibody 10, and an antibody capable of inhibiting the binding of antibody 10 to human CD5.

Antibody composition according to the invention, wherein said antibody composition comprises an anti-CD5 antibody molecule selected from the group consisting of antibody 11, an antibody comprising the VL and VH sequences of antibody 11, an antibody comprising the CDRH1, CDRH2, CDRH3, CDRL1, CDRL2, and CDRL3 sequences of antibody 11, an antibody binding to the same epitope as antibody 11, and an antibody capable of inhibiting the binding of antibody 11 to human CD5.

Antibody composition according to the invention, wherein said antibody composition comprises an anti-CD5 antibody molecule selected from the group consisting of antibody 12, an antibody comprising the VL and VH sequences of antibody 12, an antibody comprising the CDRH1, CDRH2, CDRH3, CDRL1, CDRL2, and CDRL3 sequences of antibody 12, an antibody binding to the same epitope as antibody 12, and an antibody capable of inhibiting the binding of antibody 12 to human CD5.

Antibody composition according to the invention, wherein said antibody composition comprises an anti-CD5 antibody molecule selected from the group consisting of antibody 13, an antibody comprising the VL and VH sequences of antibody 13, an antibody comprising the CDRH1, CDRH2, CDRH3, CDRL1, CDRL2, and CDRL3 sequences of antibody 13, an antibody binding to the same epitope as antibody 13, and an antibody capable of inhibiting the binding of antibody 13 to human CD5.

Antibody composition according to the invention, wherein said antibody composition comprises an anti-CD5 antibody molecule selected from the group consisting of antibody 14, an antibody comprising the VL and VH sequences of antibody 14, an antibody comprising the CDRH1, CDRH2, CDRH3, CDRL1, CDRL2, and CDRL3 sequences of antibody 14, an antibody binding to the same epitope as antibody 14, and an antibody capable of inhibiting the binding of antibody 14 to human CD5.

Antibody composition according to the invention, wherein said antibody composition comprises an anti-CD5 antibody molecule selected from the group consisting of antibody 15, an antibody comprising the VL and VH sequences of antibody 15, an antibody comprising the CDRH1, CDRH2, CDRH3, CDRL1, CDRL2, and CDRL3 sequences of antibody 15, an antibody binding to the same epitope as antibody 15, and an antibody capable of inhibiting the binding of antibody 15 to human CD5.

Antibody composition according to the invention, wherein said antibody composition comprises an anti-CD5 antibody molecule selected from the group consisting of antibody 16, an antibody comprising the VL and VH sequences of antibody 16, an antibody comprising the CDRH1, CDRH2, CDRH3, CDRL1, CDRL2, and CDRL3 sequences of antibody 16, an antibody binding to the same epitope as antibody 16, and an antibody capable of inhibiting the binding of antibody 16 to human CD5.

Antibody composition according to the invention, wherein said antibody composition comprises an anti-CD5 antibody molecule selected from the group consisting of antibody 17, an antibody comprising the VL and VH sequences of antibody 17, an antibody comprising the CDRH1, CDRH2, CDRH3, CDRL1, CDRL2, and CDRL3 sequences of antibody 17, an antibody binding to the same epitope as antibody 17, and an antibody capable of inhibiting the binding of antibody 17 to human CD5.

Antibody composition according to the invention, wherein said antibody composition comprises an anti-CD5 antibody molecule selected from the group consisting of antibody 18, an antibody comprising the VL and VH sequences of antibody 18, an antibody comprising the CDRH1, CDRH2, CDRH3, CDRL1, CDRL2, and CDRL3 sequences of antibody 18, an antibody binding to the same epitope as antibody 18, and an antibody capable of inhibiting the binding of antibody 18 to human CD5.

Antibody composition according to the invention, wherein said antibody composition comprises an anti-CD5 antibody molecule selected from the group consisting of antibody 19, an antibody comprising the VL and VH sequences of antibody 19, an antibody comprising the CDRH1, CDRH2, CDRH3, CDRL1, CDRL2, and CDRL3 sequences of antibody 19, an antibody binding to the same epitope as antibody 19, and an antibody capable of inhibiting the binding of antibody 19 to human CD5.

Antibody composition according to the invention, wherein said antibody composition comprises an anti-CD5 antibody molecule selected from the group consisting of antibody 20, an antibody comprising the VL and VH sequences of antibody 20, an antibody comprising the CDRH1, CDRH2, CDRH3, CDRL1, CDRL2, and CDRL3 sequences of antibody 20, an antibody binding to the same epitope as antibody 20, and an antibody capable of inhibiting the binding of antibody 20 to human CD5.

Antibody composition according to the invention, wherein said antibody composition comprises an anti-CD5 antibody molecule selected from the group consisting of antibody 21, an antibody comprising the VL and VH sequences of antibody 21, an antibody comprising the CDRH1, CDRH2, CDRH3, CDRL1, CDRL2, and CDRL3 sequences of antibody 21, an antibody binding to the same epitope as antibody 21, and an antibody capable of inhibiting the binding of antibody 21 to human CD5.

Antibody composition according to the invention, wherein said antibody composition comprises an anti-CD5 antibody molecule selected from the group consisting of antibody 22, an antibody comprising the VL and VH sequences of antibody 22, an antibody comprising the CDRH1, CDRH2, CDRH3, CDRL1, CDRL2, and CDRL3 sequences of antibody 22, an antibody binding to the same epitope as antibody 22, and an antibody capable of inhibiting the binding of antibody 22 to human CD5.

Antibody composition according to the invention, wherein said antibody composition comprises an anti-CD5 antibody molecule selected from the group consisting of antibody 23, an antibody comprising the VL and VH sequences of antibody 23, an antibody comprising the CDRH1, CDRH2, CDRH3, CDRL1, CDRL2, and CDRL3 sequences of antibody 23, an antibody binding to the same epitope as antibody 23, and an antibody capable of inhibiting the binding of antibody 23 to human CD5.

Antibody composition according to the invention, wherein said antibody composition comprises an anti-CD5 antibody molecule selected from the group consisting of antibody 24, an antibody comprising the VL and VH sequences of antibody 24, an antibody comprising the CDRH1, CDRH2, CDRH3, CDRL1, CDRL2, and CDRL3 sequences of antibody 24, an antibody binding to the same epitope as antibody 24, and an antibody capable of inhibiting the binding of antibody 24 to human CD5.

Antibody composition according to the invention, wherein said antibody composition comprises an anti-CD5 antibody molecule selected from the group consisting of antibody 25, an antibody comprising the VL and VH sequences of antibody 25, an antibody comprising the CDRH1, CDRH2, CDRH3, CDRL1, CDRL2, and CDRL3 sequences of antibody 25, an antibody binding to the same epitope as antibody 25, and an antibody capable of inhibiting the binding of antibody 25 to human CD5.

Antibody composition according to the invention, wherein said antibody composition comprises an anti-CD5 antibody molecule selected from the group consisting of antibody 26, an antibody comprising the VL and VH sequences of antibody 26, an antibody comprising the CDRH1, CDRH2, CDRH3, CDRL1, CDRL2, and CDRL3 sequences of antibody 26, an antibody binding to the same epitope as antibody 26, and an antibody capable of inhibiting the binding of antibody 26 to human CD5.

Antibody composition according to the invention, wherein said antibody composition comprises an anti-CD5 antibody molecule selected from the group consisting of antibody 27, an antibody comprising the VL and VH sequences of antibody 27, an antibody comprising the CDRH1, CDRH2, CDRH3, CDRL1, CDRL2, and CDRL3 sequences of antibody 27, an antibody binding to the same epitope as antibody 27, and an antibody capable of inhibiting the binding of antibody 27 to human CD5.

Antibody composition according to the invention, wherein said antibody composition comprises an anti-CD5 antibody molecule selected from the group consisting of antibody 28, an antibody comprising the VL and VH sequences of antibody 28, an antibody comprising the CDRH1, CDRH2, CDRH3, CDRL1, CDRL2, and CDRL3 sequences of antibody 28, an antibody binding to the same epitope as antibody 28, and an antibody capable of inhibiting the binding of antibody 28 to human CD5.

Antibody composition according to the invention, wherein said antibody composition comprises an anti-CD5 antibody molecule selected from the group consisting of antibody 29, an antibody comprising the VL and VH sequences of antibody 29, an antibody comprising the CDRH1, CDRH2, CDRH3, CDRL1, CDRL2, and CDRL3 sequences of antibody 29, an antibody binding to the same epitope as antibody 29, and an antibody capable of inhibiting the binding of antibody 29 to human CD5.

Antibody composition according to the invention, wherein said antibody composition comprises an anti-CD5 antibody molecule selected from the group consisting of antibody 30, an antibody comprising the VL and VH sequences of antibody 30, an antibody comprising the CDRH1, CDRH2, CDRH3, CDRL1, CDRL2, and CDRL3 sequences of antibody 30, an antibody binding to the same epitope as antibody 30, and an antibody capable of inhibiting the binding of antibody 30 to human CD5.

Antibody composition according to the invention, wherein said antibody composition comprises an anti-CD5 antibody molecule selected from the group consisting of antibody 31, an antibody comprising the VL and VH sequences of antibody 31, an antibody comprising the CDRH1, CDRH2, CDRH3, CDRL1, CDRL2, and CDRL3 sequences of antibody 31, an antibody binding to the same epitope as antibody 31, and an antibody capable of inhibiting the binding of antibody 31 to human CD5.

Antibody composition according to the invention, wherein said antibody composition comprises an anti-CD5 antibody molecule selected from the group consisting of antibody 32, an antibody comprising the VL and VH sequences of antibody 32, an antibody comprising the CDRH1, CDRH2, CDRH3, CDRL1, CDRL2, and CDRL3 sequences of antibody 32, an antibody binding to the same epitope as antibody 32, and an antibody capable of inhibiting the binding of antibody 32 to human CD5.

Antibody composition according to the invention, wherein said antibody composition comprises an anti-CD5 antibody molecule selected from the group consisting of antibody 33, an antibody comprising the VL and VH sequences of antibody 33, an antibody comprising the CDRH1, CDRH2, CDRH3, CDRL1, CDRL2, and CDRL3 sequences of antibody 33, an antibody binding to the same epitope as antibody 33, and an antibody capable of inhibiting the binding of antibody 33 to human CD5.

Antibody composition according to the invention, wherein said antibody composition comprises an anti-CD5 antibody molecule selected from the group consisting of antibody 34, an antibody comprising the VL and VH sequences of antibody 34, an antibody comprising the CDRH1, CDRH2, CDRH3, CDRL1, CDRL2, and CDRL3 sequences of antibody 34, an antibody binding to the same epitope as antibody 34, and an antibody capable of inhibiting the binding of antibody 34 to human CD5.

Antibody composition according to the invention, wherein said antibody composition comprises an anti-CD5 antibody molecule selected from the group consisting of antibody 35, an antibody comprising the VL and VH sequences of antibody 35, an antibody comprising the CDRH1, CDRH2, CDRH3, CDRL1, CDRL2, and CDRL3 sequences of antibody 35, an antibody binding to the same epitope as antibody 35, and an antibody capable of inhibiting the binding of antibody 35 to human CD5.

Antibody composition according to the invention, wherein said antibody composition comprises an anti-CD5 antibody molecule selected from the group consisting of antibody 36, an antibody comprising the VL and VH sequences of antibody 36, an antibody comprising the CDRH1, CDRH2, CDRH3, CDRL1, CDRL2, and CDRL3 sequences of antibody 36, an antibody binding to the same epitope as antibody 36, and an antibody capable of inhibiting the binding of antibody 36 to human CD5.

Antibody composition according to the invention, wherein said antibody composition comprises an anti-CD5 antibody molecule selected from the group consisting of antibody 37, an antibody comprising the VL and VH sequences of antibody 37, an antibody comprising the CDRH1, CDRH2, CDRH3, CDRL1, CDRL2, and CDRL3 sequences of antibody 37, an antibody binding to the same epitope as antibody 37, and an antibody capable of inhibiting the binding of antibody 37 to human CD5.

In another aspect, the invention relates to a bi-specific binding molecule having the binding specificities of any one of the antibody compositions according to the invention.

In a further aspect, the invention relates to a pharmaceutical composition comprising as an active ingredient an antibody composition according to the invention or a bi-specific binding molecule according to the invention.

In another aspect, the invention relates to an antibody composition according to the invention or a bi-specific binding molecule according to the invention for use as a medicament.

In a further aspect, the invention relates to the use of an antibody composition according to the invention or a bi-specific binding molecule according to the invention in the manufacture of a medicament.

In another aspect, the invention relates to a method of treatment comprising administering to a patient in need thereof a pharmaceutical composition according to the invention.

In a further aspect, the invention relates to a method for manufacturing an antibody composition, said method comprising the steps of:
transfecting a first population of eukaryotic cells with a first expression construct coding for a first antibody comprising a first cognate pair of VH and VL chains capable of binding a first distinct CD5 epitope;
transfecting a second population of eukaryotic cells with a second expression construct coding for a second antibody comprising a second cognate pair of VH and VL chains capable of binding a second distinct CD5 epitope;
optionally repeating step b) for third or further populations, expression constructs, cognate pairs, and CD5 epitopes;
selecting transfected first, second and optionally further cell populations;
combining the transfected populations in one pot to obtain a cell bank;
culturing cells from the cell bank under conditions allowing expression of the antibodies; and
recovering and purifying the antibody composition from the supernatant.

In another aspect, the invention relates to a cell bank comprising at least two sub-populations of eukaryotic cells, wherein each sub-population is transfected or transduced with one expression construct coding for an antibody comprising a cognate pair of VH and VL chains capable of binding a distinct CD5 epitope.

In a further aspect, the invention relates to a method of killing cells expressing CD5 comprising administering to cells expressing CD5 an antibody composition according to the invention or a bi-specific binding molecule according to the invention, and thereby killing the CD5 expressing cells.

Preferred embodiments of the invention are set out in the dependent claims.

### Definitions

Antibody: The term “antibody” describes a functional component of serum and is often referred to either as a collection of molecules (antibodies or immunoglobulin) or as one molecule (the antibody molecule or immunoglobulin molecule). An antibody molecule is capable of binding to or reacting with a specific antigenic determinant (the antigen or the antigenic epitope), which in turn may lead to induction of immunological effector mechanisms. An individual antibody molecule is usually regarded as monospecific, and a composition of antibody molecules may be monoclonal (i.e., consisting of identical antibody molecules) or polyclonal (i.e., consisting of two or more different antibody molecules reacting with the same or different epitopes on the same antigen or even on distinct, different antigens). Each antibody molecule has a unique structure that enables it to bind specifically to its corresponding antigen, and all natural antibody molecules have the same overall basic structure of two identical light chains and two identical heavy chains. Antibodies are also known collectively as immunoglobulins. The terms antibody or antibodies as used herein are also intended to include chimeric and single chain antibodies, as well as binding fragments of antibodies, such as Fab, Fv fragments or scFv fragments, as well as multimeric forms such as dimeric IgA molecules or pentavalent IgM. An antibody may be human, murine, chimeric, humanised, or reshaped.

CDR: The term “CDR”—complementarity determining region is as defined in Lefranc et al (2003) IMGT unique numbering for immunoglobulin and T cell receptor variable domains and Ig superfamily V-like domains. Dev. Comp Immunol 27, 55-77.

The terms “a distinct member of a recombinant polyclonal protein” denotes one protein molecule of a protein composition comprising different, but homologous protein molecules, where each protein molecule is homologous to the other molecules of the composition, but also contains one or more stretches of variable polypeptide sequence, which is/are characterized by differences in the amino acid sequence between the individual members of the polyclonal protein.

Cognate VH and VL coding pair : The term “cognate VH and VL coding pair” describes an original pair of VH and VL coding sequences contained within or derived from the same antibody producing cell. Thus, a cognate VH and VL pair represents the VH and VL pairing originally present in the donor from which such a cell is derived. The term “an antibody expressed from a VH and VL coding pair” indicates that an antibody or an antibody fragment is produced from a vector, plasmid or similar containing the VH and VL coding sequence. When a cognate VH and VL coding pair is expressed, either as a complete antibody or as a stable fragment thereof, they preserve the binding affinity and specificity of the antibody originally expressed from the cell they are derived from. A library of cognate pairs is also termed a repertoire or collection of cognate pairs, and may be kept individually or pooled.

Distinct epitopes: The term “distinct epitopes” means that the amino acid sequences constituting the epitopes are different. Distinct epitopes can be overlapping epitopes, in that two distinct epitopes may share part of their amino acid sequence.

Epitope: The term “epitope” is used to describe a proportion of a larger molecule or a part of a larger molecule (e.g. antigen or antigenic site) having antigenic or immunogenic activity in an animal, preferably a mammal, and most preferably in a human. An epitope having immunogenic activity is a portion of a larger molecule that elicits an antibody response in an animal. An epitope having antigenic activity is a portion of a larger molecule to which an antibody immunospecifically binds as determined by any method well known in the art, for example, by the immunoassays described herein. Antigenic epitopes need not necessarily be immunogenic. An antigen is a substance to which an antibody or antibody fragment immunospecifically binds, e.g. toxin, virus, bacteria, proteins or DNA. An antigen or antigenic site often has more than one epitope, unless they are very small, and is often capable of stimulating an immune response. Epitopes may be linear or conformational. A linear epitope consists of about 6 to 10 adjacent amino acids on a protein molecule that is recognized by an antibody. In contrast, conformational epitope consists of amino acids that are not arranged sequentially. Here the antibody recognizes only the 3-dimensional structure. When a protein molecule folds into a three dimensional structure the amino acids forming the epitope are juxtaposed enabling the antibody to recognize the sequence. In a denatured protein only the linear epitope may be recognized. A conformational epitope, by definition, must be on the outside of the folded protein. An antibody that recognizes the conformational epitope may only bind under mild, non-denaturing procedures. Antibodies binding to different epitopes on the same antigen can have varying effects on the activity of the antigen they bind depending on the location of the epitope. An antibody binding to an epitope in an active site of the antigen may block the function of the antigen completely, whereas another antibody binding at a different epitope may have no or little effect on the activity of the antigen alone. Such antibodies may however still activate complement and thereby result in the elimination of the antigen, and may result in synergistic effects when combined with one or more antibodies binding at different epitopes on the same antigen. In the present invention, the epitope is preferably a proportion of the extracellular domain of CD5. Antigens of the present invention are preferably extracellular domain CD5 proteins, polypeptides or fragments thereof to which an antibody or antibody fragment immunospecifically binds. A CD5 associated antigen may also be an analogue or derivative of the extracellular domain of CD5 polypeptide or fragment thereof to which an antibody or antibody fragment immunospecifically binds. Antibodies capable of competing with each other for binding to the same antigen may bind the same or overlapping epitopes or may have a binding site in the close vicinity of one another, so that competition is mainly caused by steric hindrance.

Immunoglobulin: The term “immunoglobulin” commonly is used as a collective designation of the mixture of antibodies found in blood or serum, but may also be used to designate a mixture of antibodies derived from other sources.

Immunoglobulin molecule: The term “immunoglobulin molecule” denotes an individual antibody molecule, e.g., as being a part of immunoglobulin, or part of any polyclonal or monoclonal antibody composition.

Overlapping epitopes: As used herein, the term “overlapping epitopes” means that the amino acid sequences of the epitopes overlap, i.e. that the epitopes share at least one amino acid residue, which is present in both epitopes. Antibodies binding overlapping epitopes inhibit the binding to the antigen of each other. For instance, binding of a first antibody to a first epitope overlapping with a second epitope, where the second epitope is already bound by a second antibody, may be inhibited by at least 10%, such as by at least 20%, for example at least 30%, such as by at least 40%, for example at least 50%, such as by at least 60%, for example at least 70%, such as by at least 80%, for example at least 90%, such as 100%. An analysis for “overlapping epitopes” of antibody pairs is typically determined by binding experiments under saturating antibody conditions with either FACS analysis on cells expressing CD5 and individually fluorescent labelled antibodies, or Surface Plasmon Resonance using CD5 antigen captured or conjugated to a flow cell surface as described in the examples.

Polyclonal antibody: The term “polyclonal antibody” describes a composition of different antibody molecules which is capable of binding to or reacting with several different specific antigenic determinants on the same or on different antigens. Usually, the variability of a polyclonal antibody is thought to be located in the so-called variable regions of the polyclonal antibody. However, in the context of the present invention, polyclonality can also be understood to describe differences between the individual antibody molecules residing in so-called constant regions, e.g., as in the case of mixtures of antibodies containing two or more antibody isotypes such as the human isotypes IgG1, IgG2, IgG3, IgG4, IgA1, and IgA2, or the murine isotypes IgG1, IgG2a, IgG2b, IgG3, and IgA. For purposes of the present invention such a polyclonal antibody may also be termed “an antibody composition”.

Polyclonal protein/polyclonality: As used herein, the term “polyclonal protein” or “polyclonality” refers to a protein composition comprising different, but homologous protein molecules, preferably selected from the immunoglobulin superfamily. Thus, each protein molecule is homologous to the other molecules of the composition, but also contains one or more stretches of variable polypeptide sequence, which is/are characterized by differences in the amino acid sequence between the individual members of the polyclonal protein. Known examples of such polyclonal proteins include antibody or immunoglobulin molecules, T-cell receptors and B-cell receptors. A polyclonal protein may consist of a defined subset of protein molecules, which has been defined by a common feature such as the shared binding activity towards a desired target, e.g., in the case of a polyclonal antibody against the desired target antigen.

Protein/polypeptide: By “protein” or “polypeptide” is meant any chain of amino acids, regardless of length or post-translational modification. Proteins can exist as monomers or multimers, comprising two or more assembled polypeptide chains, fragments of proteins, polypeptides, oligopeptides, or peptides.

Recombinant antibody: The term “recombinant antibody” is used to describe an antibody molecule or several molecules that is/are expressed from a cell or cell line transfected with an expression vector comprising the coding sequence of the antibody which is not naturally associated with the cell.

Transfection: The term “transfection” is herein used as a broad term for introducing foreign DNA into a cell. The term is also meant to cover other functional equivalent methods for introducing foreign DNA into a cell, such as e.g., transformation, infection, transduction or fusion of a donor cell and an acceptor cell.

Variable polypeptide sequence/variable region: The terms “variable polypeptide sequence” and “variable region” are used interchangeably.

### DESCRIPTION OF THE DRAWINGS

FIG. 1 Sorting of splenocytes (for details see Example 1). The following gates are made (depicted):
Gate 1: Live cells (FSC/Propidium Iodide plot). (Lower left panel)
Gate 2: Plasma cells are gated as CD43 pos/CD138 pos. (lower right panel)
Gate 3: doublet discrimination (upper right panel)

FIG. 2 Murine—mSymplex™ PCR. Multiplex overlap extension RT-PCR for the amplification and cognate linkage of heavy and light chain antibody genes from a single cell. For details refer to Example 1.

FIG. 3 Murine repertoire cloning. A pool of mSymplex™ PCR products encoding VH/VL gene pairs from single plasma cells were spliced to the gene encoding human kappa constant light chain by splicing by overlap extension. The pool of genes, encoding complete human-mouse chimeric antibodies, was inserted in an expression vector followed by an insertion of a bi-directional promoter cassette (2×CMV).

FIG. 4 A schematic representation of the mammalian full-length antibody expression vector 00-VP-002. Amp and Amp pro, ampicillin resistance gene and its promoter; pUC origin, pUC origin of replication; CMV, mammalian promoter driving the expression of the light chain and the heavy chain; IGHV Leader, genomic human heavy chain leader; H stuffer, insert that is exchanged for the heavy chain variable region encoding sequence; IGHG1, sequence encoding for genomic immunoglobulin isotype G1 heavy chain constant region (sequence is shown in Appendix 2); Rabbit B-globin A, rabbit beta-globin polyA sequence; IGKV Leader, murine kappa leader; L Stuffer, insert that is exchanged for the light chain encoding sequence; SV40 term, simian virus 40 terminator sequence; FRT, Flp recognition target site; Neo, neomycin resistance gene; SV40 poly A, simian virus 40 poly A signal sequence.

FIG. 5 Epitope mapping in ELISA. Degree of inhibition of Anti-CD5 antibodies with listed reference antibodies directed against the extra cellular domain of CD5 as determined in a competition ELISA. Scoring of inhibition as follows: 25-49%: Moderate competition (+); 50-74%: Strong competition (++); 75-100%: Very strong competition (+++). * indicates competition experiments which were not performed.

FIG. 6 Epitope maps of anti-CD5 antibodies directed against the extra cellular domain of CD5 as determined by Biacore analysis. A planar view of the binding sites for the generated Anti-CD5 antibodies and the four reference antibodies. The numbers in the figure are antibody numbers corresponding to the antibody numbers indicated elsewhere in this application.

FIG. 7 Sensograms showing simultaneous binding of four antibodies directed against non overlapping epitopes on the extra cellular domain of CD5 as determined by Biacore analysis. A) Sensogram showing the entire experiment. B) Sensogram from A with focus on the simultaneous binding of Anti-CD5 antibodies to the extra cellular domain of CD5. The numbers in the figure are antibody numbers corresponding to the antibody numbers indicated elsewhere in this application.

FIG. 8 Overlay histograms showing simultaneous binding of four antibodies directed against non overlapping epitopes on the extra cellular domain of CD5 as determined by Flow cytometry. The CEM cells were stained as follows (Anti-CD5 antibody, Line, Mean Flourescence Intensity): No antibody, Solid, 7,29; Clone 12, Dash, 479,33; Clone 14, Dot, 636,65; Clone 17, DashDot, 396,29; Clone 34, DashDotDot, 181,14; Mix of Clone 12,14,17 and 34, Solid with Grey fill, 1292,72.

FIG. 9 Mean Fluorescence Intensity (MFI) of cells treated with the indicated antibody mixtures overnight at either 4° C. or 37° C. A decrease in MFI at 37° C. as compared to 4° C. indicates CD5 internalization.

FIG. 10 Western blot analysis of CD5 levels in CLL cells (patient 31) treated with the indicated antibodies and antibody mixtures for the indicated periods of time. Alpha-tubulin is included as loading control.

### DETAILED DESCRIPTION OF THE INVENTION

Antibody Mixtures

In one embodiment, the invention relates to an antibody composition comprising antibody molecules capable of binding at least two distinct CD5 epitopes, preferably two non-overlapping CD5 epitopes. The non-overlapping nature of the antibodies can be determined using differently labelled antibodies in a FACS analysis with CD5 expressing cells or by using Surface Plasmon Resonance using CD5 antigen captured or conjugated to a flow cell surface. ELISA based methods may also be used. A composition binding two non-overlapping CD5 epitopes can be used against a wider range of CD5 expressing cells as it may be less vulnerable to differences in CD5 conformation and less vulnerable to mutations compared to monoclonal antibodies. Furthermore, the antibody composition binding two non-overlapping CD5 epitopes may provide superior efficacy compared to composition targeting a single epitope.

For a monoclonal anti-CD5 antibody therapy a certain proportion of patients will not respond effectively to the antibody treatment. For some of the patients, this may be due to rapid clearing of the antibody or because the antibody generates an immune response in the patient against the antibody. For some patients, the lack of response may be because their particular CD5 expressing cells express CD5 in a conformation where the monoclonal antibody cannot bind its epitope. This could be because of differences in glycosylation, because of domain deletion, or because of mutations and/or SNP(s).

An antibody composition wherein the antibodies are capable of binding at least two distinct epitopes on CD5 will be more broadly applicable, since the likelihood that both epitopes are changed compared to the epitopes recognised by the antibodies is diminished. Furthermore, the likelihood that all antibodies are cleared by the patient is much smaller.

For improved clinical efficacy and broader utility against a wider range of CD5 expressing cell types, the number of antibodies each binding distinct CD5 epitopes in the composition can be increased. Thus, the composition may comprise antibodies capable of binding three non-overlapping epitopes. The composition may comprise antibodies capable of binding four non-overlapping epitopes. The examples of the present application show that at least four distinct antibodies can bind to CD5 at one time. This does not exclude that it is possible or even advantageous to design a composition comprising antibodies capable of binding more than four, such as five, six, seven or eight non-overlapping epitopes by carefully selecting antibodies.

In another embodiment, the composition comprises more than one antibody molecule binding one epitope, such as two antibodies binding different but overlapping epitopes. There may be advantages of including antibodies with overlapping epitopes as this increases the likelihood that the epitope is bound. One rationale behind this is that the epitope in some patients and/or in some cancer cells may be changed due to conformational changes or mutations or SNPs. While this may affect the binding of one antibody, it may not affect the binding of another antibody binding an overlapping epitope. Furthermore, there is a risk that one of the antibodies is cleared by the patients, because it is seen as an antigen. By including two antibodies binding different but overlapping epitopes the consequence of clearance of one of the two antibodies and the consequence of a mutation in an epitope is diminished.

Thus in one embodiment the composition comprises two antibodies binding different but overlapping epitopes. In another embodiment the composition comprises two distinct antibody molecules binding the same epitope. Antibodies binding the same or overlapping epitopes may be of the same or of different isotype.

An antibody composition comprising antibodies directed against two non-overlapping epitopes may thus comprise three, four, five or six distinct antibody molecules so that two antibodies bind two overlapping epitopes or the same first epitope, and another antibody binds a second epitope. Of course, the composition may comprise more than two, such as three or four antibody molecules capable of binding overlapping epitopes or capable of binding the same epitope. Thus the total number of antibodies included in the composition may exceed 6 by having more than one antibody for each epitope or by having several antibodies with overlapping epitopes. Keeping the total dosage of antibody constant, for each further antibody included in the composition, the concentration of each antibody decreases. Therefore it is expected that there is a limit to the number of antibodies that can be included in a composition while maintaining an acceptable efficacy. Based on observations from the Surface Plasmon Resonance binding studies and proliferation assays and taking due account of the manufacture challenges, it is expected that the limited (if any) additional advantage is obtainable by increasing the number of antibodies from 6 to 7, 8, 9, 10 or more. Of course, this does not exclude that the composition comprises more than 10 antibodies, such as 11, 12, 13, 14, 15, 16, 17, 18, 19, or 20 antibodies or more, such as 25 antibodies or more, for example 30 antibodies or more, such as 40 antibodies or more, such as 50 antibodies or more.

Preferably the composition comprises at least one antibody binding a domain I epitope and it may comprise at least two antibodies binding domain I epitopes. Preferably the composition comprises at least one antibody binding a domain II epitope, and it may comprise at least two antibodies binding domain II epitopes. In one embodiment at least one antibody in the composition binds a domain III epitope, more preferably the composition comprises at least two antibodies binding domain III epitopes, and the composition may also comprise three antibodies binding domain III epitopes.

Receptor binding studies have shown that some antibodies may actually stimulate the binding of further antibodies, such that a particular antibody binds in higher quantities to the receptor after receptor saturation with one or several antibodies. When designing the composition of an antibody composition against CD5, antibodies with non-overlapping epitopes are preferably used as these provide a higher synergistic effect.

The antibodies of the composition may be chimeric antibodies with non-human variable chains and human constant chains. The non-human variable chains may be from mouse, rat, sheep, pig, chicken, non-human primate or other suitable animal. In order to obtain fully human antibodies the antibodies can be generated in a transgenic animal with human antibody genes. The antibodies may also be so-called humanised antibodies, where the non-human CDR sequences have been grafted into human framework sequences.

Preferably the human constant chain is IgG1 or IgG2 isotype. More preferably all antibodies in the composition have the same isotype for ease of manufacturing. However, it may be advantageous to include in the composition antibodies of different isotype.

Preferably the antibody compositions of the invention comprise antibodies capable of binding to CD5 selected from the group consisting of human CD5, mutated human CD5, and deletion variants of human CD5. Preferably the antibodies are capable of binding both human and non-human primate CD5, so that they can be tested in relevant toxicology studies prior to clinical experiments. Preferably, the non-human primate is cynomolgous monkey (Macaca fascicularis). Cynomolgous monkey is a relatively small animal, and very well suited for toxicology studies, Therefore, the further primate CD5 is preferably cynomolgous CD5. Preferably the antibodies bind with approximately the same affinity to human and non-human primate CD5.

The present invention has shown superior results in one or more functional assays when combining 2, 3, 4, 5, 6, 7, and 8 antibodies in one composition. While these data provide guidance on selection of the number of antibodies in the composition, they are in no way to be interpreted in a limiting way. The composition may comprise more than 8 antibodies, even though the experimental data only show simultaneous binding of 4 antibodies. There may be other reasons for including more than 6 antibodies in the composition, such as e.g. differences in clearing rate of the antibody members.

A further preferred feature of the antibodies of the compositions is protein homogeneity, so that the antibodies can be purified easily. For the individual antibody members, an ion exchange chromatography profile with one distinct peak is preferred for ease of characterisation. A clear ion exchange chromatography profile is also preferred for ease of characterisation of the final antibody composition. It is also preferable when combining the antibodies that they can be distinguished using ion exchange chromatography, so that the composition with all the antibodies can be characterised in one run.

The antibodies may be or any origin such as human, murine, rabbit, chicken, pig, lama, sheep. The antibodies may also be chimeric as described in the examples or may be humanised, super humanised or reshaped versions thereof using well-known methods described in the art.

An antibody molecule of the present invention may be selected from antibody molecules with the CDRs of the antibodies no. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and 37 as indicated in table 1. The CDRs of these antibody molecules are indicated in table 1, both as amino acid sequences and nucleotide sequences.

Furthermore, the antibody compositions of the invention may preferably comprise one, two, three, four, five, six, or exclusively antibodies selected from antibody molecules with the CDRs of the antibodies no. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and 37 as indicated in table 1.

TABLE 1
Amino acid and DNA sequences of the CDR regions of the antibodies 1-37.

| | | Protein | | | | | |
|---|---|---|---|---|---|---|---|
| | | sequences | SEQ | | SEQ | | SEQ |
| Ab | | VH | ID | | ID | | ID |
| no. | Name | CDRH1 | NO | CDRH2 | NO | CDRH3 | NO |
| 1 | 1D8 | SGYSFTGYTM | 24 | LINPYNGGTT | 49 | CARDYYGSSPDFDYW | 69 |
| 2 | 3I21 | SGYSFTDYTM | 25 | LINPYNGGTM | 50 | CARDNYGSSPDFDYW | 70 |
| 3 | 4H10 | SGYSFTGYTM | 24 | LINPYNGGTM | 50 | CARDNYGSSPYFDYW | 71 |
| 4 | 8J23 | SGYSFTGYTM | 24 | LINPYNGGTM | 50 | CARDNYGSSPYFDYW | 71 |
| 5 | 5O4 | SGYSFTGYTM | 24 | LINPYNGGTT | 49 | CARDYYGSSPDFDYW | 69 |
| 6 | 4H2 | SGFTFSNYAM | 26 | SISSGGNTF | 51 | CVRYYYGVTYWYFDVW | 72 |
| 7 | 5G2 | SGFTFSSYAM | 27 | SISSGGSTY | 52 | CVRYYYGIRYWYFDVW | 73 |
| 8 | 8G8 | SGYSFTAYNI | 28 | SIDPYYGDTK | 53 | CARRMITMGDWYFDVW | 74 |
| 9 | 6M4 | SGYSFTAYSM | 29 | SIDPYYGDTK | 53 | CARRMITTGDWYFDVW | 75 |
| 10 | 2E3 | SGYTFTNFAI | 30 | LISSNSGDVS | 54 | CARHYGAHNYFDYW | 76 |
| 11 | 4E24 | SGYTFTNFAI | 30 | LISTSSGDVS | 55 | CARHYGANNYFDYW | 77 |
| 12 | 4F10 | SGYTFTNFAI | 30 | LISSNSGDVS | 54 | CARHYGAHNYFDYW | 76 |
| 13 | 7J9 | SGYTFTNFAI | 30 | LISSNSGDVS | 54 | CARHYGAHNYFDYW | 76 |
| 14 | 7P9 | SGFNIKDTYM | 31 | RIDPANGNTK | 56 | CAREENYYGTYYFDYW | 78 |
| 15 | 8E24 | SGYSFTSYWM | 32 | MIHPSDSETR | 57 | CARWGDHDDAMDFW | 79 |
| 16 | 6L18 | SGFSLTNYDV | 33 | VIWSGGNTD | 58 | CARNHGDGYFNWYFDVW | 80 |
| 17 | 7H7 | SGFSLTNYDV | 33 | VIWSGGNTD | 58 | CARNHGDGYYNWYFDVW | 81 |
| 18 | 1E7 | SGFTFSNYGM | 34 | AINSNGDITY | 59 | CARGTAWFTYW | 82 |
| 19 | 8J21 | SGYSFTGYTM | 24 | LINPYNGGTR | 60 | CARDGDDGWDIDVW | 83 |
| 20 | 7I11 | SGYIFANYGM | 35 | WINTYTGEPT | 61 | CARRGTYWHFDVW | 84 |
| 21 | 8M9 | SGYNFTNYGM | 36 | WINTYTGEPT | 61 | CARRGSYWHFDVW | 85 |
| 22 | 1P21 | SGYTFTNYGM | 37 | WINTYTGEPT | 61 | CARRSTLVFDYW | 86 |
| 23 | 2H11 | SGYTFTDYYI | 38 | WIYPGGGNTR | 62 | CARNGYWYFDVW | 87 |
| 24 | 3M22 | SGYTFTDYYI | 38 | WIYPGGGNTR | 62 | CARNGYWYFDVW | 87 |
| 25 | 5M6 | SGNTFTNFYL | 39 | CIYPGNVKTK | 63 | CAKEGDYDGTAYFDYW | 88 |
| 26 | 5H8 | SGYTFTNYGM | 37 | WINTYTGEPT | 61 | CARRRDGNFDYW | 89 |
| 27 | 7I19 | SEFTFSNYAM | 40 | TISSGGSYTY | 64 | CVRHGYFDVW | 90 |
| 28 | 1A20 | SGYTFTSYRM | 41 | RIDPYDSGTH | 65 | CAFYDGAYW | 91 |
| 29 | 8E15 | SGFNIKDTYM | 31 | RIDPANGNTK | 56 | CASYDPDYW | 92 |
| 30 | 8C10 | SGYSFTDYTM | 25 | LINPYNGGTR | 60 | CARDTTATYYFDYW | 93 |
| 31 | 3P16 | SGYMFTNHGM | 42 | WINTYTGEPT | 61 | CARRVATYFDVW | 94 |
| 32 | 4F3 | SGYMFTNYGM | 43 | WINTYTGEPT | 61 | CTRRSHITLDYW | 95 |
| 33 | 5M24 | SGYIFTNYGM | 44 | WINTYTGEPT | 61 | CARRRTTAFDYW | 96 |
| 34 | 5O24 | SGFNIKDYYI | 45 | WIDPENGRTE | 66 | CNNGNYVRHYYFDYW | 97 |
| 35 | 7B16 | SGYTFINYGM | 46 | WINTYTGEPT | 61 | CTRRREITFDYW | 98 |
| 36 | 1E8 | SGYTFTDYFI | 47 | EIYPGSSNTY | 67 | CARSGISPFTYW | 99 |
| 37 | 2H16 | SGYIFTGYNI | 48 | AVYPGNGDTS | 68 | CAKYDRGFASW | 100 |
| | | Protein | | | | | |
| | | sequences | SEQ | | SEQ | | SEQ |
| Ab | | VL | ID | | ID | | ID |
| no. | Name | CDRL1 | NO | CDRL2 | NO | CDRL3 | NO |
| 1 | 1D8 | SQGISNHL | 101 | YFTSS | 128 | CQQYSNLPYTF | 151 |
| 2 | 3I21 | SQGIRNYL | 102 | YFTSS | 128 | CQQYSNLPYTF | 151 |
| 3 | 4H10 | SQGISNHL | 101 | YFTSS | 128 | CQQYSNLPYTF | 151 |
| 4 | 8J23 | SQGINNYL | 103 | YYTSS | 129 | CQQYSKIPYTC | 152 |
| 5 | 5O4 | SQGISNHL | 101 | YFTSS | 128 | CQQYSNLPYTF | 151 |
| 6 | 4H2 | SQSVDHDGDSYM | 104 | YAASN | 130 | CQQNYEDPTF | 153 |
| 7 | 5G2 | SQSVDYDGDSYM | 105 | YAASN | 130 | CQQSNEDPTF | 154 |
| 8 | 8G8 | SQDISNYL | 106 | YYTSR | 131 | CQQGDALPWTF | 155 |
| 9 | 6M4 | SQDISTYL | 107 | FYTSR | 132 | CQQGNSLPFTF | 156 |
| 10 | 2E3 | TSSISSSYL | 108 | YGTSN | 133 | CQQWSSRPPTF | 157 |
| 11 | 4E24 | NSSVSSSYL | 109 | YGTSN | 134 | CQQYSGYPLTF | 158 |
| 12 | 4F10 | TSSISSSYL | 108 | YGTSN | 134 | CQQYSDYPLTF | 159 |
| 13 | 7J9 | TSSISSSYL | 108 | YGTSN | 134 | CQQRSYFPFTF | 160 |
| 14 | 7P9 | SENIYYNL | 109 | YNANS | 135 | CKQVYDVPFTF | 161 |
| 15 | 8E24 | SENIYGYF | 110 | YNAKT | 136 | CQHHYGTPFTF | 162 |
| 16 | 6L18 | SQDINNYI | 111 | HYTST | 137 | CLQYDNLWTF | 163 |
| 17 | 7H7 | SQDINKYI | 112 | HYTST | 137 | CLQYDNLWTF | 163 |
| 18 | 1E7 | SENIYSYL | 113 | YNAKT | 136 | CQHHYGYPYTF | 164 |
| 19 | 8J21 | SQGIRNYL | 102 | YHTST | 138 | CQQYSNLPLTF | 165 |
| 20 | 7I11 | SQDVRTDV | 114 | YSASF | 139 | CQQHYTSPWTF | 166 |
| 21 | 8M9 | SQDVITAV | 115 | YSASY | 140 | CQQHYSTPWTF | 167 |
| 22 | 1P21 | SQSIGTSI | 116 | KSASE | 141 | CQQSNRWPLTF | 168 |
| 23 | 2H11 | SSQSLLNQKNYL | 117 | YWAST | 142 | CQNDYDYPYTF | 169 |
| 24 | 3M22 | SSSVSSSYL | 118 | YSTSN | 143 | CHQYHRSPLTF | 170 |
| 25 | 5M6 | SENIYYNL | 109 | YNANS | 135 | CQQTFDVPWTF | 171 |
| 26 | 5H8 | SQTIGTSI | 118 | KNASE | 144 | CQQSNSWPLTY | 172 |
| 27 | 7I19 | SQSLLYSSDQKNYL | 119 | YWAST | 142 | CQQYYNYPLTF | 173 |
| 28 | 1A20 | NSSVSYM | 120 | YDTSK | 135 | CQQWSSNPFTF | 174 |
| 29 | 8E15 | SENIYYNL | 109 | YNANS | 135 | CKQAYDVPWTF | 175 |
| 30 | 8C10 | SSSLSYM | 121 | YDTSN | 146 | CQQWSSFPPTF | 176 |
| 31 | 3P16 | SQRIGTSM | 122 | KSASE | 141 | CQQSNSWPLTF | 177 |
| 32 | 4F3 | SQSIGTSI | 116 | KSASE | 141 | CQQSNSWPLTF | 177 |
| 33 | 5M24 | SQNIGTSI | 123 | KDASE | 147 | CQQSDSWPLTF | 178 |
| 34 | 5O24 | ISSVSYM | 124 | YATSN | 148 | CQQWSSNPRTF | 179 |
| 35 | 7B16 | SQTIATSI | 125 | KNASE | 144 | CQQSNSWPLTF | 177 |
| 36 | 1E8 | SQSLVHSNGNTYL | 126 | YKVSN | 149 | CWQNTHFPQTF | 180 |
| 37 | 2H16 | NESVEYSGTSLM | 127 | SAASN | 150 | CQQSRQVPLTF | 181 |
| | | DNA | | | | | |
| | | sequences | | SEQ | | SEQ | |
| Ab | | VH | | ID | | ID | |
| no. | Name | V gene | CDRH1 | NO | CDRH2 | NO | |
| 1 | 1D8 | IGHV1S135*01 | tctggttactcattcactggctacaccatg | 182 | cttattaatccttacaatggtggtactacc | 210 | |
| 2 | 3I21 | IGHV1S59*01 | tcaggttactcattcactgactacaccatg | 183 | cttattaatccttacaatggtggtactatg | 211 | |
| 3 | 4H10 | IGHV1S135*01 | tcaggttattcattcaccggctacaccatg | 184 | cttattaatccttacaatggtggtactatg | 211 | |
| 4 | 8J23 | IGHV1S8*01 | tcaggttattcattcaccggctacaccatg | 184 | cttattaatccttacaatggtggtactatg | 211 | |
| 5 | 5O4 | IGHV1S9*01 | tctggttactcattcactggctacaccatg | 182 | cttattaatccttacaatggtggtactacc | 210 | |
| 6 | 4H2 | IGHV5S9*01 | tctggattcactttcagtaactatgccatg | 185 | tccattagtagtggtggtaacaccttt | 212 | |
| 7 | 5G2 | IGHV5S9*01 | tctggattcactttcagtagctatgccatg | 186 | tccattagtagtggtggtagcacc | 213 | |
| 8 | 8G8 | IGHV1S135*01 | tctggttattcattcactgcctacaacatt | 187 | agtattgatccttactatggtgatactaaa | 214 | |
| 9 | 6M4 | IGHV1S135*01 | tctggttactcattcactgcctacagcatg | 188 | agtattgatccttattatggtgatactaag | 215 | |
| 10 | 2E3 | IGHV1S137*01 | tctggctacacattcactaattttgctatt | 189 | cttattagttctaactctggtgatgttagc | 216 | |
| 11 | 4E24 | IGHV1S137*01 | tctggctacacattcactaattttgctatt | 189 | cttattagtacttcctctggtgatgttagc | 217 | |
| 12 | 4F10 | IGHV1S137*01 | tctggctacacattcactaattttgctatt | 189 | cttattagttctaactctggtgatgttagc | 216 | |
| 13 | 7J9 | IGHV1S137*01 | tctggctacacattcactaattttgctatt | 189 | cttattagttctaactctggtgatgttagc | 216 | |
| 14 | 7P9 | IGHV14S1*01 | tctggcttcaacattaaagacacctatatg | 190 | aggattgatcctgcgaatggtaatactaaa | 218 | |
| 15 | 8E24 | IGHV1S6*01 | tctggctactccttcaccagttactggatg | 191 | atgattcatccttccgatagtgaaactagg | 219 | |
| 16 | 6L18 | IGHV2S2*01 | tctggtttctcattaactaactatgatgta | 192 | gtgatttggagtggtggaaacacagac | 220 | |
| 17 | 7H7 | IGHV2S2*01 | tctggtttctcattaactaactatgatgta | 192 | gtgatatggaattatggaaacacagac | 221 | |
| 18 | 1E7 | IGHV5S10*01 | tctggattcactttcagtaactatggcatg | 193 | gccattaatagtaatggtgatattacc | 222 | |
| 19 | 8J21 | IGHV1S135*01 | tctggttactcattcactggctacaccatg | 182 | cttattaatccttacaatggtggtactaga | 223 | |
| 20 | 7I11 | IGHV9S3*02 | tctgggtatattttcgcaaactatggcatg | 194 | tggataaacacctacactggagagccaaca | 224 | |
| 21 | 8M9 | IGHV9S3*02 | tctgggtataacttcacaaactatggaatg | 195 | tggattaacacctacactggagagccaaca | 225 | |
| 22 | 1P21 | IGHV9S3*02 | tctggttataccttcacaaactatggaatg | 196 | tggataaacacttacactggagagccaaca | 226 | |
| 23 | 2H11 | IGHV1S125*01 | tctggcgacaccgtcactgactacgatata | 197 | tggatttatcctggaggcggtaatactagg | 227 | |
| 24 | 3M22 | IGHV1S125*01 | tctggctacaccttcactgactactatata | 197 | tggatttatcctggaggcggtaatactagg | 227 | |
| 25 | 5M6 | IGHV1S50*01 | tctggcaacaccttcacaaacttctattta | 198 | tgtatttatcctggaaacgttaagactaaa | 228 | |
| 26 | 5H8 | IGHV9S3*02 | tctgggtataccttcacaaactatggaatg | 199 | tggataaacacctacactggagagcctaca | 229 | |
| 27 | 7I19 | IGHV5S9*01 | tctgaattcactttcagtaactatgccatg | 200 | accattagtagtggtggtagttacacc | 230 | |
| 28 | 1A20 | IGHV1S6*01 | tctggctacacgttcaccagctacaggatg | 201 | aggattgatccttacgatagtggaactcac | 231 | |
| 29 | 8E15 | K3HV14S1*01 | tctggcttcaacattaaagacacctatatg | 190 | aggattgatcctgcgaatggtaatactaaa | 218 | |
| 30 | 8C10 | IGHV1S135*01 | tctggttactcattcactgactacaccatg | 202 | cttattaatccttacaatggtggtactagg | 232 | |
| 31 | 3P16 | IGHV9S3*02 | tctgggtatatgttcacaaaccatggaatg | 203 | tggataaacacctacactggagagccaaca | 224 | |
| 32 | 4F3 | IGHV9S3*02 | tctgggtatatgttcacaaactatggaatg | 204 | tggataaacacctacactggagagccaaca | 224 | |
| 33 | 5M24 | IGHV9S3*02 | tctgggtatatcttcacaaactatggaatg | 205 | tggataaacacctacactggagagccaaca | 224 | |
| 34 | 5O24 | IGHV14S3*01 | tctggcttcaacattaaagactactatata | 206 | tggattgatcctgagaatggtcgtactgaa | 233 | |
| 35 | 7B16 | IGHV9S3*02 | tctgggtataccttcataaattatggaatg | 207 | tggataaacacctacactggagagccaaca | 224 | |
| 36 | 1E8 | IGHV1S125*01 | tctggctacaccttcactgactactttata | 208 | gagatttatcctggaagtagtaatacttac | 234 | |
| 37 | 2H16 | IGHV1S50*01 | tctggctacatttttaccggttacaatata | 209 | gctgtttatccaggaaatggtgatacttcc | 235 | |
| | | DNA | | | | | |
| | | sequences | | SEQ | | | |
| Ab | | VH | | ID | | | |
| no. | Name | V gene | CDRH3 | NO | | | |
| 1 | 1D8 | IGHV1S135*01 | tgtgcaagagattactacggtagtagtccagactttgactactgg | 236 | | | |
| 2 | 3I21 | IGHV1S59*01 | tgtgcaagagataactacggtagtagtccagactttgactactgg | 237 | | | |
| 3 | 4H10 | IGHV1S135*01 | tgtgcaagagataactacggtagtagcccatactttgactactgg | 238 | | | |
| 4 | 8J23 | IGHV1S8*01 | tgtgcaagagataactacggtagtagcccatactttgactactgg | 238 | | | |
| 5 | 5O4 | IGHV1S9*01 | tgtgcaagagattactacggtagtagtccagactttgactactgg | 236 | | | |
| 6 | 4H2 | IGHV5S9*01 | tgtgtccgttattactacggtgttacctactggtacttcgatgtctgg | 239 | | | |
| 7 | 5G2 | IGHV5S9*01 | tgtgtccgttattactacggtattaggtactggtacttcgatgtctgg | 240 | | | |
| 8 | 8G8 | IGHV1S135*01 | tgtgcaagaaggatgattacgatgggagactggtatttcgatgtctgg | 241 | | | |
| 9 | 6M4 | IGHV1S135*01 | tgtgcaagaaggatgattacgacgggagactggtacttcgatgtctgg | 242 | | | |
| 10 | 2E3 | IGHV1S137*01 | tgtgcaagacactatggtgcccacaactattttgactattgg | 243 | | | |
| 11 | 4E24 | IGHV1S137*01 | tgtgcaagacactatggtgccaacaactattttgactattgg | 244 | | | |
| 12 | 4F10 | IGHV1S137*01 | tgtgcaagacactatggtgcccacaactattttgactattgg | 243 | | | |
| 13 | 7J9 | IGHV1S137*01 | tgtgcaagacactatggtgcccacaactattttgactattgg | 243 | | | |
| 14 | 7P9 | IGHV14S1*01 | tgtgctagagaggagaattactacggtacctactactttgactactgg | 245 | | | |
| 15 | 8E24 | IGHV1S6*01 | tgtgcaagatggggggatcacgacgatgctatggacttctgg | 246 | | | |
| 16 | 6L18 | IGHV2S2*01 | tgtgccagaaatcatggtgatggttacttcaactggtacttcgatgtctgg | 247 | | | |
| 17 | 7H7 | IGHV2S2*01 | tgtgccagaaatcatggtgatggttactataactggtacttcgatgtctgg | 248 | | | |
| 18 | 1E7 | IGHV5S10*01 | Tgtgcaagaggaactgcctggtttacttactgg | 249 | | | |
| 19 | 8J21 | IGHV1S135*01 | tgtgcaagagatggggatgatggttgggacatcgatgtctgg | 250 | | | |
| 20 | 7I11 | IGHV9S3*02 | tgtgcaagaagggggacttactggcacttcgatgtctgg | 251 | | | |
| 21 | 8M9 | IGHV9S3*02 | tgtgcaagaagggggtcttactggcacttcgatgtctgg | 252 | | | |
| 22 | 1P21 | IGHV9S3*02 | Tgtgcaagacgctctacgctcgtctttgactactgg | 253 | | | |
| 23 | 2H11 | IGHV1S125*01 | Tgtgcaagaaacggctactggtacttcgatgtctgg | 254 | | | |
| 24 | 3M22 | IGHV1S125*01 | Tgtgcaagaaacggctactggtacttcgatgtctgg | 254 | | | |
| 25 | 5M6 | IGHV1S50*01 | tgtgcaaaggagggagattacgacgggacggcctactttgattactgg | 255 | | | |
| 26 | 5H8 | IGHV9S3*02 | Tgtgcaagaaggcgggacggaaactttgactactgg | 256 | | | |
| 27 | 7I19 | IGHV5S9*01 | Tgtgtaagacatggatacttcgatgtctgg | 257 | | | |
| 28 | 1A20 | IGHV1S6*01 | Tgtgccttctatgatggggcttactgg | 258 | | | |
| 29 | 8E15 | IGHV14S1*01 | Tgtgctagttatgatcctgactactgg | 259 | | | |
| 30 | 8C10 | IGHV1S135*01 | tgtgcaagagatactacggcgacgtactactttgactactgg | 260 | | | |
| 31 | 3P16 | IGHV9S3*02 | Tgtgcaagacgtgttgcgacgtacttcgatgtctgg | 261 | | | |
| 32 | 4F3 | IGHV9S3*02 | Tgtacacgaaggagtcatattaccttggactactgg | 262 | | | |
| 33 | 5M24 | IGHV9S3*02 | Tctgggtatatcttcacaaactatggaatg | 205 | | | |
| 34 | 5O24 | IGHV14S3*01 | tgtaataatggtaactacgtcagacactactactttgactactgg | 263 | | | |
| 35 | 7B16 | IGHV9S3*02 | Tgtacaagaagaagagaaataacctttgactactgg | 264 | | | |
| 36 | 1E8 | IGHV1S125*01 | Tgtgcaagatcggggatttcgccctttacttactgg | 265 | | | |
| 37 | 2H16 | IGHV1S50*01 | Tgtgcaaaatatgaccgggggtttgcttcctgg | 266 | | | |
| | | DNA | | | | | |
| | | sequences | | SEQ | | SEQ | |
| Ab | | VL | | ID | | ID | |
| no. | Name | V gene | CDRL1 | NO | CDRL2 | NO | |
| 1 | 1D8 | IGKV10-94*01 | Agtcagggcattagcaatcattta | 267 | tatttcacatcaagt | 297 | |
| 2 | 3I21 | IGKV10-94*01 | Agtcagggcattagaaattattta | 268 | tatttcacatcaagt | 297 | |
| 3 | 4H10 | IGKV10-94*01 | Agtcagggcattagcaatcattta | 267 | tatttcacatcaagt | 297 | |
| 4 | 8J23 | IGKV10-94*01 | Agtcagggcattaacaattattta | 269 | tattacacatcaagt | 298 | |
| 5 | 5O4 | IGKV10-94*01 | Agtcagggcattagcaatcattta | 267 | tatttcacatcaagt | 297 | |
| 6 | 4H2 | IGKV3-4*01 | Agccaaagtgttgatcatgatggtgatagttatatg | 270 | tatgctgcatccaat | 299 | |
| 7 | 5G2 | IGKV3-4*01 | Agccaaagtgttgattatgatggtgatagttatatg | 271 | tatgctgcatccaat | 299 | |
| 8 | 8G8 | IGKV10-96*01 | Agtcaggacattagcaattattta | 272 | tactacacatcaaga | 300 | |
| 9 | 6M4 | IGKV10-96*01 | Agtcaggacattagtacttattta | 273 | ttctacacatcacga | 301 | |
| 10 | 2E3 | IGKV4-73*01 | Acctcaagtataagttccagttacttg | 274 | tatggcacatccaac | 302 | |
| 11 | 4E24 | IGKV4-78*01 | Aactcaagtgtaagttccagttacttg | 275 | tatggcacatccaac | 302 | |
| 12 | 4F10 | IGKV4-78*01 | Acctcaagtataagttccagttacttg | 274 | tatggcacatccaac | 302 | |
| 13 | 7J9 | IGKV4-79*01 | Acctcaagtataagttccagttacttg | 274 | tatggcacatccaac | 302 | |
| 14 | 7P9 | IGKV12-38*01 | Agtgagaacatttactacaattta | 276 | tataatgcaaacagc | 303 | |
| 15 | 8E24 | IGKV12-44*01 | Agtgaaaatatttacggttatttc | 277 | tataatgcaaaaacc | 304 | |
| 16 | 6L18 | IGKV19-93*01 | Agtcaagacattaacaactatata | 278 | cattacacatctaca | 305 | |
| 17 | 7H7 | IGKV19-93*01 | Agtcaagacattaacaagtatata | 279 | cattacacatctaca | 305 | |
| 18 | 1E7 | IGKV12-44*01 | Agtgagaatatttacagttattta | 280 | tataatgcaaaaacc | 304 | |
| 19 | 8J21 | IGKV10-94*01 | Agtcagggcattagaaattattta | 268 | tatcacacatcaact | 306 | |
| 20 | 7I11 | IGKV6-17*01 | Agtcaggatgtgaggactgatgta | 281 | tactcggcatccttc | 307 | |
| 21 | 8M9 | IGKV6-17*01 | Agtcaggatgtgattactgctgta | 282 | tactcggcatcctac | 308 | |
| 22 | 1P21 | IGKV5-48*01 | Agtcagagcattggtacaagcata | 283 | aagagtgcttctgag | 309 | |
| 23 | 2H11 | IGKV8-19*01 | Tccagtcagagtctcttaaatcaaaagaactacttg | 284 | actgggcatccact | 310 | |
| 24 | 3M22 | IGKV4-74*01 | Agctcaagtgtaagttccagttacttg | 284 | tatagcacatccaac | 311 | |
| 25 | 5M6 | IGKV12-38*01 | Agtgagaacatttactacaattta | 286 | tataatgcaaacagt | 312 | |
| 26 | 5H8 | IGKV5-48*01 | Agtcagaccattggcacaagcata | 286 | aagaatgcttctgag | 313 | |
| 27 | 7I19 | IGKV8-30*01 | agtcagagccttttatatagtagcgatcaaaagaactacttg | 287 | tactgggcatccact | 310 | |
| 28 | 1A20 | IGKV4-59*01 | Aactcaagtgtaagttacatg | 288 | tatgacacatccaaa | 314 | |
| 29 | 8E15 | IGKV12-38*01 | Agtgagaacatttactacaattta | 276 | tataatgcaaacagc | 303 | |
| 30 | 8C10 | IGKV4-55*01 | Agctcaagtttaagttacatg | 289 | tatgacacatccaac | 315 | |
| 31 | 3P16 | IGKV5-48*01 | Agtcagcgcattggcacaagcatg | 290 | aagtctgcttctgag | 316 | |
| 32 | 4F3 | IGKV5-48*01 | Agtcagagcattggcacaagcata | 291 | aagtctgcttctgag | 316 | |
| 33 | 5M24 | IGKV5-48*01 | Agtcagaacattggcacaagcata | 292 | aaggatgcttctgag | 317 | |
| 34 | 5O24 | IGKV4-72*01 | Atctcaagtgtaagttacatg | 293 | tatgccacttccaac | 318 | |
| 35 | 7B16 | IGKV5-48*01 | Agtcagaccattgccacaagcata | 294 | aagaatgcttctgag | 313 | |
| 36 | 1E8 | IGKV1-110*01 | agtcagagccttgtacacagtaatggaaacacctattta | 295 | tacaaagtttccaat | 319 | |
| 37 | 2H16 | IGKV3-1*01 | Aatgaaagtgttgaatattctggcacaagtttaatg | 296 | tctgctgcatccaac | 320 | |
| | | DNA | | | | | |
| | | sequences | | SEQ | | | |
| Ab | | VL | | ID | | | |
| no. | Name | V gene | CDRL3 | NO | | | |
| 1 | 1D8 | IGKV10-94*01 | Tgtcagcagtatagtaaccttccgtacacgttc | 321 | | | |
| 2 | 3I21 | IGKV10-94*01 | Tgtcagcagtatagtaaccttccgtacacgttc | 321 | | | |
| 3 | 4H10 | IGKV10-94*01 | Tgtcagcagtatagtaaccttccgtacacgttc | 321 | | | |
| 4 | 8J23 | IGKV10-94*01 | Tgtcagcagtatagtaagattccgtacacgtgc | 322 | | | |
| 5 | 5O4 | IGKV10-94*01 | Tgtcagcagtatagtaaccttccgtacacgttc | 321 | | | |
| 6 | 4H2 | IGKV3-4*01 | Tgtcagcaaaattatgaggatccgacgttc | 323 | | | |
| 7 | 5G2 | IGKV3-4*01 | Tgtcagcaaagtaatgaggatccgacgttc | 324 | | | |
| 8 | 8G8 | IGKV10-96*01 | Tgtcaacagggtgatgcgcttccgtggacgttc | 325 | | | |
| 9 | 6M4 | IGKV10-96*01 | Tgccaacagggtaattcgcttccgttcacgttc | 326 | | | |
| 10 | 2E3 | IGKV4-73*01 | tgccagcagtggagtagtagaccacccacgttc | 327 | | | |
| 11 | 4E24 | IGKV4-78*01 | Tgccagcagtacagtggttacccactcacgttc | 328 | | | |
| 12 | 4F10 | IGKV4-78*01 | Tgccagcagtacagtgattacccactcacgttc | 329 | | | |
| 13 | 7J9 | IGKV4-79*01 | Tgccagcaaaggagttatttcccgttcacgttc | 330 | | | |
| 14 | 7P9 | IGKV12-38*01 | Tgtaaacaggtttatgacgttccattcacgttc | 331 | | | |
| 15 | 8E24 | IGKV12-44*01 | Tgtcaacatcattatggtactccattcacgttc | 332 | | | |
| 16 | 6L18 | IGKV19-93*01 | Tgtctacagtatgataatctgtggacgttc | 333 | | | |
| 17 | 7H7 | IGKV19-93*01 | Tgtctacagtacgataatctgtggacgttc | 334 | | | |
| 18 | 1E7 | IGKV12-44*01 | Tgtcaacatcattatggttatccgtatacgttc | 335 | | | |
| 19 | 8J21 | IGKV10-94*01 | Tgtcagcagtatagtaaccttccgctcacgttc | 336 | | | |
| 20 | 7I11 | IGKV6-17*01 | Tgtcagcaacattatacttctccgtggacgttc | 337 | | | |
| 21 | 8M9 | IGKV6-17*01 | Tgtcagcaacattatagtactccgtggacgttc | 338 | | | |
| 22 | 1P21 | IGKV5-48*01 | tgtcaacaaagtaataggtggccgctcacgttc | 339 | | | |
| 23 | 2H11 | IGKV8-19*01 | Tgtcagaatgattatgattatccttacacgttc | 340 | | | |
| 24 | 3M22 | IGKV4-74*01 | Tgccaccagtatcatcgttccccgctcacgttc | 341 | | | |
| 25 | 5M6 | IGKV12-38*01 | Tgtcaacagacttttgacgttccgtggacgttc | 342 | | | |
| 26 | 5H8 | IGKV5-48*01 | tgtcaacaaagtaatagctggccactcacgtac | 343 | | | |
| 27 | 7I19 | IGKV8-30*01 | Tgtcagcaatattataactatccgctcacgttc | 344 | | | |
| 28 | 1A20 | IGKV4-59*01 | Tgccagcagtggagtagtaacccattcacgttc | 345 | | | |
| 29 | 8E15 | IGKV12-38*01 | Tgtaaacaggcttatgacgttccgtggacgttc | 346 | | | |
| 30 | 8C10 | IGKV4-55*01 | Tgccagcagtggagtagtttcccaccgacattc | 347 | | | |
| 31 | 3P16 | IGKV5-48*01 | Tgtcaacaaagtaatagttggccgctcacgttc | 348 | | | |
| 32 | 4F3 | IGKV5-48*01 | Tgtcaacaaagtaatagctggccgctcacgttc | 349 | | | |
| 33 | 5M24 | IGKV5-48*01 | Tgtcaacaaagtgatagctggccactcacgttc | 350 | | | |
| 34 | 5O24 | IGKV4-72*01 | tgccagcagtggagtagtaacccacggacgttc | 351 | | | |
| 35 | 7B16 | IGKV5-48*01 | Tgtcaacaaagtaatagctggccactcacgttc | 352 | | | |
| 36 | 1E8 | IGKV1-110*01 | Tgctggcaaaatacacattttcctcagacgttc | 353 | | | |
| 37 | 2H16 | IGKV3-1*01 | Tgtcagcaaagtaggcaggttcctctcacgttc | 354 | | | |

TABLE 2
VL chain amino acid sequence of each antibody (listed by antibody name in
the format “>[antibody number], SEQ ID NO: [NO]” followed by the sequence).

>19, SEQ ID NO: 355
NIVLTQSTSSLSASLGDRVTISCSASQGIRNYLNWYQQKPDGTVKLLIYHTSTLHSGVP
SRFSGSGSGTDYSLTISNLEPEDIATYYCQQYSNLPLT
>12, SEQ ID NO: 356
DIVLTQSPAIMSASPGEQVTMTCRATSSISSSYLHWYQQKSGASPKLWIYGTSNLASG
VPTRFSGSGSGTSYSLTISSVEAEDAATYYCQQYSDYPLT
>15, SEQ ID NO: 357
DIVLTQSPASLSASVGESVTITCRPSENIYGYFAWYQQRQGKSPQLLVYNAKTLAEGV
PSRFSGSGSGTHFSLKINSLQPEDFGTYYCQHHYGTPFT
>29, SEQ ID NO: 358
DIVLTQSPASLAASVGETVTITCRASENIYYNLAWYQQKQGKSPQLLIYNANSLEGGVP
SRFSGSGSGTQYSMKINSMQPEDTATYFCKQAYDVPWT
>30, SEQ ID NO: 359
EIVLTQSPAIMSASPGEKVTMTCSASSSLSYMYWYQQKPGSSPRLLIYDTSNLASGVP
FRFSGSGSGTSYSLTISRMEAEDAATYYCQQWSSFPPT
>13, SEQ ID NO: 360
EIVLTQSPAIMSASPGEQVTMTCRATSSISSSYLHWYQQKSGASPKLWIYGTSNLASG
VPTRFSGGGSGTSYSLTISRMEAEDAATYYCQQRSYFPFT
>27, SEQ ID NO: 361
NIVMTQSPSSLAVSVGEKVTMSCKSSQSLLYSSDQKNYLAWYQLKPGQSPKLLIYWA
STRESGVPDRFTGSGSGTDFTLTISSVKAEDLAVYYCQQYYNYPLT
>14, SEQ ID NO: 362
NIVLTQSPASLAASVGETVTITCRASENIYYNLAWYQQKQGKSPQLLIYNANSLEDGVP
SRFSGSGSGTQYSMKINSMQPEDTATYFCKQVYDVPFT
>35, SEQ ID NO: 363
HIVLTQSPAILSVSPGERVSFSCRASQTIATSINWYQQRTNGSPRLLIKNASESISGIPS
RFSGSGSGTDFTLTINSVESEDIADYYCQQSNSWPLT
>9, SEQ ID NO: 364
HIVLTQSPSSLSASLGDRVTISCRASQDISTYLNWYQQKPDGTVKLLIFYTSRLHAGVP
SRFSGSGSGTHHSLTISNLEQEDIATYFCQQGNSLPFT
>16, SEQ ID NO: 365
DIVMTQSPSSLSESLGGKVTITCKASQDINNYIAWYQHKPGKGPRLLIHYTSTLLPGIPS
RFSGSGSGTDYSFSISNLEPEDIATYYCLQYDNLWT
>34, SEQ ID NO: 366
DVVLTQSPAILSASPGEKVTMTCRAISSVSYMHWYQQKPGSSPKPWIYATSNLASGV
PARFSGSGSGTSYSLTISRVEAEDAATYYCQQWSSNPRT
>5, SEQ ID NO: 367
NIVLTQSTSSLSASLGDRVTINCSASQGISNHLNWFQQKSDGTVKLLIYFTSSLHSGVP
SRFSGSWSGTDYSLTISNLEPEDIAAYYCQQYSNLPYT
>33, SEQ ID NO: 368
NIVLTQSPAILSVSPGERVSFSCRASQNIGTSIHWYQQRTNGSPRFLVKDASESISGIP
SRFSGSGSGTDFTLTINNVESEDIADYYCQQSDSWPLT
>25, SEQ ID NO: 369
NIVLTQSPASLAASVGETVTITCRVSENIYYNLAWYQQKQGKSPQLLIYNANSLEDGVP
SRFSGSGSGTQYSMKINSMQPEDTATYFCQQTFDVPWT
>26, SEQ ID NO: 370
HIVLTQSPAILSVSPGERVSFSCRASQTIGTSIHWYQQRTNGSPRLLIKNASESISGIPS
RFSGSGSGTDFTLSINSVESEDIADYYCQQSNSWPLT
>7, SEQ ID NO: 371
QIVLTQSPASLPASPGQRATISCKASQSVDYDGDSYMNWYHQKPGQPPKLLIYAASN
LESGIPARFSGSGSGTDFTLNIHPVEEEDAATYYCQQSNEDPT
>3, SEQ ID NO: 372
NIVLTQSTSSLSASLGDRVTINCSASQGISNHLNWFQQKSDGTVKLLIYFTSSLHSGVP
SRFSGSGSGTDYSLTISNLEPEDIAAYYCQQYSNLPYT
>6, SEQ ID NO: 373
NIVLTQSPASLAVSLGQRATISCKASQSVDHDGDSYMNWYQQKPGQSPKLLTYAASN
LDSGIPARFSGSGSRTDFTLNIHPVEEEDAATYYCQQNYEDPT
>8, SEQ ID NO: 374
EIVLTQSPSSLSASLGDRVTISCRASQDISNYLNWYQRKPDGTVKLLIYYTSRLQSGVP
SRFSGSGSGSEYSLTISNLDQEDIATYFCQQGDALPWT
>32, SEQ ID NO: 375
DIVLTQSPVILSVSPGERVSLSCRASQSIGTSINWYQQRTDGSPRLLIKSASESMSGIP
SRFSGSGSGTDFTLSITSVESEDIADYYCQQSNSWPLT
>11, SEQ ID NO: 376
EIVLTQSPTIMSASPGEQVTMTCRTNSSVSSSYLHWYQQKSGASPKLWIYGTSNLAS
GVPTRFSGSGSGTSYSLTISSVEAGDAATYFCQQYSGYPLT
>31, SEQ ID NO: 377
NIVLTQSPAILSVSPGERVSFSCRASQRIGTSMNWYQQRTNGSPRLLIKSASESISGIP
SRFSGSGSGTDFTLSINSVESDDVADYYCQQSNSWPLT
>24, SEQ ID NO: 378
DIVMTQSPAIMSASLGERVTMTCTASSSVSSSYLHWYQQKPGSSPKLWIYSTSNLAS
GVPARFSGSGSGTSYSLTISSMEAEDAATYYCHQYHRSPLT
>2, SEQ ID NO: 379
NIVLTQSTSSLSASLGDRVTISCSASQGIRNYLNWYQQKSDGTVKLLIYFTSSLHSGVP
SRFSGSGSGTDYSLTISNLEPEDIAAYYCQQYSNLPYT
>37, SEQ ID NO: 380
NIVLTQSPASLAVSLGQRATISCRVNESVEYSGTSLMQWYQQKPGQPPKLLISAASNV
ESGVPARFSGRGSGTDFSLNIHPVEEDDIAMYFCQQSRQVPLT
>23, SEQ ID NO: 381
DIVLTQSPSSLTVTAGEKVTMSCKSSQSLLNQKNYLTWYQQKTGQPPKLLIYWASTRE
SGVPDRFTGSGSGTDFTLTISSVQAEDLAVYYCQNDYDYPYT
>10, SEQ ID NO: 382
NIVMTQSPAIMSASPGEQVTMTCRATSSISSSYLHWYQQKSGASPKLWIYGTSNLAS
GVPTRFSGSGSGTSYSLTISSMEAEDAATYYCQQWSSRPPT
>22, SEQ ID NO: 383
NIVMTQSPAILSVSPGERVSFSCRASQSIGTSINWYQQRTNASPRLLIKSASESISGIPS
RFSGSGSGTDFTLNIKNVESEDIADYYCQQSNRWPLT
>36, SEQ ID NO: 384
MFVMTQTPLSLPVSLGDQASISCRSSQSLVHSNGNTYLHWYLQKPGQSPKLLIYKVS
NRFSGVPDRFSGSGSGTDFTLKISRVEAEDLGVYYCWQNTHFPQT
>18, SEQ ID NO: 385
DIVLTQSPASLSASVGETVIITCRASENIYSYLVWYQQKQGKSPQLLVYNAKTLAEGVP
SRFSGSGSGTQFSLKINSLQSEDFGSYSCQHHYGYPYT
>1, SEQ ID NO: 386
DIVLTQSTSSLSASLGDRVTINCSASQGISNHLNWFQQKSDGTVKLLIYFTSSLHSGVP
SRFSGSGSGTDYSLTISNLEPEDIAAYYCQQYSNLPYT
>4, SEQ ID NO: 387
DIVMTQSTSSLSASLGDRVTISCSASQGINNYLNWYQQKPDGTVKLLIYYTSSLHSGV
PPRFSGSGSGTDYSLTISNLEPEDIATYYCQQYSKIPYT
>21, SEQ ID NO: 388
HIVLTQSHKFMSTSVGDRVSITCKASQDVITAVTWSQQKPGQSPKLLIYSASYRYTGV
PDRFTGSGSGTDFTFTISSVQAEDLAVYYCQQHYSTPWT
>28, SEQ ID NO: 389
DIVLTQSPAIMSASPGEKVTMTCSANSSVSYMLWYQQKSGTSPKRWIYDTSKLSSGV
PARFSGSGSGTSYSLTISSMEAEDAATYYCQQWSSNPFT
>20, SEQ ID NO: 390
NIVMTQSHRFMSTSVGDRVSITCKASQDVRTDVAWFQQKPGQSPKLLIYSASFRYTG
VPDRFTGSGSGTDFTLTISSVQAEDLAVYYCQQHYTSPWT
>17, SEQ ID NO: 391
NIVLTQSPSSLSESLGGKVTITCKASQDINKYIAWYQYKPGKGPRLLIHYTSTLQPGIPS
RFSGSGSGRDYSFSISNLEPEDIATYYCLQYDNLWT

TABLE 3
VH chain amino acid sequence of each antibody (listed by antibody name in
the format “>[antibody number], SEQ ID NO: [NO]” followed by the sequence).

>16, SEQ ID NO: 392
EVKLVESGPGLVQPSQSLSITCTVSGFSLTNYDVHWVRQSPGKGLEWLGVIWSGGN
TDYNAAFISRLSITKDNSKSQVFFKMNSLQTKDTAIYSCARNHGDGYFNWYFDV
>17, SEQ ID NO: 393
EVQLVESGPGLVQPSQSLSITCTVSGFSLTNYDVHWVRQSPGKGLEWLGVIWNYGN
TDYNAAFISRLSIRKDSSKSQVFFTMSSLQTPDTAIYYCARNHGDGYYNWYFDV
>27, SEQ ID NO: 394
EVQLVESGGGLVKPGGSLKLSCAASEFTFSNYAMSWVRQTPEKGLEWVATISSGGS
YTYYSDSVKGRFTISRDNVKNTLYLQMSSLRSEDTAMYYCVRHGYFDV
>7, SEQ ID NO: 395
EVQLVESGGGLVKPGGSLKLSCAASGFTFSSYAMSWVRQTPEKRLEWVASISSGGS
TYYPDTVKGRFTISRDNARNILYLQMSSLRSEDTAMYYCVRYYYGIRYWYFDV
>6, SEQ ID NO: 396
QVQLQESGGVLVKPGGSLKLSCAASGFTFSNYAMSWVRQTPEKRLEWVASISSGGN
TFYPDNVKGRFTISRDNSRNILYLQMTSLRSEDSAMYYCVRYYYGVTYWYFDV
>18, SEQ ID NO: 397
QVQLKESGGGLVQPGGSLKLSCAASGFTFSNYGMSWVRQIPDKRLELVAAINSNGDI
TYDPDSVKGRFTISRDNANNSLFLQMRSLKSEDTAMYYCARGTAWFTYWGQGTLVTV
>19, SEQ ID NO: 398
EVQLQESGPELVKPGASMKISCKASGYSFTGYTMNWVKQSHGENLEWIGLINPYNG
GTRYNQKFKDKATLTVNKSSSTAYMELLSLTSEDSAVYYCARDGDDGWDIDV
>4, SEQ ID NO: 399
QVQLQESGPELVKPGASMKISCKASGYSFTGYTMNWVKQSHGKNLEWIGLINPYNG
GTMYNQKFKGKATLTVDKSSNTAYMELLSLTSEDSAVYYCARDNYGSSPYFDY
>28, SEQ ID NO: 400
EVQLQQPEAELVRPGASVKLSCKASGYTFTSYRMNWVKQRPEEGLEWIGRIDPYDS
GTHYNQKFKDKAILTVDKSSSIAYMQLSSLTSEDSAVYYCAFYDGAY
>1, SEQ ID NO: 401
EVQLQESGPELVKPEASVKISCKASGYSFTGYTMNWVKQSHGKNLEWIGLINPYNGG
TTYNQKFKGKATLTVDTSSSTAFMELLSLTSEDSAVYYCARDYYGSSPDFDY
>20, SEQ ID NO: 402
EVKLVESGPELKKPGETVKISCKASGYIFANYGMNWVKQAPGKGLKWMGWINTYTG
EPTYADDFKGRFAFSLETSASTARLQINNLKKEDTATYFCARRGTYWHFDV
>8, SEQ ID NO: 403
QVQLKESGPELEKPGASVRISCKASGYSFTAYNINWVTQRDGKSLEWIGSIDPYYGDT
KYNQKFKDKATLTVDKSSSTAHMQVKSLTSEDSAIYYCARRMITMGDWYFDV
>22, SEQ ID NO: 404
QVQLQESGPELKKPGETVKISCKASGYTFTNYGMNWVKQAPGKGLKWMGWINTYTG
EPTYADDFKGRFALSLEASVSTAYLQINNLKNEDTATYFCARRSTLVFDY
>10, SEQ ID NO: 405
QVQLKESGAELVRPGVSVKISCKGSGYTFTNFAIHWVKQSHAKSLEWIGLISSNSGDV
SYNQKFKGKATMTVDKSSSTAYMELARLTSEDSAIYYCARHYGAHNYFDY
>15, SEQ ID NO: 406
QVTLKESGAELVRPGASVKLSCKASGYSFTSYWMNWVKQRPGQGLEWIGMIHPSDS
ETRLNQKFKDKATLTVDKSSSTAYMQLSSPTSEDSAVYYCARWGDHDDAMDF
>29, SEQ ID NO: 407
QVQLKESGADLVKPGASVKLSCTASGFNIKDTYMNWVKERPEQGLEWIGRIDPANGN
TKYDPKFQGKATITADTSSNTGYLQLSSLTSEDTAVYYCASYDPDY
>30, SEQ ID NO: 408
EVQLVESGPELVRPGASMRISCKASGYSFTDYTMNWVKQSHGKNLEWIGLINPYNGG
TRNNQKFKGKATLTVDKSSSTAYMELLSLTSEDSAVYYCARDTTATYYFDY
>23, SEQ ID NO: 409
EVQLQQSGPELVKPGTSVKISCKASGYTFTDYYINWVKQKPGQGLEWIGWIYPGGGN
TRYIERFKGKATLTVDTSSSTAYMQLSSLTSEDTAVYFCARNGYWYFDV
>21, SEQ ID NO: 410
EVQLQQSGPELKKPGETVKISCKASGYNFTNYGMNWVKQAPGKGLKWMGWINTYT
GEPTYADDFKGRFAFSLETSASTVYLRINNLKNEDSSTFFCARRGSYWHFDV
>2, SEQ ID NO: 411
EVQLQQPGPELVKPGASMKISCKASGYSFTDYTMNWVKQSHGKNLEWIGLINPYNG
GTMYNQKFKDKATLTVDKSSNTAYMELLSLTSEDSAVYYCARDNYGSSPDFDY
>24, SEQ ID NO: 412
EVKLVESGPELVKPGTSVKISCKASGYTFTDYYINWVKQRPGQGLEWIGWIYPGGGN
TRYIERFKGKATLTVDTSSSTAYMQLSSLTSEDTAVYFCARNGYWYFDV
>31, SEQ ID NO: 413
EVQLKESGPELKKPGETVKISCMTSGYMFTNHGMNWVKQAPGKGLKWMGWINTYT
GEPTYGDGFKGRFVFSLETSASTAYLQINNLKNEDTATYFCARRVATYFDV
>11, SEQ ID NO: 414
EVQLKESGAELVRPGGSVKISCKGSGYTFTNFAIHWVKQSHIKTLEWIGLISTSSGDVS
YNQKFKDKATMTVDKSSSTAYMELARLTSEDSAIYYCARHYGANNYFDY
>32, SEQ ID NO: 415
EVKLVESGPELKKPGETVKISCKASGYMFTNYGMNWVKQAPGKGLKWMGWINTYTG
EPTYVEDFKGRFAFSLETSANTAYLQINNLKNEDTATYFCTRRSHITLDY
>12, SEQ ID NO: 416
EVQLQESGAELVRPGVSVKISCKGSGYTFTNFAIHWVKQSHAKSLEWIGLISSNSGDV
SYNQKFKGKATMTVDKSSSTAYMELARLTSEDSAIYYCARHYGAHNYFDY
>14, SEQ ID NO: 417
EVQLKESGAELVKPGASVKLSCTASGFNIKDTYMHWVKQRPEQGLEWIGRIDPANGN
TKYDPKFQGKATITADTSSNTAYLQLSSLTSEDTAVYYCAREENYYGTYYFDY
>3, SEQ ID NO: 418
QVQLKESGPELVKPGASMKISCKASGYSFTGYTMNWVKQSHGKNLEWIGLINPYNG
GTMYNQKFKGKATLTVDKSSNTAYMELLSLTSEDSAVYYCARDNYGSSPYFDY
>26, SEQ ID NO: 419
KVQLQQSGPELKKPGETVKISCKASGYTFTNYGMNWVKQAPGKGLKWMGWINTYTG
EPTYADDFKGRFAFSLETSARTAYLQINNLKNEDSATYFCARRRDGNFDY
>25, SEQ ID NO: 420
EVKLVESGPELVKPGASVRISCKSSGNTFTNFYLHWMKQRPGQGLEWIGCIYPGNVK
TKYSARFKGKAILTADKSSSTVFMQLSNLTSEDSAVYFCAKEGDYDGTAYFDY
>33, SEQ ID NO: 421
QVTLKESGPELKKPGETVKISCRASGYIFTNYGMNWVKQAPGKGLKWMGWINTYTG
EPTYADDFKGRFAFSLETSASTAHLQINNLKNEDTAIYFCARRRTTAFDY
>5, SEQ ID NO: 422
EVKLVESGPELVKPEASVKISCKASGYSFTGYTMNWVKQSHGKNLEWIGLINPYNGG
TTYNQKFKGKATLTVDTSSSTAFMELLSLTSEDSAVYYCARDYYGSSPDFDY
>34, SEQ ID NO: 423
EVKLVESGAELVRSGASVKLSCAASGFNIKDYYIHWVKQRPEQGLEWIGWIDPENGR
TEYAPKFQGKATMTADTSSNTAYLQLSSLTSEDTAVYYCNNGNYVRHYYFDY
>35, SEQ ID NO: 424
QVQLQQPGPELKKPGETVKISCKASGYTFINYGMNWVKQAPGKGLKWMGWINTYTG
EPTYADDFKGRFAFSLETSASTAYLQINNLEHEDMAVYFCTRRREITFDY
>13, SEQ ID NO: 425
QVQLQQSGAELVRPGVSVKISCKGSGYTFTNFAIHWVKQSHAKSLEWIGLISSNSGDV
SYNQKFKGKATMTVDKSSSTAYMELARLTSEDSAIYYCARHYGAHNYFDY
>9, SEQ ID NO: 426
QVQLKESGPELEKPGASVKISCKASGYSFTAYSMNWVKQNNGMSLEWIGSIDPYYGD
TKYAQKFKGKATLTVDKASSTAYLQLKSLTSEDSAVYYCARRMITTGDWYFDV
>36, SEQ ID NO: 427
QVQLQQPGAELARPGASVMLSCKASGYTFTDYFINWVKQRTGQGLDWIGEIYPGSS
NTYYNEKFKGKATLTADESSSTAYMRLSSLTSEDSAV*FCARSGISPFTY
>37, SEQ ID NO: 428
QVQLKESGADLVKPGASVKMSCKTSGYIFTGYNIHWVKQTPGQGLVWIGAVYPGNG
DTSYNQNFKAKATLTADISSTTAYMQLSSLTSEDSAIYYCAKYDRGFAS

TABLE 4
VL chain nucleotide sequence of each antibody (listed by antibody name in
the format “>[antibody number], SEQ ID NO: [NO]” followed by the sequence).

>19, SEQ ID NO: 429
aacattgtgctgacccagtctacatcctccctgtctgcctctctgggagacagagtcaccatcagttgc
agtgcaagtcagggcattagaaattatttaaactggtatcagcagaaaccagatggaactgttaaactcc
tgatctatcacacatcaactttacactcaggagtcccatcaaggttcagtggcagtgggtctgggacaga
ttattctctcaccatcagcaacctggaacctgaagatattgccacttactattgtcagcagtatagtaac
cttccgctcacg
>12, SEQ ID NO: 430
gacattgtgctgacccagtctccagcaatcatgtctgcatctccaggggagcaggtcaccatgacctgc
agggccacctcaagtataagttccagttacttgcactggtaccagcagaagtccggtgcctcccccaaac
tctggatttatggcacatccaacttggcttctggagtccctactcgcttcagtggcagtgggtctgggac
ctcttactctctcacaatcagcagtgtggaggctgaagatgctgccacttattactgccagcagtacagt
gattacccactcacg
>15, SEQ ID NO: 431
gacattgtgctgacccaatctccagcctccctatctgcatctgtgggagaatctgtcaccatcacatgt
cggccaagtgaaaatatttacggttatttcgcatggtatcagcagagacagggaaaatctcctcagctcc
tggtctataatgcaaaaaccttagcagaaggtgtgccatcaaggttcagtggcagtggatcaggcacaca
tttttctctgaagatcaacagcctacagcctgaagattttgggacttattactgtcaacatcattatggt
actccattcacg
>29, SEQ ID NO: 432
gacattgtgctgactcagtctccagcctccctggctgcatctgtgggagaaactgtcaccatcacatgt
cgagcaagtgagaacatttactacaatttagcatggtatcagcagaagcaagggaaatctcctcagctcc
tgatctataatgcaaacagcttggaaggtggtgtcccatcgaggttcagtggcagtggatctgggacaca
gtattctatgaagatcaacagcatgcagcctgaagacaccgcaacttatttctgtaaacaggcttatgac
gttccgtggacg
>30, SEQ ID NO: 433
gaaattgtgctgacccagtctccagcaatcatgtctgcatctccaggggagaaggtcaccatgacctgc
agtgccagctcaagtttaagttacatgtattggtaccagcagaagccaggatcctcccccagactcctga
tttatgacacatccaacctggcttctggagtcccttttcgcttcagtggcagtgggtctgggacctctta
ctctctcacaatcagccgaatggaggctgaagatgctgccacttattactgccagcagtggagtagtttc
ccaccgaca
>13, SEQ ID NO: 434
gaaattgttctgacccagtctccagcaatcatgtctgcatctccaggggagcaggtcaccatgacctgc
agggccacctcaagtataagttccagttacttgcactggtaccagcagaagtccggtgcctcccccaaac
tctggatttatggcacatccaacttggcttctggagtccctactcgcttcagtggcggtgggtctgggac
ctcttactctctcacaatcagccgaatggaggctgaagatgctgccacttattactgccagcaaaggagt
tatttcccgttcacg
>27, SEQ ID NO: 435
aacattgtgatgacacagtctccatcctccctagctgtgtcagttggagagaaggttactatgagctgc
aagtccagtcagagccttttatatagtagcgatcaaaagaactacttggcctggtaccagctgaaaccag
ggcagtctcctaaactgctgatttactgggcatccactagggaatctggggtccctgatcgcttcacagg
cagtggatctgggacagatttcactctcaccatcagcagtgtgaaggctgaagacctggcagtttattac
tgtcagcaatattataactatccgctcacg
>14, SEQ ID NO: 436
aacattgtgctgactcagtctccagcctccctggctgcatctgtgggagaaactgtcaccatcacatgt
cgagcaagtgagaacatttactacaatttagcatggtatcagcagaagcaagggaaatctcctcagctcc
tgatctataatgcaaacagcttggaagatggtgtcccatcgaggttcagtggcagtggatctgggacaca
gtattctatgaagatcaacagcatgcagcctgaagataccgcaacttatttctgtaaacaggtttatgac
gttccattcacg
>35, SEQ ID NO: 437
cacattgttctgacccaatctccagccatcctgtctgtgagtccaggggagagagtcagtttctcctgc
agggccagtcagaccattgccacaagcataaactggtatcagcaaagaacaaatggttctccaaggcttc
tcataaagaatgcttctgagtctatctctgggatcccttccaggtttagtggcagtggatcagggacaga
ttttactcttaccatcaacagtgtggagtctgaagatattgcagattattactgtcaacaaagtaatagc
tggccactcacg
>9, SEQ ID NO: 438
cacattgttctgacccaatctccatcttccctgtctgcctctctgggagacagagtcaccatcagttgc
agggcaagtcaggacattagtacttatttaaactggtatcagcagaaaccggatggaactgttaaactcc
tgatcttctacacatcacgattacacgcaggagtcccatcaaggttcagtggcagtgggtctggaacaca
tcattctctcaccattagcaacctggaacaagaagatattgccacttacttttgccaacagggtaattcg
cttccgttcacg
>16, SEQ ID NO: 439
gatattgtgatgacacagtctccatcctcactgtctgaatctctgggaggcaaagtcaccatcacatgc
aaggcaagtcaagacattaacaactatatagcttggtaccaacacaagcctggaaaaggtcctaggctgc
tcatacattacacatctacattgctgccaggcatcccatcaaggttcagtggaagtgggtctgggacaga
ttattccttcagcatcagcaacctggagcctgaagatattgcaacttattattgtctacagtatgataat
ctgtggacg
>34, SEQ ID NO: 440
gatgttgtgctgacccaatctccagcaatcctgtctgcatcgccaggggagaaggtcacaatgacttgc
agggccatctcaagtgtaagttacatgcactggtaccagcagaagccaggatcatcccccaaaccctgga
tttatgccacttccaacctggcttctggagtccctgctcgcttcagtggcagtgggtctgggacctctta
ctctctcacaatcagcagagtggaggctgaagatgctgccacttattactgccagcagtggagtagtaac
ccacggacg
>5, SEQ ID NO: 441
aacattgtgctgacacagtctacatcctccctgtctgcctctctgggagacagagtcaccatcaattgc
agtgcaagtcagggcattagcaatcatttaaactggtttcagcagaaatcagatggaactgttaaactcc
tgatctatttcacatcaagtttacactcgggagtcccttcaaggttcagtggcagttggtctgggacaga
ttattctctcaccatcagcaacctggaacctgaagatattgccgcttactattgtcagcagtatagtaac
cttccgtacacg
>33, SEQ ID NO: 442
aacattgtgctgacacagtctccagccatcctgtctgtgagtccaggggagagagtcagtttctcctgc
agggccagtcagaacattggcacaagcatacactggtatcagcaaagaacaaatggttctccgaggtttc
tcgtaaaggatgcttctgagtctatctctgggatcccttccaggtttagtggcagtggatcagggacaga
ttttactcttaccatcaacaatgtggagtctgaagatattgcagattattactgtcaacaaagtgatagc
tggccactcacg
>25, SEQ ID NO: 443
aacattgtgctgactcagtctccagcctccctggctgcatctgtgggagaaactgtcaccatcacatgt
cgagtaagtgagaacatttactacaatttagcatggtatcagcagaagcaagggaaatctcctcagctcc
tgatctataatgcaaacagtttggaagatggtgtcccatcgaggttcagtggcagtggatctggaacaca
gtattctatgaagatcaacagcatgcagcctgaagataccgcaacttatttctgtcaacagacttttgac
gttccgtggacg
>26, SEQ ID NO: 444
cacattgtgctcacccaatctccagccatcctatctgtgagtccaggagagagagtcagtttctcctgc
agggccagtcagaccattggcacaagcatacactggtatcagcaaagaacaaatggttctccaaggcttc
tcataaagaatgcttctgagtctatctctgggatcccttccaggtttagtggcagtggatcagggacaga
ttttactcttagcatcaacagtgtggagtctgaagatattgcagattattactgtcaacaaagtaatagc
tggccactcacg
>7, SEQ ID NO: 445
caaattgttctcacccagtctccagcttctttgcctgcgtctccaggacagagggccaccatctcctgc
aaggccagccaaagtgttgattatgatggtgatagttatatgaactggtaccatcagaaaccaggacagc
cacccaaactcctcatctatgctgcatccaatctcgaatctgggatcccagccaggtttagtggcagtgg
gtctgggacagacttcaccctcaacatccatcctgtggaggaggaggatgctgcaacctattactgtcag
caaagtaatgaggatccgacg
>3, SEQ ID NO: 446
aacattgtgctgactcagtctacatcctccctgtctgcctctctgggagacagagtcaccatcaattgc
agtgcaagtcagggcatiagcaatcatttaaactggtttcagcagaaatcagatggaactgttaaactcc
tgatctatttcacatcaagtttacactcgggagtcccttcaaggttcagtggcagtgggtctgggacaga
ttattctctcaccatcagcaacctggaacctgaagatattgccgcttactattgtcagcagtatagtaac
cttccgtacacg
>6, SEQ ID NO: 447
aacattgtgctgacccagtctccagcttctttggctgtgtctctaggacagagggccaccatctcctgc
aaggccagccaaagtgttgatcatgatggtgatagttatatgaactggtaccaacagaaaccaggacagt
cacccaaactcctcacctatgctgcatccaatctagattctgggatcccagccaggtttagtggcagtgg
gtctcggacagacttcaccctcaacatccaccctgtggaggaggaggatgctgcaacctattactgtcag
caaaattatgaggatccgacg
>8, SEQ ID NO: 448
gaaattgttctcacccagtctccatcctccctgtctgcctctctgggagacagagtcaccatcagttgc
agggcaagtcaggacattagcaattatttaaactggtatcagcggaaaccagatgggactgttaaactcc
tgatctactacacatcaagattacagtcaggagtcccatcaaggttcagtggcagtgggtctggttcaga
gtattctctcaccattagcaacctggaccaagaggatattgccacttacttttgtcaacagggtgatgcg
cttccgtggacg
>32, SEQ ID NO: 449
gacattgtgctgacacagtctccagtcatcctgtctgtgagcccaggagaaagagtcagtctctcctgc
agggccagtcagagcattggcacaagcataaattggtatcagcagagaacagatggttctccaaggcttc
tcataaagtctgcttctgagtctatgtctgggatcccttccaggtttagtggcagtggatcagggacaga
ttttactcttagcatcaccagtgtggagtctgaagatattgcagattattactgtcaacaaagtaatagc
tggccgctcacg
>11, SEQ ID NO: 450
gaaattgttctcacccaatctccaacaatcatgtctgcttctccaggggagcaggtcaccatgacctgc
cggaccaactcaagtgtaagttccagttacttgcactggtaccagcagaagtcaggtgcctcccccaaac
tctggatttatggcacatccaacttggcttctggagtccctactcgtttcagtggcagtgggtctgggac
ctcttactctctcacaatcagcagtgtggaggctggagatgctgccacttatttctgccagcagtacagt
ggttacccactcacg
>31, SEQ ID NO: 451
aacattgtgctgacccagtctccagccatcctgtctgtgagtccaggagagagagtcagtttctcctgc
agggccagtcagcgcattggcacaagcatgaactggtatcaacaaagaacaaatggttctccaaggcttc
tcataaagtctgcttctgagtctatctctgggatcccttccaggtttagtggcagcggttcagggacaga
ttttactcttagcatcaacagtgtggagtctgacgatgttgcagattattactgtcaacaaagtaatagt
tggccgctcacg
>24, SEQ ID NO: 452
gacattgtgatgacacagtctccagcaatcatgtctgcatctctaggggaacgggtcaccatgacctgc
actgccagctcaagtgtaagttccagttacttgcactggtaccagcagaagccaggatcctcccccaaac
tctggatttatagcacatccaacctggcttctggagtcccagctcgcttcagtggcagtgggtctgggac
ctcttactctctcacaatcagcagcatggaagctgaagatgctgccacttattactgccaccagtatcat
cgttccccgctcacg
>2, SEQ ID NO: 453
aacattgtgctgacccagtctacatcctccctgtctgcctctctgggagacagagtcaccatcagttgc
agtgcaagtcagggcattagaaattatttaaactggtatcagcagaaatcagatggaactgttaaactcc
tgatctatttcacatcaagtttacactcgggagtcccttcaaggttcagtggcagtgggtctgggacaga
ttattctctcaccatcagcaacctggaacctgaagatattgccgcttactattgtcagcagtatagtaac
cttccgtacacg
>37, SEQ ID NO: 454
aacattgtgctgactcagtctccagcttctttggctgtatctctagggcagagagccaccatctcctgc
agagtcaatgaaagtgttgaatattctggcacaagtttaatgcagtggtaccaacagaaaccaggacagc
cacccaaactcctcatctctgctgcatccaacgtagaatctggggtccctgccaggtttagtggccgtgg
gtctgggacagacttcagcctcaacatccatcctgtggaggaggatgatattgcaatgtatttctgtcag
caaagtaggcaggttcctctcacg
>23, SEQ ID NO: 455
gacattgtgctgacacagtctccatcctccctgactgtgacagcaggagagaaggtcactatgagctgc
aagtccagtcagagtctcttaaatcaaaagaactacttgacctggtaccagcagaaaacagggcagcctc
ctaaactgttgatctactgggcatccactagggaatctggggtccctgatcgcttcacaggcagtggatc
tggaacagatttcactctcaccatcagcagtgtgcaggctgaagacctggcagtttattactgtcagaat
gattatgattatccttacacg
>10, SEQ ID NO: 456
aacattgtgatgactcagtctccagcaatcatgtctgcatctccaggggagcaggtcaccatgacctgc
agggccacctcaagtataagttccagttacttgcactggtaccagcagaagtccggtgcctcccccaaac
tctggatttatggcacatccaacttggcttctggagtccctactcgcttcagtggcagtgggtctgggac
ctcttactctctcacaatcagcagtatggaggctgaagatgctgccacttattactgccagcagtggagt
agtagaccacccacg
>22, SEQ ID NO: 457
aacattgtgatgactcagtctccagccattctgtctgtgagtccaggagaaagagtcagcttctcctgc
agggccagtcagagcattggtacaagcataaactggtatcagcaaagaacaaatgcttctccaaggcttc
tcataaagagtgcttctgagtctatctctgggatcccttccaggtttagtggcagtggatcagggacaga
ttttactcttaacatcaaaaatgtggagtctgaagatattgcagattattactgtcaacaaagtaatagg
tggccgctcacg
>36, SEQ ID NO: 458
atgtttgtgatgacgcaaactccactctccctgcctgtcagtcttggagatcaagcctccatctcttg
cagatctagtcagagccttgtacacagtaatggaaacacctatttacattggtacctacagaagccaggc
cagtctccaaagctcctgatctacaaagtttccaatcgattttctggggtcccagacaggttcagtggca
gtggatcagggacagatttcacactcaagatcagcagagtggaggctgaggatctgggagtttattattg
ctggcaaaatacacattttcctcagacg
>18, SEQ ID NO: 459
gacattgtgctgacacagtctccagcctccctatctgcatctgtgggagaaactgtcatcatcacgtgt
cgagcaagtgagaatatttacagttatttagtatggtatcagcagaaacagggaaaatctcctcagctcc
tggtctataatgcaaaaaccttagcagaaggtgtgccatcaaggttcagtggcagtggatcaggcacaca
gttttctctgaagatcaacagcctgcagtctgaagattttgggagttattcctgtcaacatcattatggt
tatccgtatacg
>1, SEQ ID NO: 460
gacattgtgctgactcagtctacatcctccctgtctgcctctctgggagacagagtcaccatcaattgc
agtgcaagtcagggcattagcaatcatttaaactggtttcagcagaaatcagatggaactgttaaactcc
tgatctatttcacatcaagtttacactcgggagtcccttcaaggttcagtggcagtgggtctgggacaga
ttattctctcaccatcagcaacctggaacctgaagatattgccgcttactattgtcagcagtatagtaac
cttccgtacacg
>4, SEQ ID NO: 461
gacattgtgatgacccagtctacatcctccctgtctgcctctctgggagacagagtcaccatcagttgc
agtgcaagtcagggcattaacaattatttaaactggtatcagcagaaaccagatggaactgttaaactcc
tgatctattacacatcaagtttacactcaggagtcccaccaaggttcagtggcagtgggtctgggacaga
ttattctctcaccatcagcaacctggaacctgaagatattgccacttactattgtcagcagtatagtaag
attccgtacacg
>21, SEQ ID NO: 462
cacattgtgctgacccaatctcacaaattcatgtccacatcagtaggagacagggtcagcatcacctgc
aaggccagtcaggatgtgattactgctgtaacctggtctcaacagaaaccaggacaatctcctaaactac
tgatttactcggcatcctaccggtacactggagtccctgatcgcttcactggcagtggatctgggacgga
tttcactttcaccatcagcagtgtacaggctgaagacctggcagtttattactgtcagcaacattatagt
actccgtggacg
>28, SEQ ID NO: 463
gacattgttctgacccagtctccagcaatcatgtctgcatctccaggggagaaggtcaccatgacctgc
agtgccaactcaagtgtaagttacatgctctggtaccagcagaagtcaggcacctcccccaaaagatgga
tttatgacacatccaaactgtcttctggagtccctgctcgcttcagtggcagtgggtctgggacctctta
ctctctcacaatcagcagcatggaggctgaagatgctgccacttattactgccagcagtggagtagtaac
ccattcacg
>20, SEQ ID NO: 464
aacattgtgatgacccagtctcacagattcatgtccacatcagtaggagacagggtcagcatcacctgc
aaggccagtcaggatgtgaggactgatgtagcctggtttcaacagaaaccaggacaatctcctaaactac
tgatttactcggcatccttccggtacactggagtccctgaccgcttcactggcagtggatctgggacgga
tttcactctcaccatcagcagtgtgcaggctgaagacctggcagtttattactgtcagcaacattatact
tctccgtggacg
>17, SEQ ID NO: 465
aacattgtgctgacacagtctccatcctcactgtctgaatctctgggaggcaaagtcaccatcacatgt
aaggcaagtcaagacattaacaagtatatagcttggtaccaatacaagcctggaaaaggtcctaggctgc
tcatacattacacatctacattacagccaggcatcccatcaaggttcagtggaagtggttctgggagaga
ttattccttcagcatcagcaacctggagcctgaagatattgcaacttattattgtctacagtacgataat
ctgtggacg

TABLE 5
VH chain nucleotide sequence of each antibody (listed by antibody name in
the format “>[antibodyname], SEQ ID NO: [NO]” followed by the sequence}.

>16, SEQ ID NO: 466
gaagtgaagcttgttgagtcaggacctggcctagtgcagccctcacagagcctgtccatcacctgcacag
tctctggtttctcattaactaactatgatgtacactgggttcgccagtctccaggaaagggtctggagtg
gctgggagtgatttggagtggtggaaacacagactataatgcagctttcatatccagactgagcatcacc
aaggacaattccaagagccaagttttctttaaaatgaacagtctgcaaactaaagacacagccatatact
cctgtgccagaaatcatggtgatggttacttcaactggtacttcgatgtc
>17, SEQ ID NO: 467
gaggtgcagctggttgagtcaggacctggcctagtgcagccctcacagagcctgtccatcacctgcacag
tctctggtttctcattaactaactatgatgtacactgggttcgccagtctccaggaaagggtctggagtg
gctgggagtgatatggaattatggaaacacagactataatgcagctttcatatccagactgagcatcagg
aaggacagttccaagagccaagttttctttacaatgagcagtctgcaaactcctgacacagccatatatt
actgtgccagaaatcatggtgatggttactataactggtacttcgatgtc
>27, SEQ ID NO: 468
gaggtgcagcttgtggagtctgggggaggcttagtgaagcctggagggtccctgaaactctcctgtgcag
cctctgaattcactttcagtaactatgccatgtcttgggttcgccagactccggagaagggcctggagtg
ggtcgcaaccattagtagtggtggtagttacacctactattcagacagtgtgaagggtcgattcaccatc
tccagagacaatgtcaagaacaccctgtatctgcaaatgagcagtctgaggtctgaggacacggccatgt
attactgtgtaagacatggatacttcgatgtc
>7, SEQ ID NO: 469
gaagtgcagcttgttgagtctgggggaggcttagtgaagcctggagggtccctgaaactctcctgtgcag
cctctggattcactttcagtagctatgccatgtcttgggttcgccagactccagagaagaggctggagtg
ggtcgcatccattagtagtggtggtagcacctactatccagacactgtgaagggccgattcaccatctcc
agagataatgccaggaacatcctgtacctgcaaatgagcagtctgaggtctgaggacacggccatgtatt
actgtgtccgttattactacggtattaggtactggtacttcgatgtc
>6, SEQ ID NO: 470
caggtgcagcttcaggagtctgggggagtcttagtgaagcctggagggtccctgaaactctcctgtgcag
cctctggattcactttcagtaactatgccatgtcttgggttcgccagactccagagaagaggctggagtg
ggtcgcgtccattagtagtggtggtaacaccttttatccagacaatgtgaagggccgattcaccatctcc
agagataattccaggaacatcctgtacctgcaaatgaccagtctgaggtctgaggactcggccatgtatt
actgtgtccgttattactacggtgttacctactggtacttcgatgtc
>18, SEQ ID NO: 471
caggtgcagcttaaggagtctgggggaggcttagtgcagcctggagggtccctgaaactctcctgtgcag
cctctggattcactttcagtaactatggcatgtcttgggttcgccagattccagacaagaggctggaatt
ggtcgcagccattaatagtaatggtgatattacctatgatccagacagtgtgaagggccgattcaccatc
tccagagacaatgccaacaactccctgttcctgcaaatgagaagtctgaagtctgaggacacagccatgt
attactgtgcaagaggaactgcctggtttacttactggggccaagggactctggtcactgt
>19, SEQ ID NO: 472
gaggtgcagcttcaggagtctggacctgagctggtgaagcctggagcttcaatgaagatatcctgcaagg
cttctggttactcattcactggctacaccatgaactgggtgaagcagagccatggagagaaccttgagtg
gattggacttattaatccttacaatggtggtactagatacaaccagaagttcaaggacaaggccacatta
actgtaaacaagtcatccagcacagcctacatggagctcctcagtctgacatctgaggactctgcagtct
attactgtgcaagagatggggatgatggttgggacatcgatgtc
>4, SEQ ID NO: 473
caggtgcagcttcaggagtctggacctgagctggtgaagcctggagcctcaatgaagatatcctgcaagg
cttcaggttattcattcaccggctacaccatgaactgggtgaagcagagccatggaaagaaccttgagtg
gattggacttattaatccttacaatggtggtactatgtacaaccagaagttcaagggcaaggccacatta
actgtagacaagtcatccaatacagcctacatggagctcctcagtctgacatctgaggactctgcagtct
attactgtgcaagagataactacggtagtagcccatactttgactac
>28, SEQ ID NO: 474
gaggtccaactgcaacagcctgaggctgagctggtgaggcctggggcttcagtgaagctgtcctgcaagg
cttctggctacacgttcaccagctacaggatgaactgggttaagcagaggcctgaggaaggccttgagtg
gattggaaggattgatccttacgatagtggaactcactacaatcaaaagttcaaggacaaggccatattg
actgtagacaaatcctccagtatagcctacatgcaactcagcagcctgacatctgaggactctgcggtct
attactgtgccttctatgatggggcttac
>1, SEQ ID NO: 475
gaggtgcagctgcaggagtctggacctgagctggtgaagcctgaagcttcagtgaagatatcctgcaagg
cttctggttactcattcactggctacaccatgaactgggtgaagcagagccatggaaagaaccttgaatg
gattggacttattaatccttacaatggtggtactacctacaaccagaagttcaagggcaaggccacatta
actgtagacacgtcatccagcacagccttcatggagctcctcagtctgacatctgaagactctgcagtct
attactgtgcaagagattactacggtagtagtccagactttgactac
>20, SEQ ID NO: 476
gaggtgaagcttgttgagtctggacctgagctgaagaagcctggagagacagtcaagatctcctgcaagg
cttctgggtatattttcgcaaactatggcatgaactgggtgaagcaggctccaggaaagggtttaaagtg
gatgggctggataaacacctacactggagagccaacatatgctgatgacttcaagggacggtttgccttc
tctttggaaacctctgccagcactgcccgtttgcagatcaacaacctcaaaaaagaggacacggctacat
atttctgtgcaagaagggggacttactggcacttcgatgtc
>8, SEQ ID NO: 477
caggtgcagctgaaggagtctggacctgaactggagaagcctggcgcttcagtgaggatttcctgcaagg
cttctggttattcattcactgcctacaacattaactgggtgacgcagcgcgatggaaagagccttgagtg
gattggaagtattgatccttactatggtgatactaaatacaaccagaagttcaaggacaaggccacgttg
actgtagacaaatcctccagcacagcccacatgcaggtcaagagcctcacatctgaggactctgcaatct
attactgtgcaagaaggatgattacgatgggagactggtatttcgatgtc
>22, SEQ ID NO: 478
caggtgcagctgcaggagtctggacctgagctgaaaaagcctggagagacagtcaagatctcctgcaagg
cttctggttataccttcacaaactatggaatgaactgggtgaagcaggctccaggaaagggtttaaagtg
gatgggctggataaacacttacactggagagccaacatatgctgatgacttcaagggacggtttgccctc
tctttggaagcctctgtcagcactgcctatttgcagatcaacaacctcaaaaatgaagacacggctacat
atttctgtgcaagacgctctacgctcgtctttgactac
>10, SEQ ID NO: 479
caggtgcagcttaaggagtctggggctgaactggtgaggcctggggtctcagtgaagatttcctgcaagg
gttctggctacacattcactaattttgctattcactgggtgaaacagagtcatgcaaagagtctagagtg
gattggacttattagttctaactctggtgatgttagctacaaccagaagttcaagggcaaggccacaatg
actgtagacaaatcctccagcacagcctatatggaacttgccagactgacatctgaggattctgccatct
attattgtgcaagacactatggtgcccacaactattttgactat
>15, SEQ ID NO: 480
caggttactctgaaagagtctggggctgagctggtgaggcctggagcttcagtgaagctgtcctgcaagg
cttctggctactccttcaccagttactggatgaactgggtgaaacagaggcctggacaaggccttgaatg
gattggcatgattcatccttccgatagtgaaactaggttaaatcagaagttcaaggacaaggccacattg
actgtagacaagtcctccagcacagcctacatgcaactcagcagcccgacatctgaggactctgcggtct
attactgtgcaagatggggggatcacgacgatgctatggacttc
>29, SEQ ID NO: 481
caggtgcagctgaaggagtctggggcagaccttgtgaagccaggggcctcagtcaagttgtcctgcacag
cttctggcttcaacattaaagacacctatatgaactgggtgaaggagaggcctgaacagggcctggagtg
gattggaaggattgatcctgcgaatggtaatactaaatatgacccgaagttccagggcaaggccactata
acagcagacacatcctccaatacaggctacctgcagctcagcagcctgacatctgaggacactgccgtct
attactgtgctagttatgatcctgactac
>30, SEQ ID NO: 482
gaggtgcagctggttgagtctggacctgagctggtgaggcctggagcttcaatgaggatatcctgcaagg
cttctggttactcattcactgactacaccatgaactgggtgaagcagagccatggaaagaaccttgagtg
gattggacttattaatccttacaatggtggtactaggaacaaccagaagttcaagggcaaggccacatta
actgtagacaagtcatccagcacagcctacatggagctcctcagtctgacatctgaggactctgcagtct
attactgtgcaagagatactacggcgacgtactactttgactac
>23, SEQ ID NO: 483
gaggtccaactgcaacagtctggacctgagctggtgaagcctgggacttcagtgaagatatcctgcaagg
cttctggctacaccttcactgactactatataaactgggtgaagcagaagcctggacagggacttgagtg
gattggatggatttatcctggaggcggtaatactaggtacattgagaggttcaagggcaaggccacattg
actgtagacacatcctccagcacagcctacatgcagctcagcagcctaacatctgaggacactgctgtct
atttctgtgcaagaaacggctactggtacttcgatgtc
>21, SEQ ID NO: 484
gaagtccagctgcaacagtctggacctgagctgaagaagcctggagagacagtcaagatctcctgcaagg
cttctgggtataacttcacaaactatggaatgaactgggtgaagcaggctccaggaaagggtttaaagtg
gatgggctggattaacacctacactggagagccaacatatgctgatgacttcaagggacggtttgccttc
tctttggaaacctctgccagcactgtctatttgcggatcaacaacctcaaaaatgaggactcgtctacat
ttttctgtgcaagaagggggtcttactggcacttcgatgtc
>2, SEQ ID NO: 485
gaggtccaactgcaacagcctggacctgagctggtgaagcctggagcctcaatgaagatatcctgcaagg
cttcaggttactcattcactgactacaccatgaactgggtgaaacagagccatggaaagaaccttgagtg
gattggacttattaatccttacaatggtggtactatgtacaaccagaagttcaaggacaaggccacatta
actgtagacaagtcatccaatacagcctacatggagctcctcagtctgacttctgaggactctgcagtct
attactgtgcaagagataactacggtagtagtccagactttgactac
>24, SEQ ID NO: 486
gaagtgaagcttgtggagtctggacctgagctggtgaagcctgggacttcagtgaagatatcctgcaagg
cttctggctacaccttcactgactactatataaactgggtgaagcagaggcctggacagggacttgagtg
gattggttggatttatcctggaggcggtaatactaggtacattgagaggttcaagggcaaggccacattg
actgtagacacatcctccagcacagcctacatgcagctcagcagcctaacatctgaggacactgctgtct
atttctgtgcaagaaacggctactggtacttcgatgtc
>31, SEQ ID NO: 487
gaggtgcagcttaaggagtctggacctgagctgaagaagcctggagagacagtcaagatctcctgcatga
cttctgggtatatgttcacaaaccatggaatgaactgggtgaaacaggctccaggaaagggtttaaagtg
gatgggctggataaacacctacactggagagccaacatatggtgatggcttcaagggacggtttgtcttc
tctttggaaacctctgccagcactgcctatttgcagatcaacaacctcaaaaatgaggacacggctacat
atttctgtgcaagacgtgttgcgacgtacttcgatgtc
>11, SEQ ID NO: 488
gaggtgcagcttaaggagtctggggctgagctggtgaggcctgggggctcagtgaagatttcctgcaagg
gttctggctacacattcactaattttgctattcactgggtgaaacaaagtcatataaagactctagagtg
gattggtcttattagtacttcctctggtgatgttagctacaaccagaagttcaaggacaaggccacaatg
actgtagacaaatcctccagcactgcctatatggagcttgccagactgacatctgaggattctgccatct
attactgtgcaagacactatggtgccaacaactattttgactat
>32, SEQ ID NO: 489
gaggtgaagcttgttgagtctggacctgagttgaagaagcctggagagacagtcaagatctcctgcaagg
cttctgggtatatgttcacaaactatggaatgaattgggtgaagcaggctccaggaaagggtttaaagtg
gatgggctggataaacacctacactggagagccaacatatgttgaagacttcaagggacggtttgccttc
tctttggaaacctctgccaacactgcctatttgcagatcaacaacctcaaaaatgaggacacggctacat
atttttgtacacgaaggagtcatattaccttggactac
>12, SEQ ID NO: 490
gaggtgcagcttcaggagtctggggctgaactggtgaggcctggggtctcagtgaagatttcctgcaagg
gttctggctacacattcactaattttgctattcactgggtgaaacagagtcatgcaaagagtctagagtg
gattggacttattagttctaactctggtgatgttagctacaaccagaagttcaagggcaaggccacaatg
actgtagacaaatcctccagcacagcctatatggaacttgccagactgacatctgaggattctgccatct
attattgtgcaagacactatggtgcccacaactattttgactat
>14, SEQ ID NO: 491
gaggtgcagcttaaggagtctggggcagagcttgtgaagccaggggcctcagtcaagttgtcctgcacag
cttctggcttcaacattaaagacacctatatgcactgggtgaaacagaggcctgaacagggcctggagtg
gattggaaggattgatcctgcgaatggtaatactaaatatgacccgaagttccagggcaaggccactata
acagcagacacatcctccaacacagcctacctgcagctcagcagcctgacatctgaggacactgccgtct
attactgtgctagagaggagaattactacggtacctactactttgactac
>3, SEQ ID NO: 492
caggtgcagctgaaggagtctggacctgagctggtgaagcctggagcctcaatgaagatatcctgcaagg
cttcaggttattcattcaccggctacaccatgaactgggtgaagcagagccatggaaagaaccttgagtg
gattggacttattaatccttacaatggtggtactatgtacaaccagaagttcaagggcaaggccacatta
actgtagacaagtcatccaatacagcctacatggagctcctcagtctgacatctgaggactctgcagtct
attactgtgcaagagataactacggtagtagcccatactttgactac
>26, SEQ ID NO: 493
aaggtccagctgcaacagtctggacctgagctgaagaagcctggagagacagtcaagatctcctgcaagg
cttctgggtataccttcacaaactatggaatgaactgggtgaagcaggctccaggaaagggtttaaagtg
gatgggctggataaacacctacactggagagcctacatatgctgatgacttcaagggacggtttgccttc
tctttggaaacctctgccagaactgcctatttgcagatcaacaacctcaaaaatgaggactcggctacat
atttctgtgcaagaaggcgggacggaaactttgactac
>25, SEQ ID NO: 494
gaagtgaagcttgttgagtctggacctgagctggtgaagcctggggcttcagtgaggatatcttgcaagt
cctctggcaacaccttcacaaacttctatttacactggatgaaacagaggcctggacagggacttgagtg
gattggatgtatttatcctggaaacgttaagactaaatacagtgcgaggttcaagggcaaggccatactg
actgcggacaaatcctccagcacagtcttcatgcagctcagcaacctgacctctgaggactctgcggtct
atttctgtgcaaaggagggagattacgacgggacggcctactttgattac
>33, SEQ ID NO: 495
caggttactctgaaagagtctggacctgaactgaagaagcctggagagacagtcaagatctcctgcaggg
cttctgggtatatcttcacaaactatggaatgaactgggtgaagcaggctccaggaaagggtttaaagtg
gatgggctggataaacacctacactggagagccaacatatgctgatgacttcaagggacgatttgccttc
tctttggaaacctctgccagcactgcccatttgcagatcaacaacctcaaaaatgaggacacggctatat
atttctgtgcaagacggaggactacggcctttgactac
>5, SEQ ID NO: 496
gaagtgaagcttgtggagtctggacctgagctggtgaagcctgaagcttcagtgaagatatcctgcaagg
cttctggttactcattcactggctacaccatgaactgggtgaagcagagccatggaaagaaccttgaatg
gattggacttattaatccttacaatggtggtactacctacaaccagaagttcaagggcaaggccacatta
actgtagacacgtcatccagcacagccttcatggagctcctcagtctgacatctgaagactctgcagtct
attactgtgcaagagattactacggtagtagtccagactttgactac
>34, SEQ ID NO: 497
gaggtgaagctggtggagtctggggcagagcttgtgaggtcaggggcctcagtcaaattgtcctgcgcag
cttctggcttcaacattaaagactactatatacactgggtaaaacagaggcctgaacagggcctggagtg
gattggatggattgatcctgagaatggtcgtactgaatatgccccgaagttccagggcaaggccactatg
actgcagacacatcctccaacacagcctacctgcagctcagcagcctgacatctgaggacactgccgtct
attactgtaataatggtaactacgtcagacactactactttgactac
>35, SEQ ID NO: 498
caggtccagctgcaacagcctggacctgagctgaagaagcctggagagacagtcaagatctcctgcaagg
cttctgggtataccttcataaattatggaatgaactgggtgaagcaggctccaggaaagggtttaaagtg
gatgggctggataaacacctacactggagagccaacatatgctgatgacttcaagggacggtttgccttc
tctttggaaacctctgccagcactgcctatttgcagatcaacaacctcgaacatgaggacatggctgtat
atttctgtacaagaagaagagaaataacctttgactac
>13, SEQ ID NO: 499
caggtccagctgcaacagtctggggctgaactggtgaggccaggggtctcagtgaagatttcctgcaagg
gttctggctacacattcactaattttgctattcactgggtgaaacagagtcatgcaaagagtctagagtg
gattggacttattagttctaactctggtgatgttagctacaaccagaagttcaagggcaaggccacaatg
actgtagacaaatcctccagcacagcctatatggaacttgccagactgacatctgaggattctgccatct
attattgtgcaagacactatggtgcccacaactattttgactat
>9, SEQ ID NO: 500
caggtgcagctgaaggagtctggacctgagctggagaagcctggcgcttcagtgaagatatcctgcaagg
cttctggttactcattcactgcctacagcatgaactgggtgaagcagaacaatggaatgagccttgagtg
gattggaagtattgatccttattatggtgatactaagtacgcccaaaagttcaagggcaaggccacattg
actgtggacaaagcctccagcacagcctacttgcagctcaagagcctgacatctgaagactctgcagtct
attactgtgcaagaaggatgattacgacgggagactggtacttcgatgtc
>36, SEQ ID NO: 501
caggtccaactgcaacagcctggagctgagctggcgaggcccggggcttcagtgatgctgtcctgcaagg
cttctggctacaccttcactgactactttataaactgggtgaagcagaggactggacagggccttgactg
gattggagagatttatcctggaagtagtaatacttactacaatgaaaagttcaagggcaaggccacactg
actgcagacgaatcctccagcacagcctacatgcggctcagcagcctgacatctgaggactctgcagtct
agttctgtgcaagatcggggatttcgccctttacttac
>37, SEQ ID NO: 502
caggtgcagcttaaggagtctggggctgacctggtgaagcctggggcctcagtgaagatgtcctgcaaga
cttctggctacatttttaccggttacaatatacactgggtcaaacagacgcctggacagggcctggtttg
gattggagctgtttatccaggaaatggtgatacttcctacaatcagaatttcaaagccaaggccacattg
actgcagacatctcctccaccacagcctacatgcagctcagcagcctgacatctgaggactctgcgatct
attactgtgcaaaatatgaccgggggtttgcttcc

TABLE 6
Nucleotide and amino acid sequences of the constant kappa (light chain) and
heavy chain domains.

| | | | | Amino acid |
|---|---|---|---|---|
| | | Genbank | Nucleotide sequence | sequence |
| Chain | Gene | no. | SEQ ID NO | SEQ ID NO |
| Human | IGKC*01 | J00241 | Gaactgtggctgcaccatctgtcttcatcttcccgccatct | TVAAPSVFIFPPSD |
| constant | | | gatgagcagttgaaatctggaactgcctctgttgtgtgcct | EQLKSGTASVVCL |
| kappa chain | | | gctgaataacttctatcccagagaggccaaagtacagt | LNNFYPREAKVQW |
| | | | ggaaggtggataacgccctccaatcgggtaactccca | KVDNALQSGNSQE |
| | | | ggagagtgtcacagagcaggacagcaaggacagca | SVTEQDSKDSTYS |
| | | | cctacagcctcagcagcaccctgacgctgagcaaagc | LSSTLTLSKADYEK |
| | | | agactacgagaaacacaaagtctacgcctgcgaagtc | HKVYACEVTHQGL |
| | | | acccatcagggcctgagctcgcccgtcacaaagagctt | SSPVTKSFNRGEC |
| | | | caacaggggagagtgt | SEQ ID NO: 504 |
| | | | SEQ ID NO: 503 | |
| Human | IGHG1*03 | Y14737 | Tccaccaagggcccatcggtcttccccctggcaccctc | STKGPSVFPLAPS |
| constant | | | ctccaagagcacctctgggggcacagcggccctgggc | SKSTSGGTAALGC |
| heavy chain | | | tgcctggtcaaggactacttccccgaaccggtgacggtg | LVKDYFPEPVTVS |
| | | | tcgtggaactcaggcgccctgaccagcggcgtgcaca | WNSGALTSGVHTF |
| | | | ccttcccggctgtcctacagtcctcaggactctactccctc | PAVLQSSGLYSLS |
| | | | agcagcgtggtgaccgtgccctccagcagcttgggcac | SVVTVPSSSLGTQ |
| | | | ccagacctacatctgcaacgtgaatcacaagcccagc | TYICNVNHKPSNTK |
| | | | aacaccaaggtggacaagagagttgagcccaaatctt | VDKRVEPKSCDKT |
| | | | gtgacaaaactcacacatgcccaccgtgcccagcacc | HTCPPCPAPELLG |
| | | | tgaactcctggggggaccgtcagtcttcctcttcccccca | GPSVFLFPPKPKD |
| | | | aaacccaaggacaccctcatgatctcccggacccctg | TLMISRTPEVTCVV |
| | | | aggtcacatgcgtggtggtggacgtgagccacgaaga | VDVSHEDPEVKFN |
| | | | ccctgaggtcaagttcaactggtacgtggacggcgtgg | WYVDGVEVHNAKT |
| | | | aggtgcataatgccaagacaaagccgcgggaggagc | KPREEQYNSTYRV |
| | | | agtacaacagcacgtaccgtgtggtcagcgtcctcacc | VSVLTVLHQDWLN |
| | | | gtcctgcaccaggactggctgaatggcaaggagtaca | GKEYKCKVSNKAL |
| | | | agtgcaaggtctccaacaaagccctcccagcccccatc | PAPIEKTISKAKGQ |
| | | | gagaaaaccatctccaaagccaaagggcagccccga | PREPQVYTLPPSR |
| | | | gaaccacaggtgtacaccctgcccccatcccgggagg | EEMTKNQVSLTCL |
| | | | agatgaccaagaaccaggtcagcctgacctgcctggtc | VKGFYPSDIAVEW |
| | | | aaaggcttctatcccagcgacatcgccgtggagtggga | ESNGQPENNYKTT |
| | | | gagcaatgggcagccggagaacaactacaagacca | PPVLDSDGSFFLY |
| | | | cgcctcccgtgctggactccgacggctccttcttcctctat | SKLTVDKSRWQQ |
| | | | agcaagctcaccgtggacaagagcaggtggcagcag | GNVFSCSVMHEAL |
| | | | gggaacgtcttctcatgctccgtgatgcatgaggctctgc | HNHYTQKSLSLSP |
| | | | acaaccactacacgcagaagagcctctccctgtccccg | GK |
| | | | ggtaaatga | SEQ ID NO: 506 |
| | | | SEQ ID NO: 505 | |

TABLE 7
Amino acid sequence of human CD5.

SEQ ID NO: 507
>gi|7656965|ref|NP_055022.1|CD5 molecule [Homo
sapiens]
MPMGSLQPLATLYLLGMLVASCLGRLSWYDPDFQARLTRSNSKCQGQLEV
YLKDGWHMVCSQSWGRSSKQWEDPSQASKVCQRLNCGVPLSLGPFLVTYT
PQSSIICYGQLGSFSNCSHSRNDMCHSLGLTCLEPQKTTPPTTRPPPTTT
PEPTAPPRLQLVAQSGGQHCAGVVEFYSGSLGGTISYEAQDKTQDLENFL
CNNLQCGSFLKHLPETEAGRAQDPGEPREHQPLPIQWKIQNSSCTSLEHC
FRKIKPQKSGRVLALLCSGFQPKVQSRLVGGSSICEGTVEVRQGAQWAAL
CDSSSARSSLRWEEVCREQQCGSVNSYRVLDAGDPTSRGLFCPHQKLSQC
HELWERNSYCKKVFVTCQDPNPAGLAAGTVASIILALVLLVVLLVVCGPL
AYKKLVKKFRQKKQRQWIGPTGMNQNMSFHRNHTATVRSHAENPTASHVD
NEYSQPPRNSRLSAYPALEGVLHRSSMQPDNSSDSDYDLHGAQRL

TABLE 8
Nucleotide sequence of human CD5.

SEQ ID NO: 508
>gi|166197667|ref|NM_014207.3|Homo sapiens CD5
molecule (CD5), mRNA
ACGCCACCCCGCCCTCTCCCTCTCTGAGAGCGAGATACCCGGCCAGACAC
CCTCACCTGCGGTGCCCAGCTGCCCAGGCTGAGGCAAGAGAAGGCCAGAA
ACCATGCCCATGGGGTCTCTGCAACCGCTGGCCACCTTGTACCTGCTGGG
GATGCTGGTCGCTTCCTGCCTCGGACGGCTCAGCTGGTATGACCCAGATT
TCCAGGCAAGGCTCACCCGTTCCAACTCGAAGTGCCAGGGCCAGCTGGAG
GTCTACCTCAAGGACGGATGGCACATGGTTTGCAGCCAGAGCTGGGGCCG
GAGCTCCAAGCAGTGGGAGGACCCCAGTCAAGCGTCAAAAGTCTGCCAGC
GGCTGAACTGTGGGGTGCCCTTAAGCCTTGGCCCCTTCCTTGTCACCTAC
ACACCTCAGAGCTCAATCATCTGCTACGGACAACTGGGCTCCTTCTCCAA
CTGCAGCCACAGCAGAAATGACATGTGTCACTCTCTGGGCCTGACCTGCT
TAGAACCCCAGAAGACAACACCTCCAACGACAAGGCCCCCGCCCACCACA
ACTCCAGAGCCCACAGCTCCTCCCAGGCTGCAGCTGGTGGCACAGTCTGG
CGGCCAGCACTGTGCCGGCGTGGTGGAGTTCTACAGCGGCAGCCTGGGGG
GTACCATCAGCTATGAGGCCCAGGACAAGACCCAGGACCTGGAGAACTTC
CTCTGCAACAACCTCCAGTGTGGCTCCTTCTTGAAGCATCTGCCAGAGAC
TGAGGCAGGCAGAGCCCAAGACCCAGGGGAGCCACGGGAACACCAGCCCT
TGCCAATCCAATGGAAGATCCAGAACTCAAGCTGTACCTCCCTGGAGCAT
TGCTTCAGGAAAATCAAGCCCCAGAAAAGTGGCCGAGTTCTTGCCCTCCT
TTGCTCAGGTTTCCAGCCCAAGGTGCAGAGCCGTCTGGTGGGGGGCAGCA
GCATCTGTGAAGGCACCGTGGAGGTGCGCCAGGGGGCTCAGTGGGCAGCC
CTGTGTGACAGCTCTTCAGCCAGGAGCTCGCTGCGGTGGGAGGAGGTGTG
CCGGGAGCAGCAGTGTGGCAGCGTCAACTCCTATCGAGTGCTGGACGCTG
GTGACCCAACATCCCGGGGGCTCTTCTGTCCCCATCAGAAGCTGTCCCAG
TGCCACGAACTTTGGGAGAGAAATTCCTACTGCAAGAAGGTGTTTGTCAC
ATGCCAGGATCCAAACCCCGCAGGCCTGGCCGCAGGCACGGTGGCAAGCA
TCATCCTGGCCCTGGTGCTCCTGGTGGTGCTGCTGGTCGTGTGCGGCCCC
CTTGCCTACAAGAAGCTAGTGAAGAAATTCCGCCAGAAGAAGCAGCGCCA
GTGGATTGGCCCAACGGGAATGAACCAAAACATGTCTTTCCATCGCAACC
ACACGGCAACCGTCCGATCCCATGCTGAGAACCCCACAGCCTCCCACGTG
GATAACGAATACAGCCAACCTCCCAGGAACTCCCACCTGTCAGCTTATCC
AGCTCTGGAAGGGGCTCTGCATCGCTCCTCCATGCAGCCTGACAACTCCT
CCGACAGTGACTATGATCTGCATGGGGCTCAGAGGCTGTAAAGAACTGGG
ATCCATGAGCAAAAAGCCGAGAGCCAGACCTGTTTGTCCTGAGAAAACTG
TCCGCTCTTCACTTGAAATCATGTCCCTATTTCTACCCCGGCCAGAACAT
GGACAGAGGCCAGAAGCCTTCCGGACAGGCGCTGCTGCCCCGAGTGGCAG
GCCAGCTCACACTCTGCTGCACAACAGCTCGGCCGCCCCTCCACTTGTGG
AAGCTGTGGTGGGCAGAGCCCCAAAACAAGCAGCCTTCCAACTAGAGACT
CGGGGGTGTCTGAAGGGGGCCCCCTTTCCCTGCCCGCTGGGGAGCGGCGT
CTCAGTGAAATCGGCTTTCTCCTCAGACTCTGTCCCTGGTAAGGAGTGAC
AAGGAAGCTCACAGCTGGGCGAGTGCATTTTGAATAGTTTTTTGTAAGTA
GTGCTTTTCCTCCTTCCTGACAAATCGAGCGCTTTGGCCTCTTCTGTGCA
GCATCCACCCCTGCGGATCCCTCTGGGGAGGACAGGAAGGGGACTCCCGG
AGACCTCTGCAGCCGTGGTGGTCAGAGGCTGCTCACCTGAGCACAAAGAC
AGCTCTGCACATTCACCGCAGCTGCCAGCCAGGGGTCTGGGTGGGCACCA
CCCTGACCCACAGCGTCACCCCACTCCCTCTGTCTTATGACTCCCCTCCC
CAACCCCCTCATCTAAAGACACCTTCCTTTCCACTGGCTGTCAAGCCCAC
AGGGCACCAGTGCCACCCAGGGCCCGGCACAAAGGGGCGCCTAGTAAACC
TTAACCAACTTGGTTTTTTGCTTCACCCAGCAATTAAAAGTCCCAAGCTG
AGGTAGTTTCAGTCCATCACAGTTCATCTTCTAACCCAAGAGTCAGAGAT
GGGGCTGGTCATGTTCCTTTGGTTTGAATAACTCCCTTGACGAAAACAGA
CTCCTCTAGTACTTGGAGATCTTGGACGTACACCTAATCCCATGGGGCCT
CGGCTTCCTTAACTGCAAGTGAGAAGAGGAGGTCTACCCAGGAGCCTCGG
GTCTGATCAAGGGAGAGGCCAGGCGCAGCTCACTGCGGCGGCTCCCTAAG
AAGGTGAAGCAACATGGGAACACATCCTAAGACAGGTCCTTTCTCCACGC
CATTTGATGCTGTATCTCCTGGGAGCACAGGCATCAATGGTCCAAGCCGC
ATAATAAGTCTGGAAGAGCAAAAGGGAGTTACTAGGATATGGGGTGGGCT
GCTCCCAGAATCTGCTCAGCTTTCTGCCCCCACCAACACCCTCCAACCAG
GCCTTGCCTTCTGAGAGCCCCCGTGGCCAAGCCCAGGTCACAGATCTTCC
CCCGACCATGCTGGGAATCCAGAAACAGGGACCCCATTTGTCTTCCCATA
TCTGGTGGAGGTGAGGGGGCTCCTCAAAAGGGAACTGAGAGGCTGCTCTT
AGGGAGGGCAAAGGTTCGGGGGCAGCCAGTGTCTCCCATCAGTGCCTTTT
TTAATAAAAGCTCTTTCATCTATAGTTTGGCCACCATACAGTGGCCTCAA
AGCAACCATGGCCTACTTAAAAACCAAACCAAAAATAAAGAGTTTAGTTG
AGGAGAAAAAAAAAAAAAAAAAAAAAAAAA

TABLE 13
Affinity of anti-CD5 antibodies.

| Clone no. | ka (1/Ms) | kd (1/s) | KD (M) |
|---|---|---|---|
| 1 | 1.07E+05 | 5.57E−04 | 5.19E−09 |
| 8 | 2.65E+04 | 9.81E−06 | 3.70E−10 |
| 9 | 1.42E+04 | 6.17E−05 | 4.35E−09 |
| 11 | 6.99E+04 | 2.20E−05 | 3.15E−10 |
| 12 | 9.70E+04 | 1.15E−04 | 1.18E−09 |
| 14 | 3.21E+05 | 1.80E−03 | 5.60E−09 |
| 15 | 5.44E+04 | 1.27E−03 | 2.34E−08 |
| 17 | 5.15E+05 | 8.45E−05 | 1.64E−10 |
| 18 | 1.60E+05 | 3.27E−05 | 2.05E−10 |
| 21 | 2.49E+03 | 3.15E−04 | 4.21E−08 |
| 23 | 2.19E+04 | 1.05E−04 | 4.79E−09 |
| 29 | 6.55E+05 | 1.37E−03 | 2.09E−09 |
| 31 | 9.07E+04 | 8.03E−04 | 8.85E−09 |
| 32 | 1.28E+05 | 1.16E−04 | 9.03E−10 |
| 34 | 1.37E+04 | 8.40E−04 | 6.13E−08 |

Epitope Mapping

The CD5 molecule is a transmembrane glycoprotein consisting of a cytoplasmic (intracellular) domain, a transmembrane domain and three extracellular domains (I, II, and III). Extracellular domain I, being the most amino-terminal domain and furthest from the cell membrane, is usually considered to be the most immunogenic domain of CD5. A number of monoclonal antibodies have been raised against domain I of CD5. However, domains II and III of CD5 are more conserved among mammalian species than domain I. Thus, antibodies binding the more conserved epitopes on domains II or III are generally preferable in order to avoid lack of binding due to a mutated epitope. Also, compositions comprising antibodies recognizing distinct epitopes on different domains are preferable. This is so for at least two reasons. Firstly, a composition comprising antibodies recognizing distinct epitopes on different domains is less sensitive to mutation of an epitope. Secondly, it may be desirable to achieve simultaneous binding of a plurality of antibodies to the CD5 molecule. This is more likely to happen with antibodies recognizing epitopes on different domains.

Uses of the Antibody Compositions of the Invention

The compositions of the invention can be used for in vivo treatment and prevention of diseases related to cells expressing CD5. The compositions of the invention are administered to patients (e. g., human subjects) at therapeutically effective dosages (e. g., dosages which result in growth inhibition, phagocytosis, reduction of motility, terminal differentiation, and/or killing of cells expressing CD5) using any suitable route of administration, such as injection and other routes of administration known in the art for antibody-based clinical products.

Diseases, which involve cells expressing CD5, and which can be treated, ameliorated, and/or prevented using the antibodies of the invention include, but are not limited to cancers, transplantations, autoimmune diseases and inflammatory diseases. Preferably, the disease to be treated by the compositions of the present invention is CLL. The compositions of the present invention may also be used in relation to treatment, amelioration or prevention of rheumatoid arthritis. Furthermore, the compositions of the invention may be used in relation to treatment, amelioration or prevention of acute T-cell leukaemia, cutaneous t-cell lymphoma, and diffuse large B-cell lymphoma.

Production of Antibodies of the Invention

An antibody composition of the present invention may be produced from a polyclonal expression cell line in one or a few bioreactors or equivalents thereof. Following this approach the anti-CD5 antibodies can be purified from the reactor as a single preparation without having to separate the individual members constituting the anti-CD5 antibody composition during the process. If the antibody composition is produced in more than one bioreactor, the purified anti-CD5 antibody composition can be obtained by pooling the antibodies obtained from individually purified supernatants from each bioreactor.

One way of producing a recombinant antibody composition is described in WO 2004/061104 and WO 2006/007850 (these references are hereby incorporated by reference). The method described therein, is based on site-specific integration of the antibody coding sequence into the genome of the individual host cells, ensuring that the VH and VL protein chains are maintained in their original pairing during production. Furthermore, the site-specific integration minimises position effects and therefore the growth and expression properties of the individual cells in the polyclonal cell line are expected to be very similar. Generally, the method involves the following: i) a host cell with one or more recombinase recognition sites; ii) an expression vector with at least one recombinase recognition site compatible with that of the host cell; iii) generation of a collection of expression vectors by transferring the selected VH and VL coding pairs from the screening vector to an expression vector such that a full-length antibody or antibody fragment can be expressed from the vector (such a transfer may not be necessary if the screening vector is identical to the expression vector); iv) transfection of the host cell with the collection of expression vectors and a vector coding for a recombinase capable of combining the recombinase recognition sites in the genome of the host cell with that in the vector; v) obtaining/generating a polyclonal cell line from the transfected host cell and vi) expressing and collecting the antibody composition from the polyclonal cell line.

When a small number (2-3 or more) of antibodies are used for one composition these may be expressed and purified individually in a way similar to manufacture of monoclonal antibodies, for example as described in WO 2004/085474. The purified antibodies can be mixed after purification or be packaged in separate vials for mixing prior to administration or for separate administration.

Preferably mammalian cells such as CHO cells, COS cells, BHK cells, myeloma cells (e.g., Sp2/0 or NS0 cells), fibroblasts such as NIH 3T3, and immortalized human cells, such as HeLa cells, HEK 293 cells, or PER.C6, are used. However, non-mammalian eukaryotic or prokaryotic cells, such as plant cells, insect cells, yeast cells, fungi, E. coli etc., can also be employed. A suitable host cell comprises one or more suitable recombinase recognition sites in its genome. The host cell should also contain a mode of selection which is operably linked to the integration site, in order to be able to select for integrants, (i.e., cells having an integrated copy of an anti-CD5 Ab expression vector or expression vector fragment in the integration site). The preparation of cells having an FRT site at a pre-determined location in the genome was described in e.g. U.S. Pat. No. 5,677,177. Preferably, a host cell only has a single integration site, which is located at a site allowing for high expression of the integrant (a so-called hot-spot).

A suitable expression vector comprises a recombination recognition site matching the recombinase recognition site(s) of the host cell. Preferably the recombinase recognition site is linked to a suitable selection gene different from the selection gene used for construction of the host cell. Selection genes are well known in the art, and include glutamine synthetase gene (GS), dihydrofolate reductase gene (DHFR), and neomycin, where GS or DHFR may be used for gene amplification of the inserted VH and VL sequence. The vector may also contain two different recombinase recognition sites to allow for recombinase-mediated cassette exchange (RMCE) of the antibody coding sequence instead of complete integration of the vector. RMCE is described in (Langer et al 2002; Schlake and Bode 1994). Suitable recombinase recognition sites are well known in the art, and include FRT, lox and attP/attB sites. Preferably the integrating vector is an isotype-encoding vector, where the constant regions (preferably including introns) are present in the vector prior to transfer of the VH and VL coding pair from the screening vector (or the constant regions are already present in the screening vector if screening is performed on full-length antibodies). The constant regions present in the vector can either be the entire heavy chain constant region (CH1 to CH3 or to CH4) or the constant region encoding the Fc part of the antibody (CH2 to CH3 or to CH4). The light chain Kappa or Lambda constant region may also be present prior to transfer. The choice of the number of constant regions present, if any, depends on the screening and transfer system used. The heavy chain constant regions can be selected from the isotypes IgG1, IgG2, IgG3, IgG4, IgA1, IgA2, IgM, IgD and IgE. Preferred isotypes are IgG1, IgG2, and/or IgG3. Further, the expression vector for site-specific integration of the anti-CD5 antibody-encoding nucleic acid contains suitable promoters or equivalent sequences directing high levels of expression of each of the VH and VL chains.

The transfer of the selected VH and VL coding pairs from the screening vector can be performed by conventional restriction enzyme cleavage and ligation, such that each expression vector molecule contain one VH and VL coding pair. Preferably, the VH and VL coding pairs are transferred individually, they may, however, also be transferred in-mass if desired. When all the selected VH and VL coding pairs are transferred to the expression vector a collection or a library of expression vectors is obtained. Alternative ways of transfer may also be used if desired. If the screening vector is identical to the expression vector, the library of expression vectors is constituted of the VH and VL sequence pairs selected during screening, which are situated in the screening/expression vector.

Methods for transfecting a nucleic acid sequence into a host cell are known in the art. To ensure site-specific integration, a suitable recombinase must be provided to the host cell as well. This is preferably accomplished by co-transfection of a plasmid encoding the recombinase. Suitable recombinases are for example Flp, Cre or phage φC31 integrase, used together with a host cell/vector system with the corresponding recombinase recognition sites. The host cell can either be transfected in bulk, meaning that the library of expression vectors is transfected into the cell line in one single reaction thereby obtaining a polyclonal cell line. Alternatively, the collection of expression vectors can be transfected individually into the host cell, thereby generating a collection of individual cell lines (each cell line produce an antibody with a particular specificity). The cell lines generated upon transfection (individual or polyclonal) are then selected for site specific integrants, and adapted to grow in suspension and serum free media, if they did not already have these properties prior to transfection. If the transfection was performed individually, the individual cell lines are analyzed further with respect to their grow properties and antibody production. Preferably, cell lines with similar proliferation rates and antibody expression levels are selected for the generation of the polyclonal cell line. The polyclonal cell line is then generated by mixing the individual cell lines in a predefined ratio. Generally, a polyclonal master cell bank (pMCB), a polyclonal research cell bank (pRCB) and/or a polyclonal working cell bank (pWCB) are laid down from the polyclonal cell line. The polyclonal cell line is generated by mixing the individual cell lines in a predefined ratio. The polyclonal cell line is distributed into ampoules thereby generating a polyclonal research cell bank (pRCB) or master cell bank (pMCB) from which a polyclonal working cell bank (pWCB) can be generated by expanding cells from the research or master cell bank. The research cell bank is primarily for proof of concept studies, in which the polyclonal cell line may not comprise as many individual antibodies as the polyclonal cell line in the master cell bank. Normally, the pMCB is expanded further to lay down a pWCB for production purposes. Once the pWCB is exhausted a new ampoule from the pMCB can be expanded to lay down a new pWCB.

One embodiment of the present invention is a polyclonal cell line capable of expressing a recombinant anti-CD5 antibody composition of the present invention.

A further embodiment of the present invention is a polyclonal cell line wherein each individual cell is capable of expressing a single VH and VL coding pair, and the polyclonal cell line as a whole is capable of expressing a collection of VH and VL encoding pairs, where each VH and VL pair encodes an anti-CD5 antibody. Preferably the collection of VH and VL coding pairs are cognate pairs generated according to the methods of the present invention.

A recombinant antibody composition of the present invention may be manufactured by culturing one ampoule from a pWCB in an appropriate medium for a period of time allowing for sufficient expression of antibody and where the polyclonal cell line remains stable (The window is approximately between 15 days and 50 days). Culturing methods such as fed batch or perfusion may be used. The recombinant antibody composition is obtained from the culture medium and purified by conventional purification techniques. Affinity chromatography combined with subsequent purification steps such as ion-exchange chromatography, hydrophobic interactions and gel filtration has frequently been used for the purification of IgG. Following purification, the presence of all the individual members in the polyclonal antibody composition is assessed, for example by ion-exchange chromatography. The characterization of such an antibody composition is described in detail in WO 2006/007853 (hereby incorporated by reference).

An alternative method of expressing a mixture of antibodies in a recombinant host is described in WO 2004/009618. This method produces antibodies with different heavy chains associated with the same light chain from a single cell line. This approach may be applicable if the anti-CD5 antibody composition is produced from a combinatorial library.

Therapeutic Compositions

Another aspect of the invention is a pharmaceutical composition comprising as an active ingredient an anti-CD5 antibody composition or anti-CD5 recombinant Fab or another anti-CD5 recombinant antibody fragment composition, or a bi-specific binding molecule of the invention. Preferably, the active ingredient of such a composition is an anti-CD5 recombinant antibody composition as described in the present invention. Such compositions are intended for amelioration and/or prevention and/or treatment of cancer, such as CLL. Also, such compositions may be intended for amelioration and/or prevention and/or treatment of rheumatoid arthritis. Preferably, the pharmaceutical composition is administered to a human, a domestic animal, or a pet.

The pharmaceutical composition may further comprise a pharmaceutically acceptable excipient.

Anti-CD5 antibody composition or fragments of the antibodies thereof may be administered within a pharmaceutically-acceptable diluent, carrier, or excipient, in unit dosage form. Conventional pharmaceutical practice may be employed to provide suitable formulations or compositions to administer to patients. In a preferred embodiment the administration is therapeutic, meaning that it is administered after a disease condition has been diagnosed. Any appropriate route of administration may be employed, for example, administration may be parenteral, intravenous, intra-arterial, subcutaneous, intramuscular, intraperitoneal, intranasal, aerosol, suppository, or oral administration. For example, pharmaceutical formulations may be in the form of liquid solutions or suspensions. For intranasal formulations, antibodies may be administered in the form of powders, nasal drops, or aerosols.

The pharmaceutical compositions of the present invention are prepared in a manner known per se, for example, by means of conventional dissolving, lyophilizing, mixing, granulating, or confectioning processes. The pharmaceutical compositions may be formulated according to conventional pharmaceutical practice (see for example, in Remington: The Science and Practice of Pharmacy (20th ed.), ed. A. R. Gennaro, 2000, Lippincott Williams & Wilkins, Philadelphia, Pa. and Encyclopedia of Pharmaceutical Technology, eds. J. Swarbrick and J. C. Boylan, 1988-1999, Marcel Dekker, New York, N.Y.).

Preferably solutions or suspensions of the active ingredient, and especially isotonic aqueous solutions or suspensions, are used to prepare pharmaceutical compositions of the present invention. In the case of lyophilized compositions that comprise the active ingredient alone or together with a carrier, for example mannitol, such solutions or suspensions may, if possible, be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The said solutions or suspensions may comprise viscosity-increasing substances, such as sodium carboxymethylcellulose, carboxymethylcellulose, dextran, polyvinylpyrrolidone or gelatin.

The injection compositions are prepared in customary manner under sterile conditions; the same applies also to introducing the compositions into ampoules or vials and sealing of the containers.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, tablets, pills, or capsules. The formulations can be administered to human individuals in therapeutically or prophylactically effective amounts (e.g., amounts which prevent, eliminate, or reduce a pathological condition) to provide therapy for a disease or condition. The preferred dosage of therapeutic agent to be administered is likely to depend on such variables as the severity of the disease, the overall health status of the particular patient, the formulation of the compound excipients, and its route of administration.

Diagnostic Use

Another embodiment of the invention is directed to diagnostic kits. Kits according to the present invention comprise an anti-CD5 antibody composition prepared according to the invention which protein may be labelled with a detectable label or non-labelled for non-label detection. The kit may be used to identify individuals inflicted with cancer associated with overexpression of CD5.

Antibody Compositions of the Invention

In an aspect, the present invention relates to an antibody composition comprising at least three, such as 3 or at least 4, such as 4 or at least 5, such as 5 or at least 6, such as 6 anti-CD5 antibodies capable of binding distinct domain I epitopes. In an aspect, said distinct epitopes are non-overlapping epitopes of domain I.

In an aspect, the present invention relates to an antibody composition comprising at least three, such as 3 or at least 4, such as 4 or at least 5, such as 5 or at least 6, such as 6 anti-CD5 antibodies capable of binding distinct epitopes. In an aspect, said distinct epitopes are non-overlapping epitopes.

In an aspect, the present invention relates to an antibody composition selected from the following compositions of anti-CD5 antibodies binding distinct epitopes:

| Compositions | | |
|---|---|---|
| with two | Compositions with | Compositions with four |
| antibodies | three antibodies | antibodies |
| Ab9 + Ab18 | Ab9 + Ab18 + Ab15 | Ab9 + Ab18 + Ab15 + Ab31 |
| Ab9 + Ab15 | Ab9 + Ab18 + Ab31 | Ab9 + Ab18 + Ab15 + Ab14 |
| Ab9 + Ab31 | Ab9 + Ab18 + Ab14 | Ab9 + Ab18 + Ab15 + Ab17 |
| Ab9 + Ab14 | Ab9 + Ab18 + Ab17 | Ab9 + Ab18 + Ab31 + Ab14 |
| Ab9 + Ab17 | Ab9 + Ab15 + Ab31 | Ab9 + Ab18 + Ab31 + Ab17 |
| Ab18 + Ab15 | Ab9 + Ab15 + Ab14 | Ab9 + Ab18 + Ab14 + Ab17 |
| Ab18 + Ab31 | Ab9 + Ab15 + Ab17 | Ab9 + Ab15 + Ab31 + Ab14 |
| Ab18 + Ab14 | Ab9 + Ab31 + Ab14 | Ab9 + Ab15 + Ab31 + Ab17 |
| Ab18 + Ab17 | Ab9 + Ab31 + Ab17 | Ab9 + Ab15 + Ab14 + Ab17 |
| Ab15 + Ab31 | Ab9 + Ab14 + Ab17 | Ab9 + Ab31 + Ab14 + Ab17 |
| Ab15 + Ab14 | Ab18 + Ab15 + Ab31 | Ab18 + Ab15 + Ab31 + Ab14 |
| Ab15 + Ab17 | Ab18 + Ab15 + Ab14 | Ab18 + Ab15 + Ab31 + Ab17 |
| Ab31 + Ab14 | Ab18 + Ab15 + Ab17 | Ab18 + Ab15 + Ab14 + Ab17 |
| Ab31 + Ab17 | Ab18 + Ab31 + Ab14 | Ab18 + Ab31 + Ab14 + Ab17 |
| Ab14 + Ab17 | Ab18 + Ab31 + Ab17 | Ab15 + Ab31 + Ab14 + Ab17 |
| | Ab18 + Ab14 + Ab17 | |
| | Ab15 + Ab31 + Ab14 | |
| | Ab15 + Ab31 + Ab17 | |
| | Ab15 + Ab14 + Ab17 | |
| | Ab31 + Ab14 + Ab17 | |

| Compositions with | Compositions with |
|---|---|
| five antibodies | six antibodies |
| Ab9 + Ab18 + Ab15 + Ab31 + Ab14 | Ab9 + Ab18 + Ab15 + Ab31 + |
| Ab9 + Ab18 + Ab15 + Ab31 + Ab17 | Ab14 + Ab17 |
| Ab9 + Ab18 + Ab15 + Ab14 + Ab17 | |
| Ab9 + Ab18 + Ab31 + Ab14 + Ab17 | |
| Ab9 + Ab15 + Ab31 + Ab14 + Ab17 | |
| Ab18 + Ab15 + Ab31 + Ab14 + Ab17 | |

Compositions of the Invention Comprising High CD5-Affinity Antibodies

In an aspect, the anti-CD5 antibodies of the antibody compositions of the present invention are selected for their CD5 affinity according to table 13 herein. In an aspect, the antibody compositions comprise antibodies with relatively high affinity towards CD5.

Antibody Compositions of the Invention and CD5 Internalization

In an aspect, the antibody composition of the present invention is capable of causing internalization of CD5. Internalization of CD5 may lead to degradation of CD5. Internalization of CD5 can effectively block the signal pathway downstream from CD5 and thereby reduce CD5 signalling. Thus CD5 functions can effectively be blocked by targeting CD5 with an antibody composition of the present invention, wherein said antibody composition is capable of causing CD5 internalization, optionally followed by intracellular degradation of CD5.

In CLL, the pathology is characterised by an accumulation of predominantly slowly dividing CD5-positive B lymphocytes. The accumulation is mostly caused by increased (pathological) survival of cells, rather than by excessive proliferation. The increased survival of the cells is at least partly due to failure to undergo programmed cell death (apoptosis). This same failure to undergo apoptosis lies behind the inherent resistance of CLL to chemotherapy. One way of overcoming the pathology of CLL is to cause clearance of the accumulated cells. Current therapy can involve removal of a part of the patients own blood, and thereby removal of some of the accumulated cells, and replacement with donor blood without accumulated cells. In relation to the accumulated cells, internalization and degradation of CD5 will most likely not lead to significant clearance of the cells. Thus, compositions of the present invention capable of causing internalization of CD5 are not preferred in this respect. However, there may be other positive effects associated with the internalization and degradation of CD5. In such cases, compositions causing internalization may be beneficial.

An antibody composition capable of causing CD5 internalization can be selected from anti-CD5 antibody compositions comprising the following antibody combinations: 9+14+15+17+18+31, 9+14+15+17+18, 9+15+18+31, 9+15+18.

An antibody composition capable of causing CD5 degradation can be selected from anti-CD5 antibody compositions comprising the following antibody combinations: 9+14+15+17+18+31 and 9+15+18.

Antibody Compositions of the Invention and Clearance of CD5-Positive Cells

In an aspect, binding to CD5 of the antibodies of the composition of the present invention does not lead to internalization of CD5. In this manner, CD5 bound by the said antibodies remains on the surface of the CD5-positive cell, thus allowing for clearance of the cell by e.g. the effector mechanisms ADCC and CDC. It may be advantageous to allow these effector mechanisms to take effect in order to get the CD5-positive cells cleared. Therefore, it may be advantageous to employ an antibody composition, wherein the antibodies of the composition remain on the surface of the CD5-positive cell after said antibodies have bound to CD5.

As discussed herein above, CLL is characterized by a pathological accumulation of cells. This accumulation may be remedied by a composition not capable of causing internalization of CD5 and thereby capable of leading to clearance of cells by e.g. the effector mechanisms ADCC and CDC. Thus, by employing a composition of the invention not leading to CD5 internalization, the CD5 positive cells can be specifically targeted by effector mechanisms such as ADCC and CDC and thus cleared from the system. This approach can thus counter the accumulation caused by the failure of the CD5 positive lymphocytes to undergo apoptosis. Thus, compositions of the present invention which are capable of causing clearance of CD5-positive B lymphocytes, such as compositions not causing internalization of CD5, are preferred.

An antibody composition, wherein binding to CD5 of the antibodies of the composition does not lead to internalization of CD5 can be selected from anti-CD5 antibody compositions comprising the following combinations of antibodies: 14+17, and 17+18.

An antibody composition not capable of causing CD5 degradation can comprises the antibodies 14+17.

### EXAMPLES

### Example 1

### Cloning of Anti-CD5 Antibodies

Immunizations

Female BALB/c, strain A (8-10 weeks old) were used for immunizations by injections with CD5-human growth hormone (hGH) fusion protein.

Inhouse made recombinant CD5-extracellular domain (ECD) was used for all immunizations. CD5-ECD was produced as a fusion protein consisting of the ECD of CD5 and human growth hormone (hGH), separated by a Tobacco Etch Virus (TEV)-cleavage site.

CD5-hGH was diluted in PBS and then mixed 1:1 with Freund's Adjuvant. Adjuvant is used to enhance and modulate the immune response. For the first immunizations Complete Freund's Adjuvant (CFA) was used whereas Incomplete Freund's Adjuvant (IFA) was used for the subsequent immunizations. IFA is an oil-in-water emulsion composed of mineral oils and CFA is IFA to which heat-killed, dried Mycobacterium species are added. Both adjuvants have a depot effect. CFA gives rise to long-term persistence of the immune response and is used for the first immunizations to boost the immune response and IFA is used for subsequent immunizations. The emulsions were tested by adding a drop on the surface of a glass with water. If the drop remains as one drop, the emulsion is stable and the injections can be performed. Only stable emulsions were administered to mice. 50 μg CD5-hGH was used for each injection. In total, mice received 4 injections. All mice were injected with 100 μl emulsion. Injections were performed subcutaneously (s.c.).

At termination, the mice were sacrificed Day 6 by injected of Hypnorm-Dormicum, and the spleens were removed and transferred to a 74 μm cell strainer (Corning#136350-3479). The cells were macerated through the filter, resuspended in cold RPMI 1640 with 10% FBS and centrifuged at 300×g for 5 minutes. The cell pellet was resuspended in RPMI 1640 with 1% FBS, filtered through a 50 μm syringe filter (BD#340603) and collected by centrifugation. The cell pellet was cryopreserved after resuspension in FCS with 10% DMSO and frozen cells stored at −80° C. until FACS sorting. FACS sorting of murine plasma cells

Vials with frozen splenocytes were thawed at 37° C. and transferred to 15 ml tube with ice still present. 10 ml Ice-cold RPMI, 10% FBS (foetal bovine serum) was drop-wise added to the tube while swirling. After one wash in 10 ml FACS PBS, 5 ml FCS PBS is added before filtering the cells through 50 μm Filcon. Cells were then pelleted and resuspended in 1 ml PBS with 2% FBS (final volume) and stained with anti-CD43-FITC and anti-CD138-PE according the specific dilution (app. 5 μg/ml.). Cells were incubated at 4° C. for 20 min in the dark. Subsequently, cells were washed 2 times with 2 ml FACS buffer. Up to 15 ml FACS PBS were added. Propidium Iodide (PI) was added 1:100, and cells were subsequently sorted into 96 well PCR-plates, containing PCR reaction buffer (see below), and spun down for 2 min 400×g before the plates were frozen at −80° C. Plasma cells were gated as CD43-positive/CD-138 positive as shown in FIG. 1.

Linkage of Cognate VH and VL Pairs

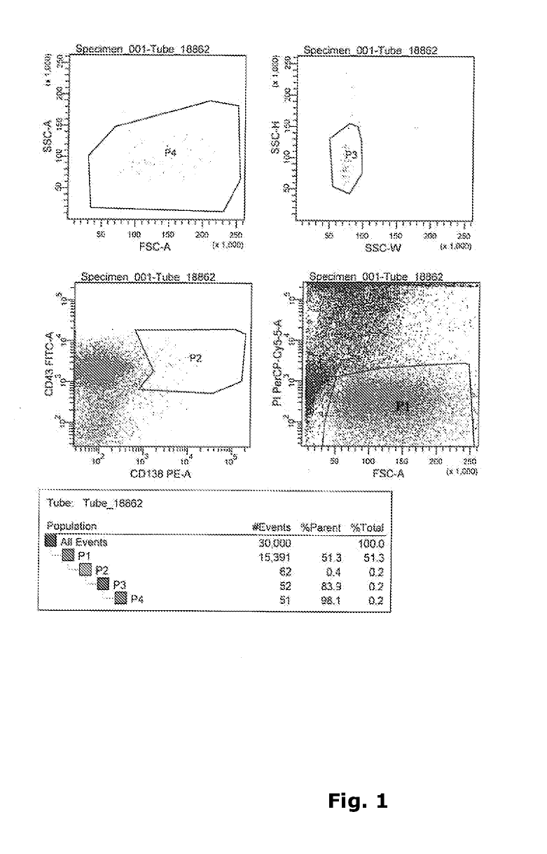

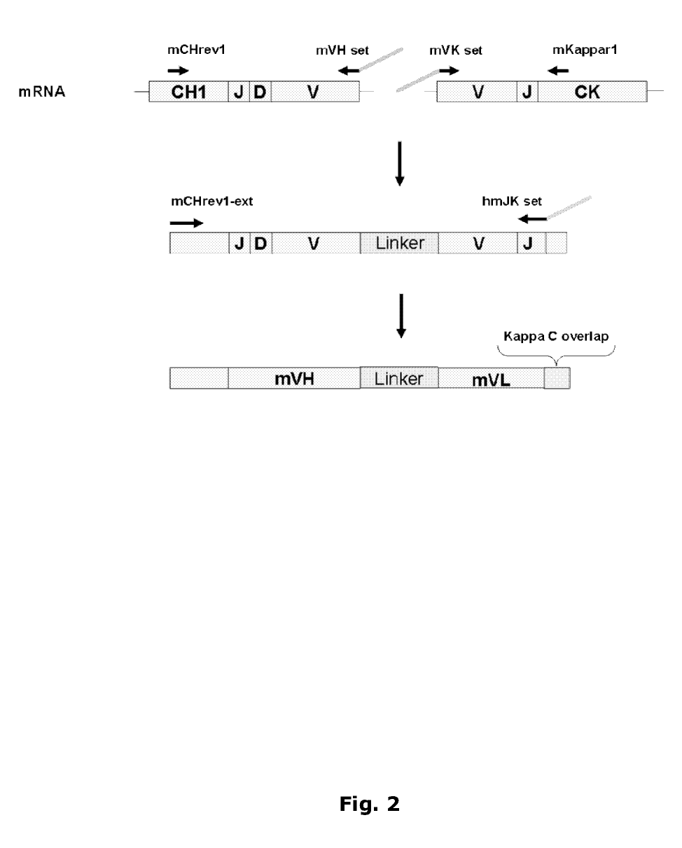

The linkage of VH and VL coding sequences was performed on the single cells gated as plasma cells, facilitating cognate pairing of the VH and VL coding sequences. The procedure utilized a two step PCR procedure based on a one-step multiplex overlap-extension RT-PCR followed by a nested PCR. The primer mixes used in the present example only amplify Kappa light chains. Primers capable of amplifying Lambda light chains could, however, be added to the multiplex primer mix and nested PCR primer mix if desired. If Lambda primers are added, the sorting procedure should be adapted such that Lambda positive cells are not excluded. The principle for linkage of cognate VH and VL sequences is illustrated in FIG. 2.

The 96-well PCR plates produced were thawed and the sorted cells served as template for the multiplex overlap-extension RT-PCR. The sorting buffer added to each well before the single-cell sorting contained reaction buffer (OneStep RT-PCR Buffer; Qiagen), primers for RT-PCR (see Table 10) and RNase inhibitor (RNasin, Promega). This was supplemented with OneStep RT-PCR Enzyme Mix (25× dilution; Qiagen) and dNTP mix (200 μM each) to obtain the given final concentration in a 20-μl reaction volume. The plates were incubated for 30 min at 55° C. to allow for reverse transcription of the RNA from each cell. Following the RT, the plates were subjected to the following PCR cycle: 10 min at 94° C., 35×(40 sec at 94° C., 40 sec at 60° C., 5 min at 72° C.), 10 min at 72° C.

The PCR reactions were performed in H20BIT Thermal cycler with a Peel Seal Basket for 24 96-well plates (ABgene) to facilitate a high-throughput. The PCR plates were stored at −20° C. after cycling.

For the nested PCR step, 96-well PCR plates were prepared with the following mixture in each well (20-μl reactions) to obtain the given final concentration: 1× FastStart buffer (Roche), dNTP mix (200 μM each), nested primer mix (see Table 11), Phusion DNA Polymerase (0.08 U; Finnzymes) and FastStart High Fidelity Enzyme Blend (0.8 U; Roche). As template for the nested PCR, 1 μl was transferred from the multiplex overlap-extension PCR reactions. The nested PCR plates were subjected to the following thermocyling: 35×(30 sec at 95° C., 30 sec at 60° C., 90 sec at 72° C.), 10 min at 72° C.

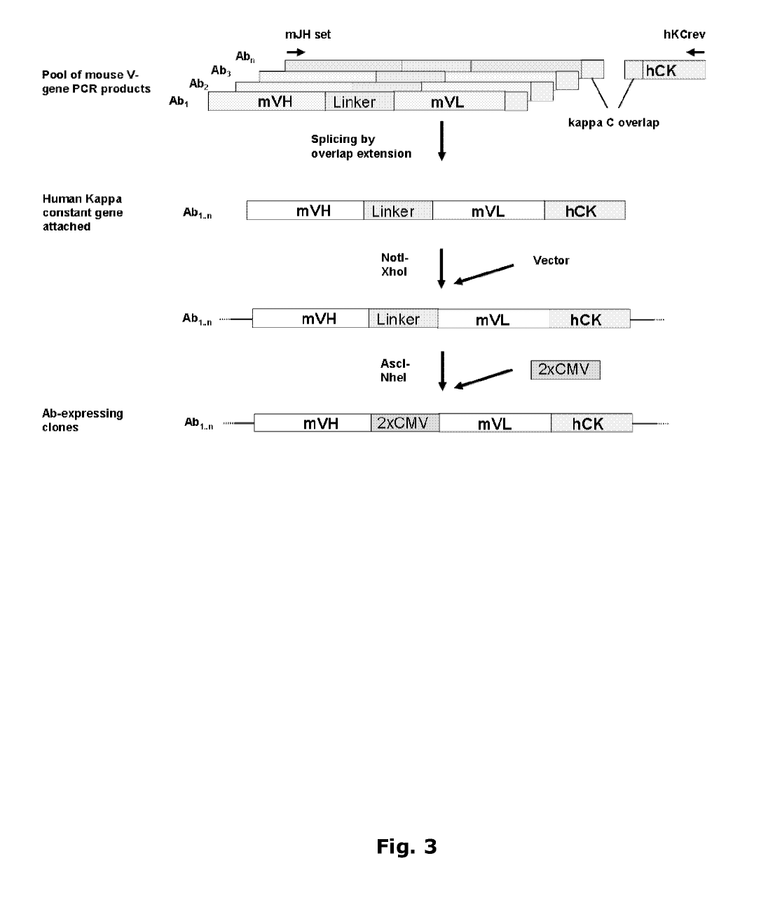

Randomly selected reactions were analyzed on a 1% agarose gel to verify the presence of an overlap-extension fragment of approximately 890 basepairs (bp). The plates were stored at −20° C. until further processing of the PCR fragments. The repertoires of linked VH and VL coding pairs from the nested PCR were pooled, without mixing pairs from different donors, and were purified by preparative 1% agarose gel electrophoresis. The human kappa constant light chain encoding sequence was spliced by overlap extension to the VL coding region of the pooled PCR products of linked VH and VL coding pairs (FIG. 3). The human kappa constant light chain encoding sequence was amplified from a plasmid containing the coding sequence of a human antibody with a kappa light chain in a reaction containing: Phusion Enzyme (2 U; Finnzymes), 1× Phusion buffer, dNTP mix (200 μM each), hKCforw-v2 primer and Kappa3′ primer (Table 12), and plasmid template pLL138 (10 ng/μl) in a total volume of 50 μl. The reaction was subjected to the following thermocycling: 25×(30 sec at 95° C., 30 sec at 55° C., 45 sec at 72° C.), 10 min at 72° C. The resulting PCR fragment was purified by preparative 1% agarose gel electrophoresis.

The purified pooled PCR fragments of each repertoire was spliced to the amplified and purified PCR fragment of the human kappa constant encoding region (Appendix 2) by the following splicing by overlap extension PCR (50 μl total volume) containing: human kappa constant encoding region fragment (1.4 ng/μl), purified pooled PCR fragment (1.4 ng/μl), Phusion DNA Polymerase (0.5 U; Finnzymes) and FastStart High Fidelity Enzyme Blend (0.2 U; Roche), 1× FastStart buffer (Roche), dNTP mix (200 μM each), mhKCrev primer and mJH set primers (see Table 12). The reaction was subjected to the following thermocycling: 2 min at 95° C., 25×(30 sec at 95° C., 30 sec at 55° C., 1 min at 72° C.), 10 min at 72° C. The resulting PCR fragment was purified by preparative 1% agarose gel electrophoresis.

Insertion of cognate VH and VL coding pairs into a screening vector In order to identify antibodies with binding specificity to CD5, the VH and VL coding sequences obtained were expressed as full-length antibodies. This involved insertion of the repertoire of VH and VL coding pairs into an expression vector and transformation into a host cell.

A two-step cloning procedure was employed for generation of a repertoire of expression vectors containing the linked VH and VL coding pairs. Statistically, if the repertoire of expression vectors contains ten times as many recombinant plasmids as the number of cognate paired VH and VL PCR products used for generation of the screening repertoire, there is 99% likelihood that all unique gene pairs are represented. Thus, if 400 overlap-extension V-gene fragments were obtained, a repertoire of at least 4000 clones was generated for screening.

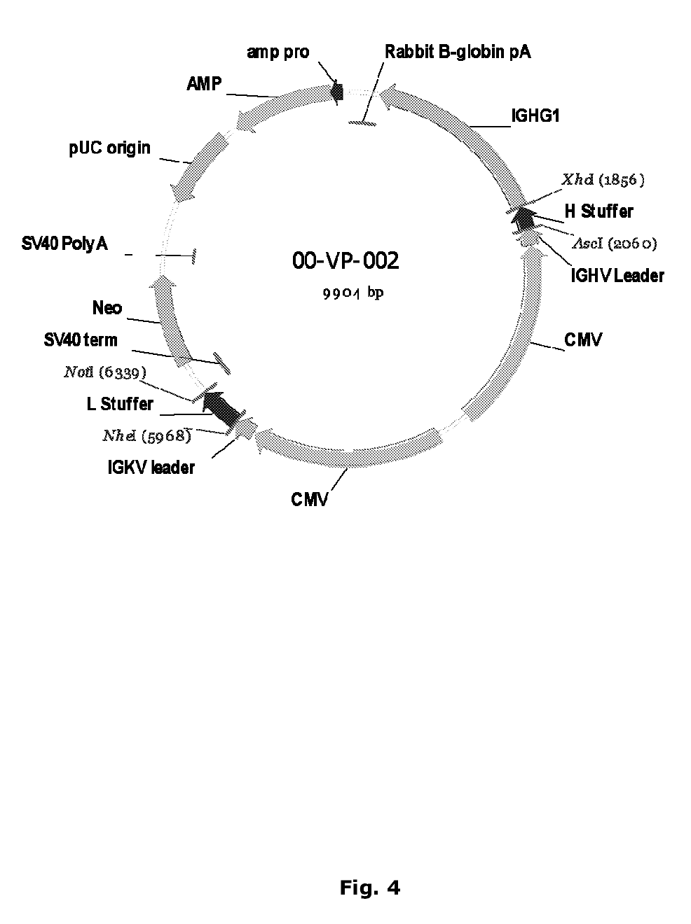

Briefly, the purified PCR product of the repertoires of linked VH and VL coding pairs, spliced to the human kappa constant coding region, were cleaved with XhoI and NotI DNA endonucleases at the recognition sites introduced into the termini of PCR products. The cleaved and purified fragments were ligated into an XhoI/NotI digested mammalian IgG expression vector, OO-VP-002 (FIG. 4) by standard ligation procedures. The ligation mix was electroporated into E. coli and added to 2×YT plates containing the appropriated antibiotic and incubated at 37° C. over night. The amplified repertoire of vectors was purified from cells recovered from the plates using standard DNA purification methods (Qiagen). The plasmids were prepared for insertion of promoter-leader fragments by cleavage using AscI and NheI endonucleases. The restriction sites for these enzymes were located between the VH and VL coding gene pairs. Following purification of the vector, an AscI-NheI digested bi-directional mammalian promoter-leader fragment was inserted into the AscI and NheI restriction sites by standard ligation procedures. The ligated vector was amplified in E. coli and the plasmid was purified using standard methods. The generated repertoire of screening vectors was transformed into E. coli by conventional procedures. Colonies obtained were consolidated into 384-well master plates and stored. The number of arrayed colonies exceeded the number of input PCR products by at least 3-fold, thus giving 95% percent likelihood for presence of all unique V-gene pairs obtained. Screening for binding to CD5 extracellular domain

In general, the screening was made as a two step procedure. The antibody-libraries were screened for reactivity to recombinant CD5-ECD protein in ELISA after which Flow Cytometry was used as a cell based approach, with the CD5-transfected DG05.2 cell line, for detection of anti-CD5 antibodies binding to cell-surface expressed CD5. Briefly for the ELISA, Nunc maxisorb plates (cat no 464718) were coated with 20 μl of 5 μg/ml CD5-ECD protein (CD5-ECD was isolated by TEV-protease cleavage and subsequent purification on a Nickel column), diluted in PBS at 4° C. over night. The next day the wells were blocked in 50 ul 1%-BSA-PBS-T for 1 hour at RT and subsequently were washed four times with PBS+0.05% tween 20 (PBS-T) before 13 μl of 1%-BSA-PBS-T and 2 μl supernatants from CHO-flp-019 transfectants (see below) were added and incubated for 1½ hour R.T. Then the plates were washed once with PBS-T 20 μl per well secondary antibody (HRP-Goat-anti-human IgG, Jackson, cat no 109-035-097) diluted 1:5000 in 1% BSA-PBS-T was added to detect the antibodies present in the supernatant and incubated for 1 hour at Room Temperature. The plates were washed four times in PBS-T before addition of 25 μl substrate (Kem-en-tec Diagnostics, cat no 4390) that was incubated for 5 min. 25 μl 1M sulfuric acid was added after the incubation to stop the reaction. Specific signal was detected on an ELISA reader at 450 nm.

For the cell based Flow cytometry detection of anti-CD5 antibodies, DG05.2 cells transfected with CD5-full length were used. Cells were cultured in MEM-alpha medium supplemented with 10% FBS (Fetal Bovine Serum) and 1% Penicillin Streptomycin. Before use for screening the cells were washed in PBS, trypsinized with TrypLE and resuspended in growth medium. Subsequently the cell suspensions were washed twice in PBS by centrifugation at 250×g for 5 min, dislodging and resuspended in 5 ml 1% FBS-PBS. The cells were counted and diluted to 500000 cells/ml, 25 μl of this solution was mixed with 25 μl of 40 μg/ml anti-CD5 antibody diluted in 1% FBS-PBS and incubated 30 mins at 4° C. in the dark. The cell-antibody suspensions were washed twice in PBS by centrifugation at 250×g for 5 min, dislodged and 50p1 of APC-conjugated mouse anti-human IgG antibody (BD Pharmingen cat. No. 550931) was added before incubation 30 mins at 4° C. in the dark. The cell-antibody suspensions were washed twice in PBS by centrifugation at 250×g for 5 min, dislodged, resuspended in 100 μl 1%-BSA-PBS and analysed by use of a FACS Calibur equipped with an HTS unit.

The data from the screening indicates that 68 (11.7%) of the total clones were positive in the ELISA. 37 unique clonotypes were identified. 15 of the 37 clonotypes were also positive in FACS. All the unique clonotypes were selected for further analysis.

Sequence Analysis and Clone Selection

The clones identified as CD5-specific in ELISA were retrieved from the original master plates (384-well format) and consolidated into new plates. DNA was isolated from the clones and submitted for DNA sequencing of the V-genes. The sequences were aligned and all the unique clones were selected. Multiple alignments of obtained sequences revealed the uniqueness of each particular clone and allowed for identification of unique antibodies. Following sequence analysis of 68 clones, 37 genetically distinct antibody sequence clusters were identified. These clusters of related sequences have probably been derived through somatic hypermutations of a common precursor clone. Overall, one clones from each cluster was chosen for validation of sequence and specificity. Sequences of selected antibody variable sequences are shown in Appendix 1.

Sequence and Specificity Validation

In order to validate the antibody encoding clones, DNA plasmid was prepared and transfection of FreeStyle CHO-S cells (Invitrogen) in 2-ml scale was performed for expression. The supernatant were harvested 96 hours after transfection. The specificity was determined by CD5-specific ELISA.

TABLE 9
Immunization schedules used to generate starting material for anti-CD5
antibody cloning.

| Schedule, | | | | | | |
|---|---|---|---|---|---|---|
| Mouse | | Injec- | Injec- | Injec- | Injec- | |
| group | Strain | tion 1 | tion 2 | tion 3 | tion 4 | Termination |
| 9 | Balb/c | Day 1 | Day 28 | Day 49 | Day 70 | Day 76 |
| | | 50 μg | 50 μg | 50 μg | 50 μg | |
| | | CD5- | CD5- | CD5- | CD5- | |
| | | hGH | hGH | hGH | hGH | |
| | | 50 μl | 50 μl | 50 μl | 50 μl | |
| | | TT + | TT + | TT + | TT + | |
| | | 50 μl | 50 μl | 50 μl | 50 μl | |
| | | CFA | IFA | IFA | IFA | |
| | | s.c. | s.c | s.c | s.c | |

TABLE 10
RT-PCR multiplex overlap-extension primer mix.

| Primer | Conc. | | SEQ |
|---|---|---|---|
| Name | (nM) | Sequence | ID |
| mHCrev1 | 0.2 | GACSGATGGGCCCTTGGTGG | 1 |
| mKappar1 | 0.2 | GCTGTAGGTGCTGTCTTTGC | 2 |
| mVH set | | | |
| mVH A | 0.04 | TATTCCCATGGCGCGCCSAGGTCCARCTGCARCAGYCTG | 3 |
| mVH B | 0.04 | TATTCCCATGGCGCGCCGARGTGMAGCTKGTKGAGTC | 4 |
| mVH C | 0.04 | TATTCCCATGGCGCGCCSAGGTGCAGCTKMAGGAGTC | 5 |
| mVH 8 | 0.04 | TATTCCCATGGCGCGCCCAGGTTACTCTGAAAGAGTC | 6 |
| mVH 9 | 0.04 | TATTCCCATGGCGCGCCCAGATCCAGTTGGTGCAGTCTG | 7 |
| mVK set | | | |
| mVK D | 0.04 | GGCGCGCCATGGGAATAGCTAGCCGAYATCCAGATGACHCARWCT | 8 |
| mVK E | 0.04 | GGCGCGCCATGGGAATAGCTAGCCRACATTGTGMTGACHCAGTC | 9 |
| mVK F | 0.04 | GGCGCGCCATGGGAATAGCTAGCCSAMATTGTKCTSACCCARTCTC | 10 |
| mVK 1-2 | 0.04 | GGCGCGCCATGGGAATAGCTAGCCGATRTTGTGATGACBCARRCT | 11 |

W = A/T, R = A/G, S = G/C, Y = C/T, K = G/T, M = A/C, H = ACT, B = GCT; Conc.-final concentration.

TABLE 11
Nested primer set.

| Primer | Conc. | | SEQ |
|---|---|---|---|
| name | (nM) | Sequence | ID |
| mHCrev1- | 0.2 | GGACAGGGMTCCAKAGTTCCADKT | 12 |
| ext | | | |
| hmJK set | | | |
| hmJK1-v2 | 0.2 | GACAGATGGTGCAGCCACAGTTCGTTTGATTTCCAGCTTGGTG | 13 |
| hmJK2-v2 | 0.2 | GACAGATGGTGCAGCCACAGTTCGTTTTATTTCCAGCTTGGTC | 14 |
| hmJK4-v2 | 0.2 | GACAGATGGTGCAGCCACAGTTCGTTTTATTTCCAACTTTGTC | 15 |
| hmJK5-v2 | 0.2 | GACAGATGGTGCAGCCACAGTTCGTTTCAGCTCCAGCTTGGTC | 16 |

K = G/T, M = A/C, D = AGT; Conc.-final concentration.

TABLE 12
Kappa constant splicing primer set.

| | | | |
|---|---|---|---|
| | Conc. | | SEQ |
| Primer | (nM) | Sequence | ID |

Human kappa constant amplification

| | | | |
|---|---|---|---|
| hKCforw- | 0.2 | GAACTGTGGCTGCACCATCTGTC | 17 |
| v2 | | | |
| Kappa3′ | 0.2 | ACCGCCTCCACCGGCGGCCGCTTATTAACACTCTCCCCTGTTG | 18 |

Splicing by overlap extension

| | | | |
|---|---|---|---|
| mhKCrev | 0.2 | ACCGCCTCCACCGGCGGCCGCTTATTAACACTCTCCCCTGTTGAAGCTCTT | 19 |

mJH set

| | | | |
|---|---|---|---|
| mJH1 | 0.2 | GGAGGCGCTCGAGACGGTGACCGTGGTCCC | 20 |
| mJH2 | 0.2 | GGAGGCGCTCGAGACTGTGAGAGTGGTGCC | 21 |
| mJH3 | 0.2 | GGAGGCGCTCGAGACAGTGACCAGAGTCCC | 22 |
| mJH4 | 0.2 | GGAGGCGCTCGAGACGGTGACTGAGGTTCC | 23 |

### Example 2

### Mammalian Production of Anti-CD5 Antibodies

The Freestyle MAX CHO expression system (Invitrogen) was used for transient expression of anti-EGFR antibodies. Antibodies were expressed in 200 -2000 ml volume.

Approximately 24 hours before transfection CHO-S cells were passaged to reach a cell concentration of 0.5×106 cells/ml. Plasmid (1.25 μg per ml cell culture media) was diluted into OptiPro serum-free medium and mixed with a solution of FreeStyle MAX Transfection reagent as recommended by the supplier. The transfection reagents were transferred to the cell culture and supernatant were harvested 8 days later.

The expressed antibodies were purified from the culture supernatant using an affinity chromatography step employing a Protein A-Sepharose column (MabSelect Sure, GE Health Care) for purification of IgG1 molecules. The antibodies were eluted from the column using 0.1 M Glycine, 2.7. The fractions containing antibodies, determined by absorbance measurements at 280 nm, were pooled and dialyzed against 1× PBS.

### Example 3

### Determination of Epitope Specificities

Competition ELISA with Reference Antibodies

By using reference antibodies binding to CD5, a competition ELISA was developed that could distinguish between the binding epitopes of anti-CD5 antibodies by incubation with a secondary reagent that was specific for the human Fc region of anti-CD5 antibodies and exhibiting no cross reactivity to murine IgG Fc. The ELISA was adapted from the descriptions published in Ditzel et al, 1995, The Journal of Immunology, Vol 154, Issue 2 893-906.

An epitope blocking ELISA was performed by diluting CD5-ECD antigen to 1 μg/ml in PBS; and coating 50 μl/ELISA well overnight at 4° C. The next morning wells were washed twice with PBS-T and blocked for one hour with PBS-T-1% BSA at room temperature followed by wash four times in PBS-T. Next 25 μl murine reference mAbs were added to independent ELISA wells in a dilution known from previous experiments to saturate all epitopes on CD5 in this concentration. After 15 min, 25 μl supernatant containing anti-CD5 antibodies was to wells preincubated with reference antibodies or wells containing 25 μl PBS. Antibodies were incubated for 45 min. at room temperature after which wells were washed four times with PBS-T. A secondary Goat-anti-Human IgG HRP conjugate was diluted 1:3000 and 50 μl was added to each well followed by 30 min incubation at room temperature. Finally wells were washed four times with PBS-T and plates were developed by adding 50 μl/well TMB and read at 620 nm every 5-15-30 min before the reaction was stopped with 50 μl/well 1 M H2SO4 and read at 450 nm. The degree of inhibition was calculated from the formula: % inhibition=(1−(OD competition/OD no competition (PBS)))×100.

ELISA Reagents:
1) Coating buffer: 1× PBS; Gibco cat: 20012-019
2) Antigens: CD5-ECD
3) ELISA plate: NUNC Maxisorp; cat: 442404
4) Blocking/Dilution buffer: 1% BSA in PBS-T (PBS-T-1% BSA)
5) Washing buffer: 1× PBS/0.05% Tween 20 (PBS-T)
6) Reference antibodies:
   UCHT-2 (murine), BD Pharmingen, 555350
   BL1a (murine), Beckman Coulter, IM116
   1804 (murine), AbD Serotech, MCA1804
   L17F12 (murine), BD Pharmingen, 3463000
   H65 (murine), Abcam, ab20132
   MEM-32 (murine), Abcam, ab9189
   CRIS-1 (murine), Abcam, ab36466
7) Goat-anti-Human IgG HRP conjugate; Serotec, Star 106P
8) TMB Plus; KemEnTec, cat #4390L
9) 1 M H₂SO₄

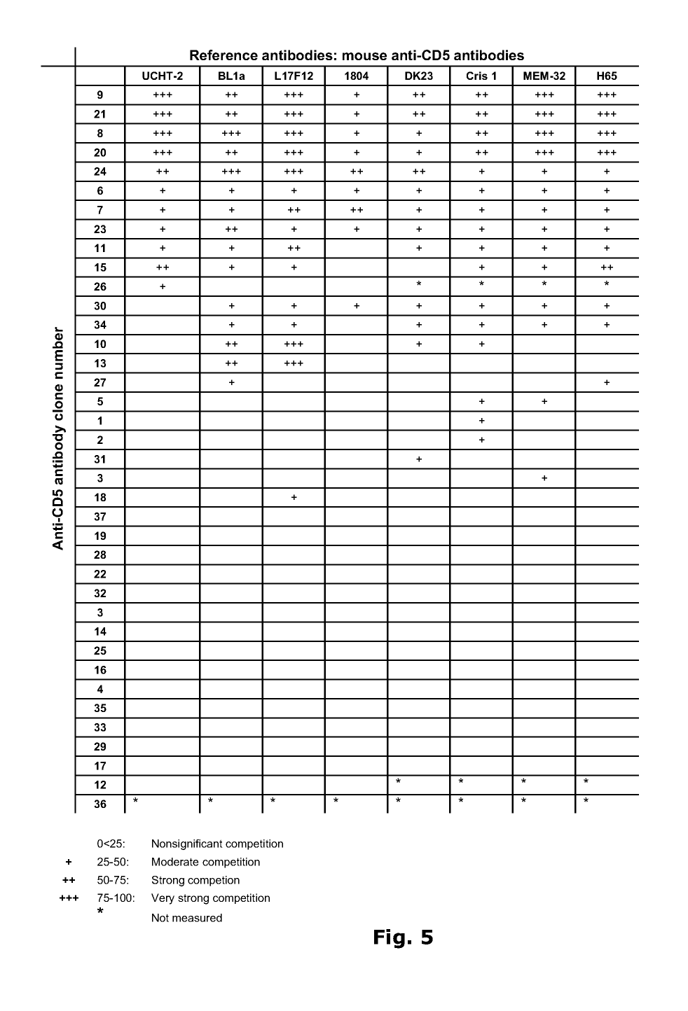

ELISA competition assays were employed to rank Anti-CD5 antibody supernatants according to the specificity of used reference antibodies raised against the CD5 ECD. Inhibition values from 50-100% were taken as an indication of significant competition between antibody pairs binding overlapping epitopes or epitopes in close proximity on the antigen, while inhibition values below 50% indicated that the recognized epitopes by the antibody pairs were not in close proximity resulting in decreased steric hindrance. The Anti-CD5 antibodies were found to bind a variety of epitopes on CD5 (FIG. 5). For some antibodies we observed no competition with the reference antibodies, as the reference antibodies presumably bind epitopes on Domain 1, these antibodies most likely binds Domain II or Domain III.

Competition Analysis for Distinct Epitopes with Reference or same Species Antibodies Using Surface Plasmon Resonance Technology

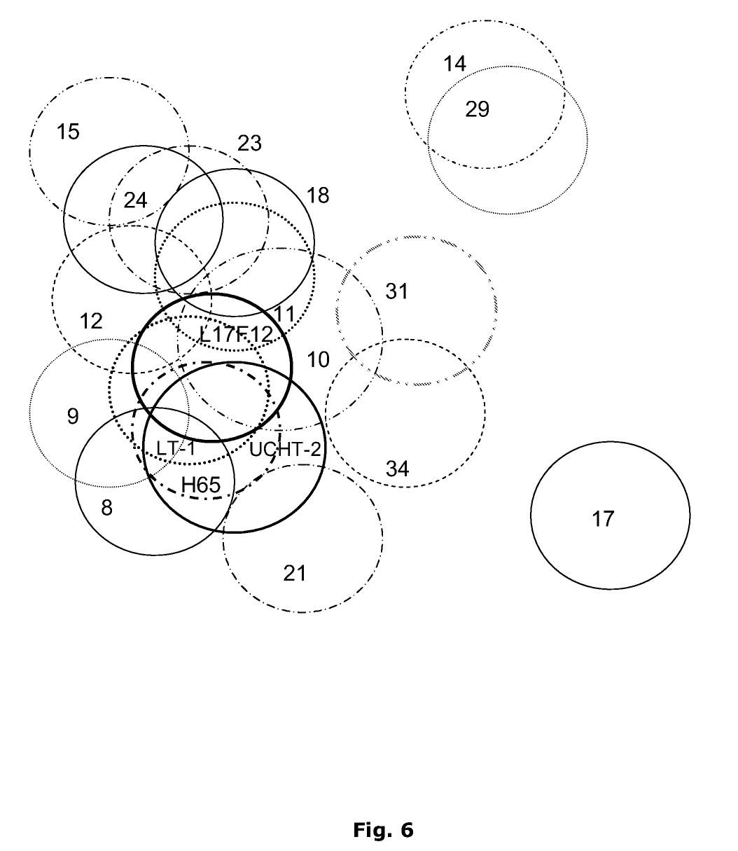

SPR analysis was performed on a Biacore 2000 machine containing four flow cells. A CM5 Biacore chip was conjugated with 8.000 Resonance units (Ru) polyclonal anti-human IgG Fc-specific antibody to flow cells 1-4 according to the manufacturer's instructions. Using a flow rate of 5 μl/min, 5 μl Erbitux and 5 μl of one anti-CD5 antibody clone at a concentration of 40 μg/ml, was injected and captured in flow cell 1 and flow cells 2-4, respectively, to which anti-human IgG Fc-specific antibody had been conjugated. Subsequently, 2 times 15 μl Synagis at a concentration of 10 mg/ml was injected to block the remaining sites of the anti-human IgG Fc-specific antibodies. After overload of Synagis was washed out, 15 μl of 200 nM CD5-ECD was injected over all four flow cells at a flow rate of 5 μl/min and captured by the anti-CD5 antibody clone in flow cell 2-4. This was followed by injection of different anti-CD5 antibody clones, which bind CD5 if the anti-CD5 antibody clone capturing CD5 did not bind overlapping epitopes, FIG. 6. The antibody/antigen complex was then stripped with a low pH acid wash (30 sec. contact time with 100 mM H3PO4) and the whole cycle was then repeated until all the Anti-CD5 antibody clones were tested for binding to CD5 simultaneously. The binding of the second antibody clone binding to CD5 after this has been captured by the first antibody clone, was calculated as follows: First; the reference sensogram in flow cell 1 was withdrawn from the sensograms in flow cell 2-4. Second; the amount of bound second antibody per bound CD5 was calculated: (RU before second Anti-CD5 antibody binding/RU after second Anti-CD5 antibody binding)/(RU before CD5 binding/RU after CD5 binding).

Reagents:
1. CM5 chip; Biacore, Cat. No. BR-1000-14
2. NHS; Biacore BR-1000-50
3. EDC; Biacore BR-1000-50
4. 10 mM Acetate buffer pH 4.5; Biacore, Cat. No. BR-1003-50
5. Goat anti-human IgG Fc antibody; Caltag, Cat. No. H10500
6. Ethanolamine, 1.0M pH 8.5; Biacore BR-1000-50
7. 10× HBS-EP running buffer: 0.01 M HEPES pH 7.4, 0.15 M NaCl, 3 mM EDTA, 0.005% v/v Surfactant P20
8. Antigen: Inhouse produced recombinant human CD5 extracellular domain
9. 100 mM H₃PO₄
10. Reference antibodies:
   UCHT-2 (murine), BD Pharmingen, 555350
   L17F12 (murine), BD Pharmingen, 346300
   H65 (murine), Abcam, ab20132
   LT-1 (murine), Abcam, ab19717
11. Non-CD5 specific control: Erbitux (Merck KGaA, 64271 Darmstadt, Germany, Catalogue #: 018964

Affinity of Anti-CD5 Antibodies Using Surface Plasmon Resonance Technology.

SPR analysis was performed on a Biacore 2000 machine containing four flow cells. A CM5 Biacore chip was conjugated with 8.000 Resonance units (Ru) polyclonal anti-human IgG Fc-specific antibody to flow cells 1-4 according to the manufacturer's instructions. Each anti-CD5 antibody was determined for binding to four different concentrations of CD5-ECD (concentrations for the four cycles are shown below) before the affinity was calculated. The non-CD5 binding antibody, Erbitux served as a negative control and was subtracted from the values obtained with the anti-CD5 specific antibodies. Using a flow rate of 25 μl/min, 25 μl Erbitux and 25 μl of three anti-CD5 antibody clones (all in the same concentration), were injected and captured in flow cell 1, 2, 3 and 4, respectively, to which anti-human IgG Fc-specific antibody had been conjugated. Subsequently, all flow cells were washed and after waiting 500 s, 250 μl of CD5-ECD 100 nM or 200 nM (Cycle 1) was injected in Flow cells 1-4. After waiting 1000 s, 30 μl H3PO4 was injected using a flow rate of 60 μl/min. The antibody/antigen complex was then stripped with a low pH acid wash (30 sec. contact time with 100 mM H3PO4). Cycle 2 was then executed with 50 nM or 100 nM CD5-ECD, followed by cycle 3 with 25 nM or 50 nM CD5-ECD and finally cycle 4 with 12.5 nM or 25 nM CD5-ECD. Four new cycles were then repeated until all the anti-CD5 antibody clones were tested for binding to CD5 in four different concentrations. The association rate constant (ka) and dissociation constant (kd) were evaluated globally by fitting the four binding curves to predefined 1:1 (Langmuir) association and dissociation models with BIAevaluation 4.1 software (Biacore), Table 13.

Reagents:
1. CM5 chip; Biacore, Cat. No. BR-1000-14
2. NHS; Biacore BR-1000-50
3. EDC; Biacore BR-1000-50
4. 10 mM Acetate buffer pH 4.5; Biacore, Cat. No. BR-1003-50
5. Goat anti-human IgG Fc antibody; Caltag, Cat. No. H10500
6. Ethanolamine, 1.0M pH 8.5; Biacore BR-1000-50
7. 10× HBS-EP running buffer: 0.01 M HEPES pH 7.4, 0.15 M NaCl, 3 mM EDTA, 0.005% v/v Surfactant P20
8. Antigen: Inhouse produced recombinant human CD5 extracellular domain
9. 100 mM H₃PO₄
10. Antibodies: Anti-CD5 antibodies
11. Non-CD5 specific control:

Erbitux (Merck KGaA, 64271 Darmstadt, Germany, Catalogue #: 018964

Simultaneously Binding of Anti-CD5 Antibody Clones to CD5 in Biacore

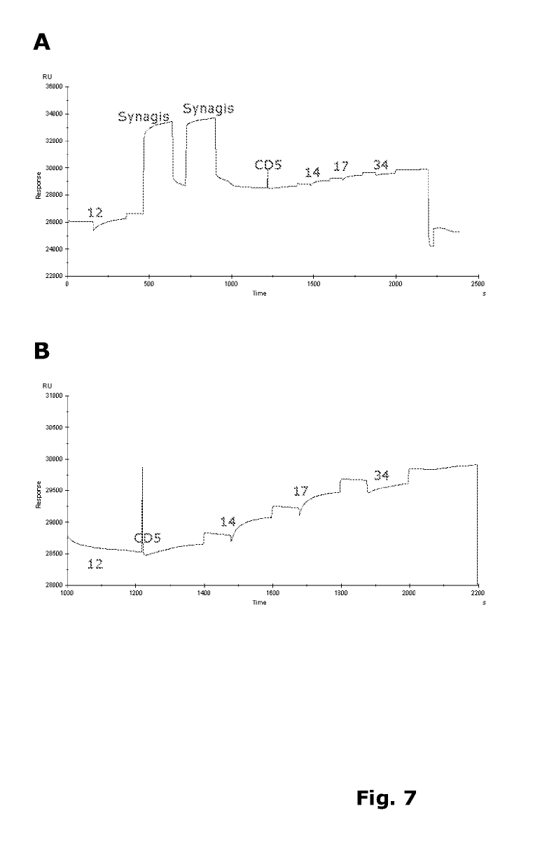

SPR analysis was performed on a Biacore 2000 machine containing four flow cells. A CM5 Biacore chip was conjugated with 8.000 Resonance units (Ru) polyclonal anti-human IgG Fc-specific antibody to flow cells 1-4 according to the manufacturer's instructions. Using a flow rate of 5 μl/min, 15 μl Anti-CD5 antibody Clone 12 (Clone 12) was injected and captured in flow cell 1 to which anti-human IgG Fc-specific antibody had been conjugated. Subsequently, 2 times 15 μl Synagis at a concentration of 10 mg/ml was injected to block the remaining sites of the anti-human IgG Fc-specific antibodies. After overload of Synagis was washed out, 15 μl of 200 nM CD5-ECD was injected over flow cell 1 at a flow rate of 5 μl/min and captured by Clone 12 in flow cell 1. This was followed by injection of 15 μl of Clone 14, which bind CD5 captured by Clone 12. After CD5 was saturated with Clone 14, 15 μl of Clone 17 was injected and after saturation of CD5 with Clone 17, 15 μl of Clone 34 was injected, all in flow cell 1 at a flow rate of 5 μl/min, FIG. 7. The antibody/antigen complex was then stripped with a low pH acid wash (30 sec. contact time with 100 mM H3PO4).

Simultaneously Binding of Anti-CD5 Antibody Clones to CD5 on CEM Cells

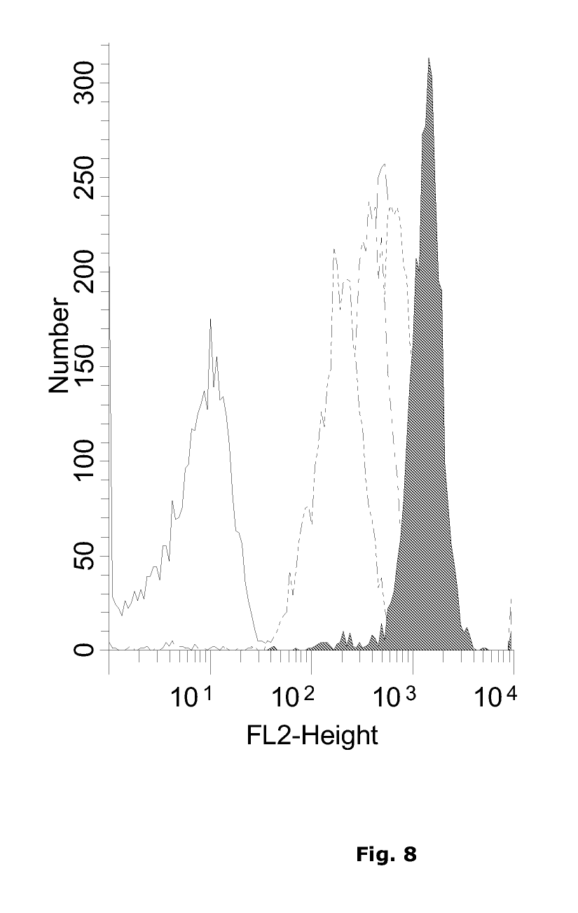

Binding of anti-CD5 antibody clones simultaneously to CD5 on the surface of CEM (ATCC-CCL-119) was performed by Flow Cytometri on a FACS Calibur. 500000 CEM cells were incubated with 50 μl of Anti-CD5 antibody Clone 12, 14, 17, 34 or a mixture of Clone 12, 14, 17 and 34 diluted to 40 μg/ml in 1% FBS-PBS, at 4° C. in the dark for 30 min. Subsequently the cell suspensions were washed twice in PBS by centrifugation at 250×g for 3 min, dislodging and incubated with 20 μl PE-conjugated Goat Anti-human IgG-specific antibody (Beckman Coulter cat. No. IM1626) and 30 μl of 1% FBS-PBS, at 4° C. in the dark for 30 min. Subsequently the cell suspensions were washed twice in PBS by centrifugation at 250×g for 3 min, dislodging and resuspended in 100 μl 1% FBS-PBS before analysis on a FACS-Calibur equipped with an HTS unit, FIG. 8.

### Example 4

### CD5 Internalization

The ability of anti-CD5 antibodies to induce CD5 internalization was investigated by Flow Cytometry. B-CLL cells are purified from patient peripheral blood samples using Ficoll-Hypaque density gradient. Samples from three CLL patients are included in every experiment. Peripheral blood is mixed 1:1 with 1×PBS, 5 ml of this mixture is added on the top of 4 ml Ficoll-Hypaque solution and the tubes are subsequently centrifuged 20 mins at 800×g. The PBMC layer containing the CLL cells is isolated, mixed with 50 ml 1×PBS and centrifuged 5 mins at 1000 rpm. This is repeated twice. The cells are then analysed by Flow cytometry for CD5 and CD19 expression and the percentage of CLL cells in the PBMC population- only samples with higher than 95% CLL cells are used. Cells are subsequently counted, diluted to 5×10⁶ cells/ml in 1×PBS and 150 μl are transferred to each well in a round-bottom 96-well plate. After centrifugation of the plate, 3 mins at 1000 rpm and dispersion of supernatant in each well, the pelleted cells are resuspended in 25 μl PBS. Cells are then incubated with 3.3 μg/ml anti-CD5 antibodies for 18 hours at either 37° C. or 4° C. after which they are washed twice in ice-cold FACS buffer (1×PBS+2% FBS+0.1% Azide) and stained with secondary antibody (FITC-conjugated Goat F(ab′)₂ Anti-human Fc specific IgG, Caltag H10101) diluted 1:20 in ice-cold FACS buffer for 30 min on ice. Incubation below 4° C. completely inhibits internalization. Finally the cells are washed twice and analyzed on a FACS Calibur.

Results

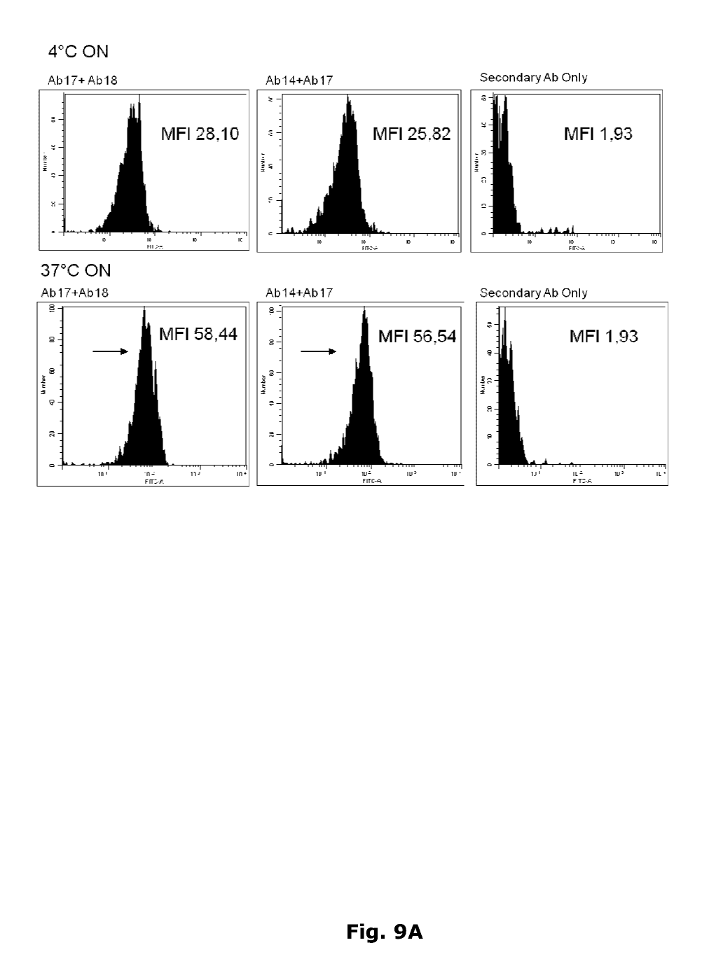

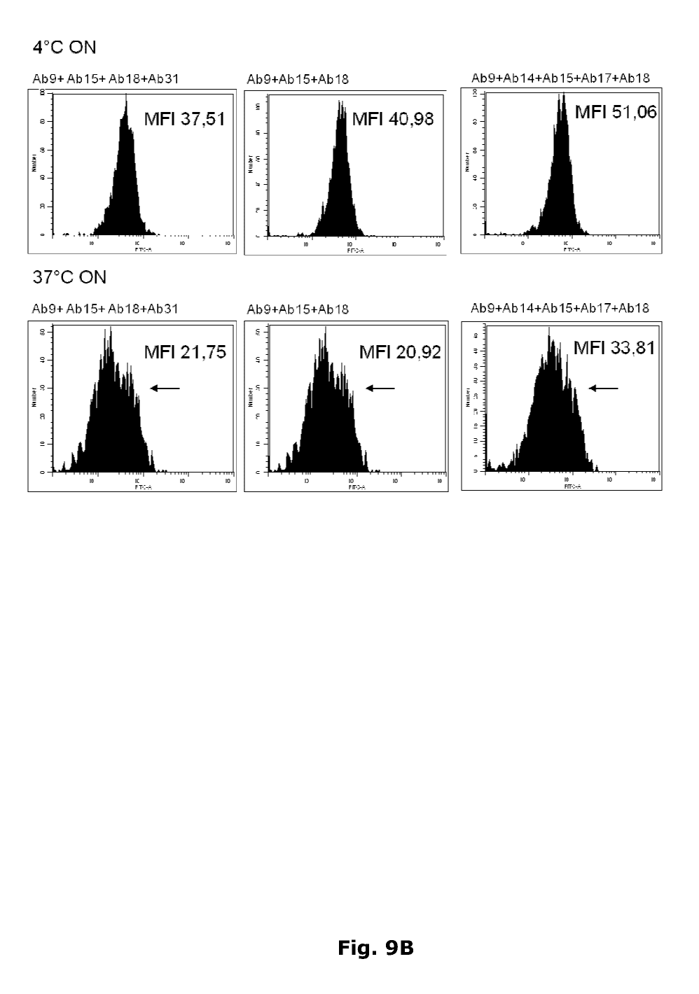

A range of antibody mixtures containing antibodies with non-overlapping epitopes were tested for ability to induce CD5 internalization by flow cytometry (FIG. 9). As is evident from the results presented in FIGS. 9A and 9B some antibody mixtures with non-overlapping epitopes induce internalization (FIG. 9B) whereas others do not (FIG. 9A). As monoclonal antibodies Ab9, Ab14, Ab15, Ab17, Ab18 and Ab31 fail to induce internalization (data not shown). Induction of internalization is thus epitope dependent and it is possible based on knowledge of binding epitope to design antibody mixtures which either yields a high antibody density on the surface of CD5 positive cells or induce CD5 internalization.

### Example 5

### CD5 Degradation

The ability of anti-CD5 antibodies to induce CD5 degradation was investigated by western blot analysis. CLL cells from patients are purified, washed and analyzed as described in Example X and treated with 3.3 μg/ml of anti CD5 antibodies for ½h, 1 h, 2 h, or 4 hours. Cells are then washed again and lyzed in RIPA buffer (50 mM Tris-HCl, 150 mM NaCl, 1 mM PMSF, 1 mM EDTA, 5 μg/ml aprotinin, 5 μg/ml Leupeptin, 1% Triton x-100, 1% sodium deoxycholate and 0.1% SDS). 8 μg of protein is resolved by sodium dodecyl sulfate-polyacrylamide gel electrophoresis and electroblotted onto nitrocellulose membranes. After blocking in 5% non-fat milk, membranes are incubated with primary antibody (mouse anti-CD5 Ab, Clone 4C7 from AbD Serotec) diluted 1:500 overnight at 4° C. followed by washing and incubation with horseradish peroxidase (HRP)-conjugated secondary antibody (Goat anti-mouse IgG, HAF007 from R&D systems) for 1 h at room temperature. The HRP signal is detected using enhanced chemiluminescence plus western blotting detection system (Amersham Biosciences).

Results

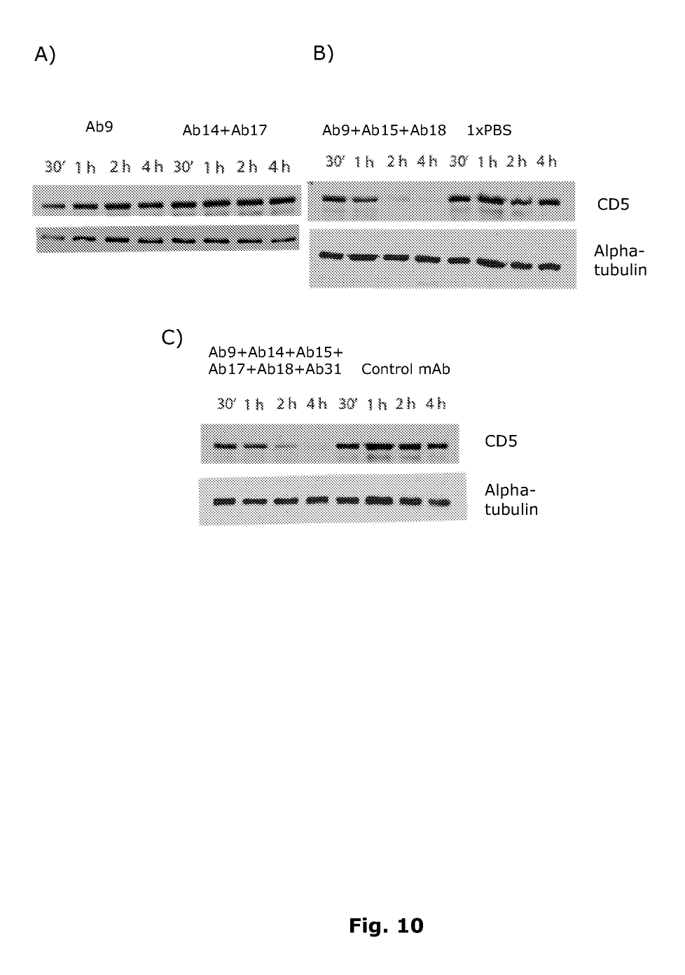

A range of antibody mixtures containing antibodies with non-overlapping epitopes were tested for ability to induce CD5 degradation by western blot analyses (FIG. 10). As is evident from the results presented in FIG. 10 some antibody mixtures with non-overlapping epitopes induce CD5 degradation (FIGS. 10B and C) whereas others do not (FIG. 10A). As monoclonal antibodies Ab9, Ab14, Ab15, Ab17, Ab18 and Ab31 fail to induce CD5 degradation (Only Ab9 is shown in FIG. 10A). Induction of CD5 degradation is thus like CD5 internalization epitope dependent. Neither control mAb nor PBS induces CD5 degradation. CD5 degradation most likely follows CD5 internalization.

1. Antibody composition comprising at least two anti-CD5 antibodies capable of binding distinct CD5 epitopes.

2. Antibody composition according to claim 1, wherein said epitopes are non-overlapping.

3. Antibody composition according to any one of the preceding claims, wherein said antibodies bind at least one extracellular domain of CD5 selected from the group consisting of domain I, domain II, and domain III.

4. Antibody composition according to any one of the preceding claims, wherein at least one antibody binding a distinct CD5 epitope is capable of enhancing the binding of at least one other antibody to a different distinct CD5 epitope.

5. Antibody composition according to any one of the preceding claims, wherein at least one antibody binding a distinct CD5 epitope is capable of increasing the maximum binding capacity of at least one other antibody with respect to CD5.

6. Antibody composition according to any one of the preceding claims, wherein said antibodies are recombinant antibodies.

7. Antibody composition according to any one of the preceding claims, said antibodies being chimeric with murine variable regions and human constant regions.

8. Antibody composition according to claim 7, wherein the human constant region is IgG1 or IgG2.

9. Antibody composition according to any one of the preceding claims, said antibodies being humanised antibodies.

10. Antibody composition according to any one of claims 1-6, said antibodies being human antibodies.

11. Antibody composition according to any one of the preceding claims, wherein at least one of said anti-CD5 antibodies has a K_{d} value of 10⁻⁸M or less.

12. Antibody composition according to any one of the claims 1-10, wherein at least one of said anti-CD5 antibodies has a K_{d} value of 10⁻⁹M or less.

13. Antibody composition according to any one of the claims 1-10, wherein at least one of said anti-CD5 antibodies has a K_{d} value of 10⁻¹⁰M or less.

14. Antibody composition according to any one of the preceding claims, wherein said CD5 is human CD5.

15. Antibody composition according to claim 14, wherein said human CD5 has the sequence of as indicated in Table 7.

16. Antibody composition according to any one of the preceding claims, wherein said antibodies are capable of binding to non-human mammal CD5.

17. Antibody composition according to claim 16, wherein said antibodies are capable of binding said non-human mammal CD5 with an affinity substantially identical to the binding affinity of said antibodies to human CD5.

18. Antibody composition according to claim 16 or 17, wherein said mammal is a primate.

19. Antibody composition according to claim 18, wherein said primate is cynomolgous monkey (Macaca fascicularis).

20. Antibody composition according to any of the preceding claims, wherein at least one antibody molecule comprises a constant domain kappa light chain sequence as indicated in Table 6.

21. Antibody composition according to any of the preceding claims, wherein all antibody molecules of the composition comprises a constant domain kappa light chain sequence as indicated in Table 6.

22. Antibody composition according to any of the preceding claims, wherein at least one antibody molecule comprises a constant domain heavy chain sequence as indicated in Table 6.

23. Antibody composition according to any of the preceding claims, wherein all antibody molecules of the composition comprises a constant domain heavy chain sequence as indicated in Table 6.

24. A bi-specific binding molecule having the binding specificities of the antibody composition according to any one of the preceding claims.

25. The bi-specific binding molecule of claim 24, being a dual-variable-domain antibody.

26. The bi-specific binding molecule of claim 24, being a bi-specific Fab-fragment or a bi-specific scFV.

27. Antibody composition according to any of the preceding claims comprising at least one anti-CD5 antibody molecule selected from the group consisting of antibodies having the CDRs of antibodies: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and 37 as indicated in table 1.

28. Antibody composition according to any of the preceding claims comprising at least two anti-CD5 antibody molecules selected from the group consisting of antibodies having the CDRs of antibodies: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and 37 as indicated in table 1.

29. Antibody composition according to any of the preceding claims comprising at least three anti-CD5 antibody molecules selected from the group consisting of antibodies having the CDRs of antibodies: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and 37 as indicated in table 1.

30. Antibody composition according to any of the preceding claims comprising at least four anti-CD5 antibody molecules selected from the group consisting of antibodies having the CDRs of antibodies: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and 37 as indicated in table 1.

31. Antibody composition according to any of the preceding claims comprising at least five anti-CD5 antibody molecules selected from the group consisting of antibodies having the CDRs of antibodies: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and 37 as indicated in table 1.

32. Antibody composition according to any of the preceding claims comprising at least six anti-CD5 antibody molecules selected from the group consisting of antibodies having the CDRs of antibodies: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and 37 as indicated in table 1.

33. Antibody composition according to any of the preceding claims, wherein all anti-CD5 antibody molecules of said composition are selected from the group consisting of antibodies having the CDRs of antibodies: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and 37 as indicated in table 1.

34. Antibody composition according to any one of the preceding claims, wherein said composition comprises at least one further distinct anti-CD5 antibody molecule, wherein said further antibody molecule binds a third distinct epitope.

35. Antibody composition according to any one of the preceding claims, wherein said composition comprises at least one further distinct anti-CD5 antibody molecule, wherein said further antibody molecule binds a fourth distinct epitope.

36. Antibody composition according to any one of the preceding claims, wherein said composition comprises at least one further distinct anti-CD5 antibody molecule, wherein said further antibody molecule binds a fifth distinct epitope.

37. Antibody composition according to any one of the preceding claims, wherein said composition comprises at least one further distinct anti-CD5 antibody molecule, wherein said further antibody molecule binds a sixth distinct epitope.

38. Antibody composition according to any one of claims 34-37, wherein at least 3 of the anti-CD5 antibody molecules comprised in said composition do not inhibit the binding to CD5 of each other.

39. Antibody composition according to any one of claims 35-37, wherein at least 4 of the anti-CD5 antibody molecules comprised in said composition do not inhibit the binding to CD5 of each other.

40. Antibody composition according to any one of claims 36-37, wherein at least 5 of the anti-CD5 antibody molecules comprised in said composition do not inhibit the binding to CD5 of each other.

41. Antibody composition according to claim 37, wherein at least 6 of the anti-CD5 antibody molecules comprised in said composition do not inhibit the binding to CD5 of each other.

42. Antibody composition according to claim 1, said composition comprising a combination of two antibodies selected from the group of combinations of two antibodies consisting of: Ab9+Ab18, Ab9+Ab15, Ab9+Ab31, Ab9+Ab14, Ab9+Ab17, Ab18+Ab15, Ab18+Ab31, Ab18+Ab14, Ab18+Ab17, Ab15+Ab31, Ab15+Ab14, Ab15+Ab17, Ab31+Ab14, Ab31+Ab17, and Ab14+Ab17.

43. Antibody composition according to claim 1, said composition comprising a combination of three antibodies selected from the group of combinations of three antibodies consisting of: Ab9+Ab18+Ab15, Ab9+Ab18+Ab31, Ab9+Ab18+Ab14, Ab9+Ab18+Ab17, Ab9+Ab15+Ab31, Ab9+Ab15+Ab14, Ab9+Ab15+Ab17, Ab9+Ab31+Ab14, Ab9+Ab31+Ab17, Ab9+Ab14+Ab17, Ab18+Ab15+Ab31, Ab18+Ab15+Ab14, Ab18+Ab15+Ab17, Ab18+Ab31+Ab14, Ab18+Ab31+Ab17, Ab18+Ab14+Ab17, Ab15+Ab31+Ab14, Ab15+Ab31+Ab17, Ab15+Ab14+Ab17, and Ab31+Ab14+Ab17.

44. Antibody composition according to claim 1, said composition comprising a combination of four antibodies selected from the group of combinations of four antibodies consisting of: Ab9+Ab18+Ab15+Ab31, Ab9+Ab18+Ab15+Ab14, Ab9+Ab18+Ab15+Ab17, Ab9+Ab18+Ab31+Ab14, Ab9+Ab18+Ab31+Ab17, Ab9+Ab18+Ab14+Ab17, Ab9+Ab15+Ab31+Ab14, Ab9+Ab15+Ab31+Ab17, Ab9+Ab15+Ab14+Ab17, Ab9+Ab31+Ab14+Ab17, Ab18+Ab15+Ab31+Ab14, Ab18+Ab15+Ab31+Ab17, Ab18+Ab15+Ab14+Ab17, Ab18+Ab31+Ab14+Ab17, and Ab15+Ab31+Ab14+Ab17.

45. Antibody composition according to claim 1, said composition comprising a combination of five antibodies selected from the group of combinations of five antibodies consisting of: Ab9+Ab18+Ab15+Ab31+Ab14, Ab9+Ab18+Ab15+Ab31+Ab17, Ab9+Ab18+Ab15+Ab14+Ab17, Ab9+Ab18+Ab31+Ab14+Ab17, Ab9+Ab15+Ab31+Ab14+Ab17, and Ab18+Ab15+Ab31+Ab14+Ab17.

46. Antibody composition according to claim 1, said composition comprising the antibodies: Ab9+Ab18+Ab15+Ab31+Ab14+Ab17.

47. Antibody composition according to claim 1, said composition comprising a combination of antibodies selected from the group of combinations of antibodies consisting of: 9+14+15+17+18+31, 9+14+15+17+18, 9+15+18+31, and 9+15+18;
wherein said composition is capable of causing internalization of CD5.

48. Antibody composition according to claim 1, said composition comprising a combination of antibodies selected from the group of combinations of antibodies consisting of: 14+17, 17+18;
wherein said composition is not capable of causing internalization of CD5.

49. Pharmaceutical composition comprising as an active ingredient an antibody composition according to any one of the claims 1-48 or a bi-specific binding molecule of any of the claims 24-26.

50. An antibody composition according to any one of claims 1-48 or a bi-specific binding molecule of any of the claims 24-26 for use as a medicament.

51. Composition or bi-specific binding molecule according to claim 50 for use in the treatment or prevention of cancer.

52. Composition or bi-specific binding molecule according to claim 50 for use in the treatment or prevention of chronic lymphocytic leukaemia.

53. Composition or bi-specific binding molecule according to claim 50 for use in the treatment or prevention of rheumatoid arthritis.

54. Use of an antibody composition according to any one of claims 1-48 or a bi-specific binding molecule of any of the claims 24-26 in the manufacture of a medicament.

55. A method of treatment comprising administering to a patient in need thereof a pharmaceutical composition according to claim 49.

56. A method for manufacturing an antibody composition comprising:
a. transfecting a first population of eukaryotic cells with a first expression construct coding for a first antibody comprising a first cognate pair of VH and VL chains capable of binding a first distinct CD5 epitope;
b. transfecting a second population of eukaryotic cells with a second expression construct coding for a second antibody comprising a second cognate pair of VH and VL chains capable of binding a second distinct CD5 epitope;
c. optionally repeating step b) for third or further populations, expression constructs, cognate pairs, and CD5 epitopes;
d. selecting transfected first, second and optionally further cell populations;
e. combining the transfected populations in one pot to obtain a cell bank;
f. culturing cells from the cell bank under conditions allowing expression of the antibodies; and
g. recovering and purifying the antibody composition from the supernatant.

57. The method of claim 56, wherein the antibody composition is an antibody composition of any of the claims 1 to 48.

58. The method of claim 56, wherein the cells are transfected with site-specific integration.

59. The method of claim 56, wherein the VH and VL regions are murine and the constant regions of the antibodies are human.

60. The method of claim 59, wherein all antibodies comprise the same heavy chain constant region.

61. A cell bank comprising at least two sub-populations of eukaryotic cells, wherein each sub-population is transfected or transduced with one expression construct coding for an antibody comprising a cognate pair of VH and VL chains capable of binding a distinct CD5 epitope.

62. The cell bank of claim 61, wherein the cell bank encodes an antibody composition of any of the claims of the claims 1 to 48.

63. The cell bank of claim 61, wherein the cells are transfected using site-specific integration.

64. A method of killing cells expressing CD5 comprising administering to cells expressing CD5 an antibody composition of any of the claims 1 to 48 or a bi-specific binding molecule of any of the claims 24-26, and thereby killing the CD5 expressing cells.
